(19) United States
(12) Patent Application Publication
(10) Pub. No.: US 2025/0019425 A1
(43) Pub. Date: Jan. 16, 2025

(54) TRANSTHYRETIN (TTR) MONOMER BINDING ANTIBODIES

(71) Applicant: ADRX, INC., Thousand Oaks, CA (US)

(72) Inventors: David James Leibly, Stevenson Ranch, CA (US); Anna Rose Lay, Los Angeles, CA (US); Marcin Izydor Apostol, Westlake Village, CA (US); Margaret Lee Karow, Santa Rose Valley, CA (US); Tracey Elizabeth Mullen, Quincy, MA (US); Colby Amsden Souders, Weymouth, MA (US)

(21) Appl. No.: 18/708,912

(22) PCT Filed: Nov. 14, 2022
(86) PCT No.: PCT/US22/79810

§ 371 Date: May 9, 2024

Related U.S. Application Data
(60) Provisional application No. 63/278,925, filed on Nov. 12, 2021.

(51) Int. Cl. C07K 16/18; G01N 33/68

(52) U.S. Cl. CPC C07K 16/18; G01N 33/6893; C07K 2317/24; C07K 2317/92; G01N 2470/04

(57) ABSTRACT

The disclosure provides antibodies that bind to forms of human transthyretin (TTR), monomer TTR specific antibodies, and methods of using the same.

### INCORPORATION BY REFERENCE OF THE SEQUENCE LISTING

Incorporated by reference in its entirety is a computer-readable nucleotide/amino acid sequence listing submitted concurrently herewith and identified as follows: 57198A_Seqlisting.txt; Size: 170,871 bytes: Created: Nov. 7, 2022. To the extent there are differences between the computer-readable sequences and sequences in the specification or figures, the specification and/or figures are controlling.

### TECHNICAL FIELD

The disclosure provides antibodies that bind human transthyretin (TTR) monomer and methods of using these antibodies. The disclosure also provides antibodies that sequester human transthyretin (TTR) monomer. The disclosure provides antibodies that inhibit aggregation of human transthyretin (TTR) or TTR folding intermediates.

### BACKGROUND

Transthyretin (TTR) is a soluble homotetrameric plasma protein composed of 127-amino acid, 14 kDa monomeric subunits. TTR is also known as CTS, HsT2651, PALB, prealbumin, and TBPA. TTR is an amyloidogenic protein that can form fibrils and other aggregates.

In its native state, TTR (or “wild-type” TTR) exists as a tetramer. However, TTR can be converted in an acid mediated dissociation into a monomeric form. Such monomers are believed to be key in TTR aggregation/amyloid formation. Specifically, TTR monomers can subsequently adopt misfolded conformations and aggregate into TTR oligomers and amyloid fibrils in a process known as amyloidogenesis. Mounting evidence indicates that the active aggregation of misfolded monomeric TTR is a root cause of TTR amyloid diseases. The disclosure provides TTR antibodies useful in treating TTR amyloid diseases.

### SUMMARY OF THE INVENTION

The disclosure provides antibodies that bind to forms of human transthyretin (TTR), anti-monomer TTR specific antibodies, and methods of using the same.

One embodiment of the disclosure provides isolated antibodies that bind to human TTR wherein the antibody binds to human TTR with an EC50 of less than about 200 nM as determined in a Sandwich ELISA; and/or binds to human TTR with a KD of less than about 20 nM as determined in a biolayer interferometry assay.

The disclosure provides methods of treating TTR aggregation-related diseases with antibodies of the disclosure and medicaments comprising antibodies of the disclosure. The disclosure provides methods of detecting the monomer form of TTR using antibodies of the disclosure. The disclosure provides methods of diagnosing a disease associated with transthyretin (TTR) amyloidosis, or monitoring the treatment of the disease with an anti-TTR therapy.

More specifically, the disclosure provides an isolated antibody that binds to human transthyretin (TTR) wherein the antibody binds to human TTR with an EC50 of less than about 200 nM as determined in a sandwich ELISA; and/or binds to human TTR with a KD of less than about 20 nM as determined in a biolayer interferometry assay. In some aspects, the human TTR is in a monomeric form. In some aspects, the antibody binds monomeric TTR with an EC50 of less than 20 nM as determined in an indirect ELISA. In some aspects, the antibody prevents aggregation of TTR. In some aspects, the antibody binds a folding intermediate of TTR.

In some aspects, the antibody comprises a heavy chain variable domain (VH) comprising
(a) a CDR-H1 comprising the amino acid sequence of any one of SEQ ID NOs: 2-8;
(b) a CDR-H2 comprising the amino acid sequence of any one of SEQ ID NOs: 9-15; and
(c) a CDR-H3 comprising the amino acid sequence of any one of SEQ ID NOs: 16-22; and a light chain variable domain (VL) comprising
(d) a CDR-L1 comprising the amino acid sequence of any one of SEQ ID NOs: 23-29;
(e) a CDR-L2 comprising the amino acid sequence of any one of SEQ ID NOs: 30-36; and
(f) a CDR-L3 comprising the amino acid sequence of any one of SEQ ID NOs: 37-43.

The disclosure provides an antibody that binds to human transthyretin (TTR), the antibody comprising a heavy chain variable domain (VH) comprising
(a) a CDR-H1 comprising the amino acid sequence of any one of SEQ ID NOs: 2-8;
(b) a CDR-H2 comprising the amino acid sequence of any one of SEQ ID NOs: 9-15; and
(c) a CDR-H3 comprising the amino acid sequence of any one of SEQ ID NOs: 16-22; and a light chain variable domain (VL) comprising
(d) a CDR-L1 comprising the amino acid sequence of any one of SEQ ID NOs: 23-29;
(e) a CDR-L2 comprising the amino acid sequence of any one of SEQ ID NOs: 30-36; and
(f) a CDR-L3 comprising the amino acid sequence of any one of SEQ ID NOs: 37-43.

In some aspects, the antibody comprises a heavy chain variable domain (VH) comprising
(a) a CDR-H1 comprising the amino acid sequence of SEQ ID NO: 2;
(b) a CDR-H2 comprising the amino acid sequence of SEQ ID NO: 9; and
(c) a CDR-H3 comprising the amino acid sequence of SEQ ID NO: 16; and a light chain variable domain (VL) comprising
(d) a CDR-L2 comprising the amino acid sequence of SEQ ID NO: 23;
(e) a CDR-L2 comprising the amino acid sequence of SEQ ID NO: 30; and
(f) a CDR-L3 comprising the amino acid sequence of SEQ ID NO: 37.

In some aspects, the antibody comprises a heavy chain variable domain (VH) comprising
(a) a CDR-H1 comprising the amino acid sequence of SEQ ID NO: 3;
(b) a CDR-H2 comprising the amino acid sequence of SEQ ID NO: 10; and
(c) a CDR-H3 comprising the amino acid sequence of SEQ ID NO: 17; and a light chain variable domain (VL) comprising
(d) a CDR-L2 comprising the amino acid sequence of SEQ ID NO: 24;
(e) a CDR-L2 comprising the amino acid sequence of SEQ ID NO: 31; and
(f) a CDR-L3 comprising the amino acid sequence of SEQ ID NO: 38.

In some aspects, the antibody comprises a heavy chain variable domain (VH) comprising
(a) a CDR-H1 comprising the amino acid sequence of SEQ ID NO: 4;
(b) a CDR-H2 comprising the amino acid sequence of SEQ ID NO: 11; and
(c) a CDR-H3 comprising the amino acid sequence of SEQ ID NO: 18; and a light chain variable domain (VL) comprising
(d) a CDR-L2 comprising the amino acid sequence of SEQ ID NO: 25;
(e) a CDR-L2 comprising the amino acid sequence of SEQ ID NO: 32; and
(f) a CDR-L3 comprising the amino acid sequence of SEQ ID NO: 39.

In some aspects, the antibody comprises a heavy chain variable domain (VH) comprising
(a) a CDR-H1 comprising the amino acid sequence of SEQ ID NO: 5;
(b) a CDR-H2 comprising the amino acid sequence of SEQ ID NO: 12; and
(c) a CDR-H3 comprising the amino acid sequence of SEQ ID NO: 19; and a light chain variable domain (VL) comprising
(d) a CDR-L2 comprising the amino acid sequence of SEQ ID NO: 26;
(e) a CDR-L2 comprising the amino acid sequence of SEQ ID NO: 33; and
(f) a CDR-L3 comprising the amino acid sequence of SEQ ID NO: 40.

In some aspects, the antibody comprises a heavy chain variable domain (VH) comprising
(a) a CDR-H1 comprising the amino acid sequence of SEQ ID NO: 6;
(b) a CDR-H2 comprising the amino acid sequence of SEQ ID NO: 13; and
(c) a CDR-H3 comprising the amino acid sequence of SEQ ID NO: 20; and a light chain variable domain (VL) comprising
(d) a CDR-L2 comprising the amino acid sequence of SEQ ID NO: 27;
(e) a CDR-L2 comprising the amino acid sequence of SEQ ID NO: 34; and
(f) a CDR-L3 comprising the amino acid sequence of SEQ ID NO: 41.

In some aspects, the antibody comprises a heavy chain variable domain (VH) comprising
(a) a CDR-H1 comprising the amino acid sequence of SEQ ID NO: 7;
(b) a CDR-H2 comprising the amino acid sequence of SEQ ID NO: 14; and
(c) a CDR-H3 comprising the amino acid sequence of SEQ ID NO: 21; and a light chain variable domain (VL) comprising
(d) a CDR-L2 comprising the amino acid sequence of SEQ ID NO: 28;
(e) a CDR-L2 comprising the amino acid sequence of SEQ ID NO: 35; and
(f) a CDR-L3 comprising the amino acid sequence of SEQ ID NO: 42.

In some aspects, the antibody comprises a heavy chain variable domain (VH) comprising
(a) a CDR-H1 comprising the amino acid sequence of SEQ ID NO: 8;
(b) a CDR-H2 comprising the amino acid sequence of SEQ ID NO: 15;
(c) a CDR-H3 comprising the amino acid sequence of SEQ ID NO: 22; and a light chain variable domain (VL) comprising
(d) a CDR-L2 comprising the amino acid sequence of SEQ ID NO: 29;
(e) a CDR-L2 comprising the amino acid sequence of SEQ ID NO: 36; and
(f) a CDR-L3 comprising the amino acid sequence of SEQ ID NO: 43.

In some aspects, the antibody is a monoclonal antibody.

In some aspects, the antibody is a humanized or chimeric antibody.

In some aspects, the antibody comprises a VH domain, wherein the VH domain comprises a sequence comprising at least 85% or at least 95% sequence identity to the sequence of any one of SEQ ID NOs: 46-49. In some aspects, the VH domain comprises the sequence of any one of SEQ ID NOs: 46-49. In some aspects, the antibody comprises a VH domain, wherein the VH domain comprises a sequence comprising at least 85% or at least 95% sequence identity to the sequence of any one of SEQ ID NOs: 55-60. In some aspects, the VH domain comprises the sequence of any one of SEQ ID NOs: 55-60.

In some aspects, the antibody comprises a VL domain, wherein the VL domain comprises at least 85% or at least 95% sequence identity to the sequence of any one of SEQ ID NOs: 51-53. In some aspects, the VL domain comprises the sequence of any one of SEQ ID NOs: 51-53. In some aspects, the VL domain comprises a sequence comprising at least 85% or at least 95% sequence identity to the sequence of any one of SEQ ID NOs: 62-64. In some aspects, the VL domain comprises the sequence of any one of SEQ ID NOs: 62-64.

In some aspects, the disclosure provides an antibody fragment that binds to a monomeric form or a folding intermediate form of human TTR.

In some aspects, the disclosure provides a TTR antibody that does not bind to a peptide having an amino acid sequence selected from any one of GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69), LSPYSY (SEQ ID NO: 70), SYSTTAVV (SEQ ID NO: 71), YTIAALLSPYSYSTTAVV (SEQ ID NO: 72), LGISPMHEHAE (SEQ ID NO: 73); and RRYTIAAMLSPYS (SEQ ID NO: 74).

In some aspects, the disclosure provides an antibody that binds to a conformational epitope.

In some aspects, an antibody of the disclosure is an IgG4 antibody.

In some aspects, the disclosure provides an antibody that binds human TTR with an EC50 of less than about 150 nM, or less than about 100 nM, or less than about 50 nM, or less than about 20 nM, as determined in a sandwich ELISA. In some aspects, the antibody binds human TTR with an EC50 of less than about 20 nM, or less than about 5 nM, or less than about 2 nM, or less than about 1 nM, as determined using an Indirect ELISA.

In some aspects, the disclosure provides an antibody that specifically binds human TTR and competes for binding to human TTR with an antibody of the disclosure. In some embodiments, the antibody that competes for binding is an antibody that inhibits aggregation of monomeric TTR.

The disclosure provides isolated nucleic acids encoding each of the antibodies described herein. The disclosure provides isolated host cells comprising such isolated nucleic acids. The disclosure also provides isolated host cells that express proteins from such isolated nucleic acids.

The disclosure provides a method of producing an antibody that binds to human TTR comprising culturing the host cell described herein under conditions suitable for the expression of the antibody. In some aspects, such method further comprises recovering the antibody from the host cell or host cell culture. Additionally, the disclosure provides an antibody produced by such methods.

The disclosure provides a composition comprising an antibody of the disclosure. The disclosure also provides a pharmaceutical composition comprising an antibody of the disclosure and a pharmaceutically acceptable carrier. In some aspects, the composition is a pharmaceutical composition. In some aspects, the antibody or composition is used in treating, preventing, or diagnosing a disease associated with associated with TTR aggregation.

The disclosure provides antibodies and compositions for use in treating TTR amyloidosis. In some aspects, the TTR amyloidosis is transthyretin amyloidosis (ATTR) cardiomyopathy (ATTR-CM) or transthyretin amyloidosis (ATTR) polyneuropathy (ATTR-PN). In some aspects, the antibodies or compositions of the disclosure are used in treating peripheral TTR amyloidosis, ocular amyloid angiopathy or cerebral amyloid angiopathy. In some aspects, the antibodies or compositions are used in treating a disease selected from Familial Amyloid Polyneuropathy (AP). Familial Amyloid Cardiomyopathy (FAC). ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis. TTR-related hyperthyroxinemia, TTR-related ligament amyloidosis including carpal tunnel syndrome, rotator cuff tears and lumbar spinal stenosis, and preeclampsia.

The disclosure provides methods of treating a subject having a disease associated with TTR amyloidosis with an effective amount of an antibody or a composition of the disclosure. In some aspects, the disease is TTR amyloidosis. In some aspects, the disease is Familial Amyloid Polyneuropathy (AP), Familial Amyloid Cardiomyopathy (FAC). ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, or preeclampsia.

The disclosure provides a TTR antibody as disclosed herein further comprising a detectable label.

The disclosure provides methods of measuring monomeric transthyretin (TTR) in a sample. In some aspects, the method comprises contacting the sample with any of the TTR antibodies disclosed herein and measuring the amount of antibody bound to TTR in the sample. In some aspects, the measuring comprises contacting the sample with a secondary antibody and measuring a complex formed thereby. In some aspects, the method comprises using TTR monomer antibodies Ab3 and Ab5, with one of said antibodies used as a capture antibody and one of said antibodies used as a detection antibody.

The disclosure further provides methods of diagnosing a disease associated with transthyretin (TTR) amyloidosis, or monitoring the treatment of the disease with an anti-TTR therapy comprising assaying the level of monomeric TTR in a sample of a body fluid from a subject, wherein the presence or an elevated level of monomeric TTR in the sample of the subject compared to a control indicates the presence of a disease associated with TTR amyloidosis. In some aspects, the monomeric TTR binds to an antibody of the disclosure.

The disclosure further provides a method comprising contacting a sample from a subject with a sample of a bodily fluid from a subject having or suspected of having transthyretin (TTR) amyloidosis; and detecting or measuring monomeric TTR in the sample. In some aspects, the contacting step uses a contacting antibody selected from TTR monomer antibodies Ab3 and Ab5. In some aspects, the detecting or measuring step comprises contacting a complex formed by the contacting antibody and the TTR with a detecting antibody selected from TTR monomer antibodies Ab3 and Ab5, wherein the contacting antibody is different from the detecting antibody. In some aspects, the method further comprises initiating anti-TTR therapy based on detecting or measuring the monomeric TTR in the sample. In some aspects, the method further comprises adjusting the dose of an anti-TTR therapy based on the measurement of the monomeric TTR in the sample. In some aspects, the anti-TTR therapy comprises administering to the subject an antibody of the disclosure or a composition of the disclosure. In some aspects the bodily fluid is blood.

The disclosure also provides a composition comprising an antibody described herein, wherein the composition (i) is a pharmaceutical composition and further comprises a pharmaceutically acceptable carrier, or (ii) a diagnostic composition. In some aspects, the diagnostic composition further comprises reagents conventionally used in immuno-based or antibody-based diagnostic methods. In some aspects, the diagnostic method measures a TTR monomeric form. In some aspects, the diagnostic method uses the Quanterix platform. In some aspects, the diagnostic method is a Western gel.

In some aspects, an antibody of the disclosure recognizes a conformational epitope on monomeric TTR prepared from F87M L110M TTR that is not present on tetrameric TTR and physiological, cellular full-length TTR. In some aspects, an antibody of the disclosure binds to monomeric forms of TTR mutants. In some aspects, therefore, the antibody recognizes a TTR mutation, wherein the mutation is V30M, D38A and/or V1221. In some aspects, the antibody recognizes a TTR mutation, wherein the mutation is D38A or V1221. Thus, in some aspects, the antibody recognizes TTR comprising the amino acid sequence set forth in any of SEQ ID NOs: 145 (V30M), 146 (D38A), and 147 (V1221). In some aspects, the antibody recognizes TTR comprising the amino acid sequence set forth in SEQ ID NO: 146 and/or 147. In some aspects, the antibody recognizes a TTR mutation, wherein the mutation is F87M or L110M. Thus, in some aspects, the antibody recognizes TTR comprising the amino acid sequence set forth in SEQ ID NO: 143 (F87M) or 144 (L110M). In some aspects, an antibody of the disclosure does not bind to SEQ ID NO: 148 (S112I) because the S112I TTR mutant presents in the form of a dimer. However, it binds to TTR F87M L110M S112I (SEQ ID NO: 152), which comprises the S112I mutation because the triple mutant is present in a stabilized monomeric form. In some aspects, the antibody recognizes triple mutations (or a tri-mutation) in TTR, wherein the mutation is TTR V30M F87M L110M (SEQ ID NO: 149). TTR D38A, F87M, L110M (SEQ ID NO: 150), TTR F87M L110M V1221 (SEQ ID NO: 151), or TTR F87M L110M S112I (SEQ ID NO: 152).

The disclosure provides an antibody, wherein the VH domain comprises at least 85% or at least 95% sequence identity with any of the sequences set forth in FIGS. 6A-6B, 7, 14, and 18-21 (SEQ ID NOs: 81-142).

The disclosure provides an antibody, wherein the VL domain comprises at least 85% or at least 95% sequence identity with any of the sequences set forth in FIGS. 6A-6B, 7, 14, and 18-21 (SEQ ID NOs: 81-142).

The disclosure provides an antibody, wherein the antibody specifically binds to TTR comprising the amino acid sequence set forth in any one of SEQ ID NOs: 80 and 143-147 and 149-152. In some aspects, the antibody specifically binds to TTR comprising the amino acid sequence set forth in SEQ ID NO: 80. In some aspects, the antibody specifically binds to TTR comprising the amino acid sequence set forth in SEQ ID NO: 80. In some aspects, the antibody specifically binds to TTR comprising the amino acid sequence set forth in SEQ ID NO: 143. In some aspects, the antibody specifically binds to TTR comprising the amino acid sequence set forth in SEQ ID NO: 144. In some aspects, the antibody specifically binds to TTR comprising the amino acid sequence set forth in SEQ ID NO: 145 or SEQ ID NO: 149. In some aspects, the antibody specifically binds to TTR comprising the amino acid sequence set forth in SEQ ID NO: 146 or SEQ ID NO: 150. In some aspects, the antibody specifically binds to TTR comprising the amino acid sequence set forth in SEQ ID NO: 147 or SEQ ID NO: 151. In some aspects, the antibody specifically binds to TTR comprising the amino acid sequence set forth in SEQ ID NO: 152.

The disclosure includes a method of producing a cell that produces an antibody that specifically binds to a monomeric form of TTR, the method comprising: immunizing a non-human animal with a monomeric form of TTR; screening blood or tissue from the animal for an antibody which specifically binds to the monomeric form of human TTR but does not bind to wild-type tetrameric TTR or to a dimeric or tetrameric form of TTR; and identifying and isolating a cell from the animal that produces the antibody that specifically binds to the monomeric form of human TTR but does not bind to wild-type tetrameric TTR or to a dimeric or tetrameric form of TTR. In some aspects, the antibody-producing cell is recovered by removal of spleen tissue, lymph nodes or bone marrow of the animal. In some aspects, the antibody-producing cell is a B cell, a T cell, or a stem cell. In some aspects, the animal is a mouse, a rat, a guinea pig, or a rabbit. In some aspects, the method further comprises immortalizing the isolated cell. In some aspects, the method comprises producing a hybridoma cell by somatic fusion of the cell to a myeloma cell. In some aspects, the method comprises culturing the antibody-producing cell. In some aspects, the antibody specifically binds to TTR comprising the amino acid sequence set forth in any one of SEQ ID NOs: 80, 143, and 144. In some aspects of the methods of the disclosure, the antibody does not bind to a peptide having an amino acid sequence consisting of a sequence selected from any one of GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69). LSPYSY (SEQ ID NO: 70). SYSTTAVV (SEQ ID NO: 71), YTIAALLSPYSYSTTAVV (SEQ ID NO: 72), LGISPMHEHAE (SEQ ID NO: 73) and RRYTIAAMLSPYS (SEQ ID NO: 74). In some aspects, the monomeric form of human TTR used to immunize the animal is any one of TTR F87M L110M comprising the amino acid sequence set forth in SEQ ID NO: 80; TTR F87M comprising the amino acid sequence set forth in SEQ ID NO: 143; an TTR L110M comprising the amino acid sequence set forth in SEQ ID NO: 144. The disclosure also provides an antibody produced by such method.

The disclosure provides a method for producing a monoclonal antibody that specifically binds to a monomeric form of human TTR, said method comprising: introducing into a non-human animal at least one of any one of TTR F87M L110M comprising the amino acid sequence set forth in SEQ ID NO: 80; TTR F87M comprising the amino acid sequence set forth in SEQ ID NO: 143; and TTR L110M comprising the amino acid sequence set forth in SEQ ID NO: 144; recovering antibody-producing cells from the animal and rendering these cells into a single cell suspension; generating an immortalized cell line from the single cell suspension; screening the supernatant of the immortalized cell line for the presence of an antibody having binding specifically for a monomeric form of human TTR; and selecting, as the monoclonal antibody, an antibody that specifically binds to the monomeric form of human TTR. In some aspects, said animal is a mouse, a rat, a guinea pig or a rabbit. In some aspects, the antibody-producing cells are B cells. T cells or stem cells. In some aspects, the antibody-producing cells are recovered by removal of spleen tissue, lymph nodes or bone marrow of the animal. In some aspects, the immortalized cell line is a hybridoma cell line produced by somatic fusion of the cells in the single cell suspension to myeloma cells. The disclosure also provides an antibody produced by such method.

The disclosure also provides a method comprising: assaying a library of molecules for a molecule peptide or polypeptide that: (i) binds one or more monomeric TTR peptides having amino acid an amino acid sequence selected from SEQ ID NOs: 80 and 143-147 and 149-152; and (ii) exhibits substantially no binding affinity for a tetrameric human TTR protein having the amino acid sequence of SEQ ID NO: 153, identifying a molecule that exhibits the binding properties of (i) and (ii); and isolating the molecule identified in (b), or isolating a cell that expresses said molecules. In some aspects, the method further comprises assaying to identify a molecule that exhibits the binding properties of (i) and (ii) and that (iii) fails to bind to a peptide having an amino acid sequence consisting of SEQ ID NO: 65, 66, 67, 68, 69, 70, 71, 72, 73, or 74. In some aspects, the molecules comprise antibodies or antigen-binding fragments of antibodies. In some aspects, the isolating step comprises isolating a cell that produces an antibody that exhibits the binding properties of (i) and (ii) and optionally (iii). In some aspects, the method comprises a step, prior to the assaying step, of immunizing a non-human mammal with the one or more monomeric TTR peptides, and the assaying comprises screening antibody molecules produced by the mammal. In some aspects, such method further comprises determining amino acid sequence of at least the complementarity-determining regions (CDR) of the antibody; generating humanized antibody sequences that includes said amino acid sequences of said CDRs; and synthesizing nucleic acid that comprises nucleotide sequences that encode the humanized antibody. In some aspects, such method further comprises transfecting a cell with a nucleic acid that encodes the humanized antibody sequence, or with a nucleic acid that encodes an antigen-binding fragment of the humanized antibody sequence. In some aspects, the method further comprises culturing the cell under conditions to express the antibody or the antigen-binding fragment. In some aspects, the method even further comprises synthesizing copies of the molecule identified as having the binding properties of (i) and (ii) and, optionally (iii). The disclosure also provides an isolated molecule produced by such process(es) and a pharmaceutical composition comprising the isolated molecule and a pharmaceutically acceptable carrier.

Other features and advantages of the disclosure will become apparent from the following description of the drawing and the detailed description. It should be understood, however, that the drawing, detailed description, and the examples, while indicating embodiments of the disclosed subject matter, are given by way of illustration only, because various changes and modifications within the spirit and scope of the disclosure will become apparent from the drawing, detailed description, and the examples.

### BRIEF DESCRIPTION OF THE DRAWINGS

The novel features of the disclosure are set forth with particularity in the appended claims. A better understanding of the features and advantages of the disclosure will be obtained by reference to the following detailed description that sets forth illustrative embodiments, in which the principles of the disclosure are utilized, and the accompanying drawings of which:

FIG. 1 shows the dose response curve for the inhibition of aggregation of monomeric TTR [“Monomer”] with an antibody (Ab1) as described in Example 5.

FIG. 2 shows the sequences of the CDRs of seven murine anti-human monomer TTR antibodies prepared via the process outlined in Example 3.

FIGS. 3A-C show the selectivity of murine anti-human monomer TTR antibodies Ab3, Ab5 and Ab7, respectively, as analyzed in the screening ELISA assay described in Example 4. These three antibodies specifically bind the monomer form of human TTR with EC50s as shown in Example 11.

FIGS. 4A-C show the selectivity of murine anti-human monomer TTR antibodies Ab1. Ab2 and Ab6, respectively, as analyzed in the screening ELISA assay described in Example 4. These three antibodies specifically bind both the monomer and wild type (WT) forms of human TTR with EC50s as shown in Example 11.

FIG. 5 shows the binding of murine anti-human monomer TTR antibody Ab1 in the indirect ELISA assay described in Example 10. The antibody shows binding to a non-linear conformational epitope. The antibody did not bind to any of 10 peptides (R0010-R0017, R0021, and R0023), previously identified as epitopes of anti-human TTR antibodies, nor any of the linear regions along the monomeric interface or mutations that allow stabilization of the monomer. Therefore, the antibody appears to bind a conformational epitope within the monomeric interface.

FIGS. 6A-6B shows the sequences of the chimera murine antibodies prepared via the process outlined in Example 6.

FIG. 7 shows the VH and VL sequences of the humanized murine antibody Ab1 prepared via the process described in Example 6.

FIG. 8 shows the specificity of humanized Ab1 as measured in the indirect ELISA assay described in Example 6. The humanized antibody Ab1 retains specificity to mTTR over stabilized T119M TTR tetramer.

FIG. 9 shows the specificity of humanized Ab2 as measured in the indirect ELISA assay described in Example 6. The humanized antibody Ab2 retains specificity to mTTR over stabilized T119M TTR tetramer.

FIG. 10 shows that detection of monomeric form of human TTR is achieved by using pairs of the antibodies of the disclosure as described in Example 15. Specifically, Ab3 is used for capture and Ab5 is used for detection.

FIG. 11 shows that detection of monomeric form of human TTR is achieved by using pairs of the antibodies of the disclosure as described in Example 15. Specifically. Ab5 is used for capture and Ab3 is used for detection.

FIG. 12 shows that detection of monomeric form of human TTR is achieved with the antibodies of the disclosure as described in the TTR aggregate dot blot assay described in Example 13. Specifically. Ab1 binds human TTR monomer, but not aggregated TTR. Some binding of tryptic aggregate is seen likely due to excess of the non-aggregated monomeric form present.

FIG. 13 shows that detection of a monomeric form of human TTR is achieved with the antibodies of the disclosure as described in the TTR aggregate dot blot assay described in Example 13. Specifically, the humanized Ab1 binds human TTR monomer.

FIG. 14 shows the VH and VL sequences of the humanized murine antibody Ab2 prepared via the process described in Example 6.

FIG. 15 shows the use of the antibodies of the disclosure as capture or detection antibodies in TTR diagnostic assay described in Examples 14 and 15 using blood plasma samples from patients which have been self-declared as diagnosed with ATTR disease and either on treatment or not currently on treatment. The group on the right contains measurement of monomer levels in patients either not currently on treatment or on stabilizer treatments (tafamidis or diflunisal). The middle group is on treatments known as silencers (e.g., onpattro). The group on the right are healthy volunteers. ATTR patients showed elevated levels of monomeric TTR relative to total TTR over healthy controls.

FIG. 16 shows the binding of Ab1 to disease-relevant mutations in an Indirect ELISA assay described in Example 12.

FIG. 17 shows the binding of Ab1 to disease-relevant mutations in a Sandwich ELISA assay described in Example 12.

FIG. 18 shows the VH and VL sequences of the murine antibody Ab3 and chimeric and humanized versions of the murine antibody Ab3 prepared via the process described in Example 6.

FIG. 19 shows the VH and VL sequences of the murine antibody Ab5 and chimeric and humanized versions of the murine antibody Ab5 prepared via the process described in Example 6.

FIG. 20 shows the VH and VL sequences of the murine antibody Ab6 and chimeric and humanized versions of the murine antibody Ab6 prepared via the process described in Example 6.

FIG. 21 shows the VH and VL sequences of the humanized murine antibody murine antibody Ab7 and chimeric and humanized versions of the murine antibody Ab7 prepared via the process described in Example 6.

### DETAILED DESCRIPTION

### Definitions

An “acceptor human framework” for the purposes herein is a framework comprising the amino acid sequence of a light chain variable domain (VL) framework or a heavy chain variable domain (VH) framework derived from a human immunoglobulin framework or a human consensus framework, as defined below. An acceptor human framework “derived from” a human immunoglobulin framework or a human consensus framework may comprise the same amino acid sequence thereof, or it may contain amino acid sequence changes. In some aspects, the number of amino acid changes are 10 or less, 9 or less, 8 or less, 7 or less. 6 or less. 5 or less, 4 or less, 3 or less, or 2 or less. In some aspects, the VL acceptor human framework is identical in sequence to the VL human immunoglobulin framework sequence or human consensus framework sequence.

As used herein, the term “affinity” refers to the strength of interaction between an antibody and antigen at single antigenic sites. Within each antigenic site, the variable regions of the antibody interact through weak non-covalent forces with the antigen at numerous sites; the more interactions, the stronger the affinity. The affinity of an antibody for its antigen can generally be represented by the dissociation constant (KD). Affinity can be measured by common methods known in the art, including those described herein. Specific illustrative and exemplary methods for measuring binding affinity are described in the following. As used herein, the term “high affinity” for an antibody (such as an IgG) or fragment thereof (e.g., a Fab fragment) refers to an antibody having an affinity of about 10⁻⁷ M or less, about 10⁻⁸ M or less, about 10⁻⁹ M or less, or about 10⁻¹⁰ M or less, for a target antigen. However, high affinity binding can vary for other antibody isotypes. For example, high affinity binding for an IgM isotype refers to an antibody having an affinity of about 10-7 M or less, or about 10-8 M or less.

An “affinity matured” antibody refers to an antibody with one or more alterations in one or more complementarity determining regions (CDRs), compared to a parent antibody which does not possess such alterations, such alterations resulting in an improvement in the affinity of the antibody for antigen.

The terms “anti-mTTR antibody” and “an antibody that binds to TTR monomer” refer to an antibody that is capable of binding the monomer form of TTR, such as human TTR, with sufficient affinity such that the antibody is useful as a diagnostic and/or therapeutic agent in targeting monomeric TTR (as an intermediate to aggregated forms of the protein). In one aspect, the extent of binding of an anti-mTTR antibody to an unrelated, non-TTR protein is less than about 10% of the binding of the antibody to mTTR as measured, e.g., by ELISA Assay. In certain aspects, an antibody that binds to mTTR has a dissociation constant (KD) of about <1 μM, about <100 nM, about <10 nM, about <1 nM, about <0.1 nM, about <0.01 nM, or about <0.001 nM (e.g., about 10⁻⁸ M or less, e.g., from about 10⁻⁸ M to about 10⁻¹¹ M, e.g., from about 10⁻⁹ M to about 10⁻¹⁰ M) when measured by biolayer interferometry or indirect ELISA. An antibody is said to “specifically bind” to TTR when the antibody has a KD of about 1 μM or less. In certain aspects, an anti-TTR antibody binds to an epitope of TTR that is conserved among TTR from different species. In certain aspects, an anti-TTR antibody binds to a non-linear epitope of TTR. In certain aspects, an antibody that binds to native or tetramer form of TTR has a dissociation constant (KD) of about <10 μM, about <10 μM, about <100 nM, about <10 nM, or about <1 nM. (e.g., about 105 M or less) when measured by biolayer interferometry or indirect ELISA.

The term “antibody” herein is used in the broadest sense and encompasses various antibody structures, including but not limited to monoclonal antibodies, polyclonal antibodies, and antibody fragments, or antigen binding fragments, so long as they exhibit the desired antigen-binding activity.

An “antibody fragment” or “antigen binding fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)2; diabodies; linear antibodies; single-chain antibody molecules (e.g., scFv, and scFab); single domain antibodies (dAbs); and multispecific antibodies formed from antibody fragments. For a review of certain antibody fragments, see Holliger and Hudson, Nature Biotechnology 23:1126-1136 (2005).

A “binding domain” of an antibody, as used herein, refers to a portion of a variable domain that is sufficient to bind antigen. In some embodiments, a binding domain comprises heavy chain (HC) CDR1, CDR2, and CDR3 and light chain (LC) CDR1. CDR2, and CDR3. In some embodiments, a binding domain comprises heavy chain (HC) CDR1, FR2, CDR2, FR3, and CDR3 and light chain (LC) CDR1, FR2, CDR2, FR3, and CDR3.

The term “epitope” denotes the site on an antigen, either proteinaceous or nonproteinaceous, to which an antibody binds. Epitopes can be formed from contiguous amino acid stretches (linear epitope) or comprise non-contiguous amino acids (conformational epitope), e.g., coming in spatial proximity due to the folding of the antigen, i.e. by the tertiary folding of a proteinaceous antigen. Linear epitopes typically remain intact in denatured protein, whereas conformational epitopes are typically destroyed upon treatment with denaturing agents. An epitope comprises at least 3, at least 4, at least 5, at least 6, at least 7, or at least 8-10 amino acids in a unique spatial conformation. “Binds the same epitope as” means the ability of an antibody, antibody fragment or other antigen-binding moiety to bind to a specific antigen and binding to the same epitope as the exemplified antibody when using the same epitope mapping technique for comparing the antibodies. The epitopes of the exemplified antibody and other antibodies can be determined using epitope mapping techniques. Epitope mapping techniques are well known in the art. For example, linear epitopes can be identified by showing binding of synthetic peptide fragments derived from the antigen deposited on a membrane in the form of a dot blot, a peptide microarray, or by ELISA. Conformational epitopes can be readily identified by determining spatial conformation of amino acids such as by, e.g., hydrogen/deuterium exchange mass spectrometry or nuclear magnetic resonance, x-ray crystallography and two dimensional nuclear magnetic resonance. In certain aspects, the term “epitope” denotes a site on mTTR.

Screening for antibodies binding to a particular epitope (i.e., those binding to the same epitope) can be done using methods routine in the art such as, e.g., without limitation, alanine scanning, peptide blots (see Meth. Mol. Biol. 248 (2004) 443-463), peptide cleavage analysis, epitope excision, epitope extraction, chemical modification of antigens (see Prot. Sci. 9 (2000) 487-496), and cross-blocking (see “Antibodies”, Harlow and Lane (Cold Spring Harbor Press, Cold Spring Harbor, NY). In addition, antibodies that recognize an epitope can be identified via ELISA.

Competitive binding can be used to easily determine whether an antibody binds to the same epitope of TTR as, or competes for binding with, an anti-TTR antibody. For example, an “antibody that binds to the same epitope” as a reference anti-TTR antibody refers to an antibody that blocks binding of the reference anti-TTR antibody, respectively, to its antigen in a competition assay by 50% or more, and conversely, the reference antibody blocks binding of the antibody to its antigen in a competition assay by 50% or more. Also, to determine if an antibody binds to the same epitope as a reference anti-TTR antibody, the reference antibody is allowed to bind to TTR under saturating conditions. After removal of the excess of the reference anti-TTR antibody, the ability of an anti-TTR antibody in question to bind to TTR is assessed. If the anti-TTR antibody is able to bind to TTR after saturation binding of the reference anti-TTR antibody, it can be concluded that the anti-TTR antibody in question binds to a different epitope than the reference anti-TTR antibody. But, if the anti-TTR antibody in question is not able to bind to TTR after saturation binding of the reference anti-TTR antibody, then the anti-TTR antibody in question may bind to the same epitope as the epitope bound by the reference anti-TTR antibody. To confirm whether the antibody in question binds to the same epitope or is just hampered from binding by steric reasons routine experimentation can be used (e.g., peptide mutation and binding analyses using ELISA, RIA, surface plasmon resonance, flow cytometry or any other quantitative or qualitative antibody-binding assay available in the art). This assay should be carried out in two set-ups, i.e. with both of the antibodies being the saturating antibody. If, in both set-ups, only the first (saturating) antibody is capable of binding to TTR, then it can be concluded that the anti-TTR antibody in question and the reference anti-TTR antibody compete for binding to TTR. In some aspects, two antibodies are deemed to bind to the same or an overlapping epitope if a 1-, 5-, 10-, 20- or 100-fold excess of one antibody inhibits binding of the other by at least 50%, at least 75%, at least 90% or even 99% or more as measured in a competitive binding assay (see, e.g., Junghans et al., Cancer Res. 50 (1990) 1495-1502). In some aspects, two antibodies are deemed to bind to the same epitope if essentially all amino acid mutations in the antigen that reduce or eliminate binding of one antibody also reduce or eliminate binding of the other. Two antibodies are deemed to have “overlapping epitopes” if only a subset of the amino acid mutations that reduce or eliminate binding of one antibody reduce or eliminate binding of the other.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG1, IgG2, IgG3, IgG4, IgA1, and IgA2. In certain aspects, the antibody is of the IgG2 or IgG4 isotype. The heavy chain constant domains that correspond to the different classes of immunoglobulins are called a, d, e, g, and m, respectively. The light chain of an antibody may be assigned to one of two types, called kappa (K) and lambda (l), based on the amino acid sequence of its constant domain.

The terms “complementarity-determining region” or “CDR,” as used herein, refer to the sequences of amino acids within antibody variable regions which confer antigen specificity and binding affinity. For example, in general, there are three CDRs in each heavy chain variable region (e.g., HCDR1, HCDR2, and HCDR3) and three CDRs in each light chain variable region (LCDR1, LCDR2, and LCDR3). The precise amino acid sequence boundaries of a given CDR can be determined using any of a number of well-known schemes, including those described by Rabat et al. (1991), “Sequences of Proteins of Immunological Interest,” 5th Ed. Public Health Service, National Institutes of Health, Bethesda, MD (“Rabat” numbering scheme), Al-Lazikani et al., (1997) JMB 273,927-948 (“Chothia” numbering scheme), or a combination thereof, and ImMunoGenTics (IMGT) numbering (Lefranc, The Immunologist, 7, 132-136 (1999); Lefranc et al., Dev. Comp. Immunol., 27, 55-77 (2003); Lefranc et al., (2015) Nucleic Acids Res. 43, D413-422) (“IMGT” numbering scheme). In a combined Rabat and Chothia numbering scheme for a given CDR region (for example, HCDR1, HCDR2. HCDR3, LCDR1, LCDR2 or LCDR3), in some embodiments, the CDRs correspond to the amino acid residues that are defined as part of the Rabat CDR, together with the amino acid residues that are defined as part of the Chothia CDR. As used herein, the CDRs defined according to the “Chothia” number scheme are also sometimes referred to as “hypervariable loops.” Under IMGT, the CDR regions of an antibody can be determined using the program IMGT/DomainGap Align. Generally, unless specifically indicated, the antibody molecules can include any combination of one or more Aho CDRs, Rabat CDRs and/or Chothia CDRs.

The term “conservative sequence modifications” refers to amino acid modifications that do not significantly affect or alter the binding characteristics of the antibody or antibody fragment containing the amino acid sequence. Such conservative modifications include amino acid substitutions, additions and deletions. Modifications can be introduced into an antibody or antibody fragment of the disclosure by standard techniques known in the art, such as site-directed mutagenesis and PCR-mediated mutagenesis. Conservative amino acid substitutions are ones in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, one or more amino acid residues within an antibody or an antigen-binding fragment thereof of the disclosure can be replaced with other amino acid residues from the same side chain family and the altered antibody or antigen-binding fragment can be tested using the functional assays described herein.

An “effective amount” of an agent, e.g., an antibody, a composition, or pharmaceutical composition, refers to an amount effective, at dosages and for periods of time necessary, to achieve the desired therapeutic or prophylactic result.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g., B cell receptor); and B cell activation.

The term “Fc region”, as used herein, refers to a polypeptide comprising the CH3, CH2 and at least a portion of the hinge region of a constant domain of an antibody. Optionally, an Fc region may include a CH4 domain, present in some antibody classes. An Fc region may comprise the entire hinge region of a constant domain of an antibody. In one embodiment, the disclosure comprises an Fc region and a CH1 region of an antibody. In one embodiment, the disclosure comprises an Fc region CH3 region of an antibody. In another embodiment, the disclosure comprises an Fc region, a CH1 region and a Ckappa/lambda region from the constant domain of an antibody. In one embodiment, a binding molecule of the disclosure comprises a constant region, e.g., a heavy chain constant region. In one embodiment, such a constant region is modified compared to a wild-type constant region. That is, the polypeptides of the disclosure disclosed herein may comprise alterations or modifications to one or more of the three heavy chain constant domains (CH1, CH2 or CH3) and/or to the light chain constant region domain (CL). Example modifications include additions, deletions or substitutions of one or more amino acids in one or more domains. Such changes may be included to optimize effector function, half-life, etc. The term includes native sequence Fc regions and variant Fc regions. In one aspect, a human IgG heavy chain Fc region extends from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore, an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to EU index). Therefore, the C-terminal lysine (Lys447), or the C-terminal glycine (Gly446) and lysine (Lys447), of the Fc region may or may not be present. Thus, a “full-length IgG1” for example, includes an IgG1 with Gly446 and Lys447, or without Lys447, or without both Gly446 and Lys447. Amino acid sequences of heavy chains including an Fc region are denoted herein without C-terminal glycine-lysine dipeptide if not indicated otherwise. In one aspect, a heavy chain including an Fc region as specified herein, comprised in an antibody according to the disclosure, may comprise Gly446 and Lys447 (numbering according to EU index). In one aspect, a heavy chain including an Fc region as specified herein, comprised in an antibody according to the disclosure, may comprise Gly446 (numbering according to EU index). Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, MD, 1991.

“Framework” or “FR” refers to variable domain residues other than complementarity-determining regions (CDRs). The FR of a variable domain generally consists of four FR domains: FR1, FR2, FR3, and FR4. Accordingly, the CDR and FR sequences generally appear in the following sequence in VH (or VL): FR1-CDR-H1 (CDR-L1)-FR2-CDR-H2 (CDR-L2)-FR3-CDR-H3 (CDR-L3)-FR4.

The terms “full length antibody”, “intact antibody”, and “whole antibody” are used herein interchangeably to refer to an antibody having a structure substantially similar to a native antibody structure of having heavy chains that contain an Fc region as defined herein.

The terms “host cell”, “host cell line”, and “host cell culture” are used interchangeably and refer to cells into which exogenous nucleic acid has been introduced, including the progeny of such cells. Host cells include “transformants” and “transformed cells”, which include the primary transformed cell and progeny derived therefrom without regard to the number of passages. Progeny may not be completely identical in nucleic acid content to a parent cell, but may contain mutations. Mutant progeny that have the same function or biological activity as screened or selected for in the originally transformed cell are included herein.

A “human antibody” is one which possesses an amino acid sequence which corresponds to that of an antibody produced by a human or a human cell or derived from a non-human source that utilizes human antibody repertoires or other human antibody-encoding sequences. This definition of a human antibody specifically excludes a humanized antibody comprising non-human antigen-binding residues.

A “human consensus framework” is a framework which represents the most commonly occurring amino acid residues in a selection of human immunoglobulin VL or VH framework sequences. Generally, the selection of human immunoglobulin VL or VH sequences is from a subgroup of variable domain sequences. Generally, the subgroup of sequences is a subgroup as in Rabat et al., Sequences of Proteins of Immunological Interest, Fifth Edition, NIH Publication 91-3242, Bethesda MD (1991), vols. 1-3. In one aspect, for the VL, the subgroup is subgroup kappa I as in Rabat et al., supra. In one aspect, for the VH, the subgroup is subgroup III as in Rabat et al., supra.

A “humanized” antibody refers to a chimeric antibody comprising amino acid residues from non-human CDRs and amino acid residues from human FRs. In certain aspects, a humanized antibody will comprise substantially all of at least one, and typically two, variable domains, in which all or substantially all of the CDRs correspond to those of a non-human antibody, and all or substantially all of the FRs correspond to those of a human antibody. A humanized antibody optionally may comprise at least a portion of an antibody constant region derived from a human antibody. A “humanized form” of an antibody, e.g., a non-human antibody, refers to an antibody that has undergone humanization.

An “individual” or “subject” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain aspects, the individual or subject is a human.

An “isolated” antibody is one which has been separated from a component of its natural environment. In some aspects, an antibody is purified to greater than 95% or 99% purity as determined by, for example, electrophoretic (e.g., SDS-PAGE, isoelectric focusing (IEF), capillary electrophoresis) or chromatographic (e.g., ion exchange or reverse phase HPLC) methods. For a review of methods for assessment of antibody purity, see, e.g., Flatman et al., J. Chromatogr. B 848:79-87 (2007).

The term “linked” when used in the context of two polypeptides, means that the polypeptides are part of the same sequence of amino acids. Two polypeptides that are linked may be separated by additional amino acid sequence; that is, they need not be contiguous or directly linked to one another.

An “isolated” nucleic acid refers to a nucleic acid molecule that has been separated from a component of its natural environment. An isolated nucleic acid includes a nucleic acid molecule contained in cells that ordinarily contain the nucleic acid molecule, but the nucleic acid molecule is present extra-chromosomally or at a chromosomal location that is different from its natural chromosomal location.

“Isolated nucleic acid encoding an anti-TTR antibody” refers to one or more nucleic acid molecules encoding anti-TTR antibody heavy and light chains (or fragments thereof), including such nucleic acid molecule(s) in a single vector or separate vectors, and such nucleic acid molecule(s) present at one or more locations in a host cell.

The term “monoclonal antibody”, as used herein, refers to an antibody obtained from a population of substantially homogeneous antibodies, i.e., the individual antibodies comprising the population are identical and/or bind the same epitope, except for possible variant antibodies, e.g., containing naturally occurring mutations or arising during production of a monoclonal antibody preparation, such variants generally being present in minor amounts. In contrast to polyclonal antibody preparations, which typically include different antibodies directed against different determinants (epitopes), each monoclonal antibody of a monoclonal antibody preparation is directed against a single determinant on an antigen. Thus, the modifier “monoclonal” indicates the character of the antibody as being obtained from a substantially homogeneous population of antibodies, and is not to be construed as requiring production of the antibody by any particular method. For example, the monoclonal antibodies in accordance with the disclosure may be made by a variety of techniques, including but not limited to the hybridoma method, recombinant DNA methods, phage-display methods, and methods utilizing transgenic animals containing all or part of the human immunoglobulin loci, such methods and other exemplary methods for making monoclonal antibodies being described herein.

A “naked antibody” refers to an antibody that is not conjugated to a heterologous moiety (e.g., a cytotoxic moiety) or radiolabel. The naked antibody may be present in a pharmaceutical composition.

“Native antibodies” refer to naturally occurring immunoglobulin molecules with varying structures. For example, native IgG antibodies are hetero-tetrameric glycoproteins of about 150,000 daltons, composed of two identical light chains and two identical heavy chains that are disulfide-bonded. From N- to C-terminus, each heavy chain has a variable domain (VH), also called a variable heavy domain or a heavy chain variable region, followed by three constant heavy domains (CH1, CH2, and CH3). Similarly, from N- to C-terminus, each light chain has a variable domain (VL), also called a variable light domain or a light chain variable region, followed by a constant light (CL) domain.

The term “nucleic acid” or “nucleic acid molecule” or “polynucleotide” includes any compound and/or substance that comprises a polymer of nucleotides. Each nucleotide is composed of a base, specifically a purine- or pyrimidine base (i.e. cytosine (C), guanine (G), adenine (A), thymine (T) or uracil (U)), a sugar (i.e. deoxyribose or ribose), and a phosphate group. Often, the nucleic acid molecule is described by the sequence of bases, whereby said bases represent the primary structure (linear structure) of a nucleic acid molecule. The sequence of bases is typically represented from 5′ to 3′. Herein, the term nucleic acid molecule encompasses deoxyribonucleic acid (DNA) including e.g., complementary DNA (cDNA) and genomic DNA, ribonucleic acid (RNA), in particular, messenger RNA (mRNA), synthetic forms of DNA or RNA, and mixed polymers comprising two or more of these molecules. The nucleic acid molecule may be linear or circular. In addition, the term nucleic acid molecule includes both, sense and antisense strands, as well as single stranded and double stranded forms. Moreover, the herein described nucleic acid molecule can contain naturally occurring or non-naturally occurring nucleotides. Examples of non-naturally occurring nucleotides include modified nucleotide bases with derivatized sugars or phosphate backbone linkages or chemically modified residues. Nucleic acid molecules also encompass DNA and RNA molecules which are suitable as a vector for direct expression of an antibody of the disclosure in vitro and/or in vivo, e.g., in a host or patient. Such DNA (e.g., cDNA) or RNA (e.g., mRNA) vectors, can be unmodified or modified. For example, mRNA can be chemically modified to enhance the stability of the RNA vector and/or expression of the encoded molecule so that mRNA can be injected into a subject to generate the antibody in vivo (see e.g., Stadler et al, Nature Medicine 2017, published online 12 Jun. 2017. doi: 10.1038/nm.4356 or EP 2 101 823 BI).

The term “package insert” is used to refer to instructions customarily included in commercial packages of therapeutic products, that contain information about the indications, usage, dosage, administration, combination therapy, contraindications and/or warnings concerning the use of such therapeutic products.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. A polypeptide includes a natural peptide, a recombinant peptide, or a combination thereof.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity for the purposes of the alignment. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Unless otherwise indicated, for purposes herein, percent amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et al. (1997) Genomics 46:24-36, and is publicly available from www.fasta.bioch.virginia.edu/fasta_www2/fasta_down.shtml or www. ebi.ac.uk/Tools/sss/fasta. Alternatively, a public server accessible at fasta.bioch.virginia.edu/fasta_www2/index.cgi can be used to compare the sequences, using the ggsearch (global protein: protein) program and default options (BLOSUM50; open: −10; ext: −2; Ktup=2) to ensure a global, rather than local, alignment is performed. Percent amino acid identity is given in the output alignment header.

The term “pharmaceutical composition” or “pharmaceutical formulation” refers to a preparation which is in such form as to permit the biological activity of an active ingredient contained therein to be effective, and which contains no additional components which are unacceptably toxic to a subject to which the composition or pharmaceutical composition is administered.

A “pharmaceutically acceptable carrier” refers to an ingredient in a composition or a pharmaceutical composition or formulation, other than an active ingredient, which is nontoxic to a subject. A pharmaceutically acceptable carrier includes, but is not limited to, a buffer, excipient, stabilizer, or preservative.

The term “specifically binds” or “binding specificity” as used herein refers to the ability of an individual antibody combining site to react with one antigenic determinant and not with a different antigenic determinant. The combining site of the antibody is located in the Fab portion of the molecule and is constructed from the hypervariable regions of the heavy and light chains. Binding affinity of an antibody is the strength of the reaction between a single antigenic determinant and a single combining site on the antibody. It is the sum of the attractive and repulsive forces operating between the antigenic determinant and the combining site of the antibody.

The term “TTR,” as used herein, refers to any native transthyretin (e.g., SEQ ID NO: 1) from any vertebrate source, including mammals such as primates (e.g., humans) and rodents (e.g., mice and rats), unless otherwise indicated. The term encompasses “full-length”, unprocessed TTR as well as any post-translational form that results from processing in the cell. Such truncated TTR forms can result from protease cleavage. The term also encompasses naturally occurring variants of TTR. e.g., splice variants or allelic variants. The amino acid sequence of an exemplary human mature TTR protein is shown in SEQ ID NO: 1. The mutations noted herein are numbered according the mature human TTR sequence set out in SEQ ID NO: 1.

The term “TTR forms,” or “forms of TTR”, as used herein, refers to the structural forms of the protein such as different quaternary structures referred to as the monomeric form of TTR (“mTTR’) as well as the tetrameric form of TTR. See A W Yee, et al., Nature Communications, volume 10, Article number: 925 (2019), and Quintas et al., J. BIOLOGICAL CHEMISTRY, Vol. 274, No. 46, pp. 32943-32949, 1999. It also includes folding intermediates.

The term “mTTR” refers to the monomeric form of TTR. The monomeric form can be in the “stabilized monomer” form. This stabilized monomer form can prevent the conversion into fibrils or aggregates. Stabilized mTTR's include the Phe87Met mutation (SEQ ID NO: 143) or the Leu110Met TTR mutation (SEQ ID NO: 144) or the Phe87Met Leu110Met (F87M L110M) TTR double mutation (SEQ ID NO: 80).

The term “dTTR” refers to the dimeric form of TTR, this can be in the form of a “stabilized dimer” form. This stabilized dimer cannot readily form a tetramer and includes the Ser112Ile (S112I) TTR mutation.

The term “TTR folding intermediates,” “folding intermediates,” or “intermediates” of TTR as used herein, refers to partially folded TTR forms, such as the TTR tetramer dissociation or the refolding of monomers. See Feige et al., PNAS, Sep. 9, 2008, 105 (36). 13373-13378; or Jesus et al., Int J Mol Sci., 2016 September; 17(9): 1428.

The term “TTR aggregation” refers to the extracellular misfolding and/or misassembly into a spectrum of aggregate structures. One of those can be an ordered aggregate that is defined by the characteristic cross-β-sheet assemblies called amyloid fibrils which are one of the structures formed from the process of aggregation or amyloidogenesis. This aggregation is thought to cause degenerative diseases referred to as amyloid diseases. See Schmidt, M., Wiese, S., Adak, V. et al. Cryo-EM structure of a transthyretin-derived amyloid fibril from a patient with hereditary ATTR amyloidosis. Nat Commun 10, 5008 (2019). Such aggregates can be an ordered aggregate or amyloid. Amyloid may also be amorphous.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology (“treating or ameliorating a disease”). Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some aspects, antibodies of the disclosure are used to delay development of a disease or to slow the progression of a disease.

In some aspects, therefore, the disclosure provides antibodies for treating a disease or for development of a medicament for treating a disease associated with TTR aggregation. In some aspects, the disease associated with TTR aggregation is TTR amyloidosis. In some aspects, the TTR amyloidosis is transthyretin amyloidosis (ATTR) cardiomyopathy (ATTR-CM) or transthyretin amyloidosis (ATTR) polyneuropathy (ATTR-PN). In some aspects, the disease associated with TTR aggregation is peripheral TTR amyloidosis, ocular amyloid angiopathy or cerebral amyloid angiopathy. In some aspects, the disease associated with TTR aggregation is Familial Amyloid Polyneuropathy (AP), Familial Amyloid Cardiomyopathy (FAC), ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia. TTR-related ligament amyloidosis including carpal tunnel syndrome, rotator cuff tears and lumbar spinal stenosis, or preeclampsia.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three complementarity determining regions (CDRs). See, e.g., Kindt et al. Kuby Immunology, 6th ed., W.H. Freeman and Co., page 91 (2007). A variable domain may comprise heavy chain (HC) CDR1-FR2-CDR2-FR3-CDR3 with or without all or a portion of FR1 and/or FR4; and light chain (LC) CDR1-FR2-CDR2-FR3-CDR3 with or without all or a portion of FR1 and/or FR4. That is, a variable domain may lack a portion of FR1 and/or FR4 so long as it retains antigen-binding activity. A single VH or VL domain may be sufficient to confer antigen-binding specificity. Furthermore, antibodies that bind a particular antigen may be isolated using a VH or VL domain from an antibody that binds the antigen to screen a library of complementary VL or VH domains, respectively. See, e.g., Portolano et al., Immunol. 150:880-887 (1993); Clarkson et al., Nature 352:624-628 (1991).

The term “vector”, as used herein, refers to a nucleic acid molecule capable of propagating another nucleic acid to which it is linked. The term includes the vector as a self-replicating nucleic acid structure as well as the vector incorporated into the genome of a host cell into which it has been introduced. Certain vectors are capable of directing the expression of nucleic acids to which they are operatively linked. Such vectors are referred to herein as “expression vectors”.

### Compositions and Methods

In one aspect, the disclosure is based, in part, on the finding that antibodies can bind to the monomer form of TTR. In certain aspects, antibodies that specifically bind the monomer form of TTR are provided. Antibodies of the disclosure are useful, such as for treatment of TTR related diseases as well as in diagnostic tests.

In one aspect, the antibodies can bind to the TTR intermediates or folding intermediates.

In certain aspects, an anti-mTTR antibody inhibits TTR aggregation in vitro or in vivo. In some embodiments, TTR aggregation is inhibited at an antibody concentration of less than about 200 nM.

In some embodiments, an anti-TTR antibody binds human TTR with a KD of less than about 20 nM, less than about 10 nM, less than about 5 nM, or less than about 2 nM, or less than about 1 nM, as measured by biolayer interferometry.

In some embodiments, an anti-TTR antibody binds tetrameric human TTR, with an EC₅₀ of no greater than about 20 nM, or no greater than about 10 nM, or no greater than about 5 nM, or no greater than about 1 nM, as measured by indirect ELISA.

In one aspect, the disclosure provides an anti-TTR antibody comprising at least one, at least two, at least three, at least four, at least five, or all six CDRs selected from (a) CDR-H1 comprising the amino acid sequence of SEQ ID Nos 2-8; (b) CDR-H2 comprising the amino acid sequence of SEQ ID Nos 9-15; (c) CDR-H3 comprising the amino acid sequence of SEQ ID Nos 16-22; and a light chain variable domain (VL) comprising (d) CDR-L1 comprising the amino acid sequence of SEQ ID Nos 23-29; (e) CDR-L2 comprising the amino acid sequence of SEQ ID Nos 30-36; and (f) CDR-L3 comprising the amino acid sequence of SEQ ID Nos 37-43.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 2; (b) CDR-H2 comprising the amino acid sequence of SEQ ID NO: 9; (c) CDR-H3 comprising the amino acid sequence of SEQ ID NO: 16; (d) CDR-L1 comprising the amino acid sequence of SEQ ID NO: 23; (e) CDR-L2 comprising the amino acid sequence of SEQ ID NO: 30; and (f) CDR-L3 comprising the amino acid sequence of SEQ ID NO: 37.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 3; (b) CDR-H2 comprising the amino acid sequence of SEQ ID NO: 10; (c) CDR-H3 comprising the amino acid sequence of SEQ ID NO: 17; (d) CDR-L1 comprising the amino acid sequence of SEQ ID NO: 24; (e) CDR-L2 comprising the amino acid sequence of SEQ ID NO: 31; and (f) CDR-L3 comprising the amino acid sequence of SEQ ID NO: 38.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 4; (b) CDR-H2 comprising the amino acid sequence of SEQ ID NO: 11; (c) CDR-H3 comprising the amino acid sequence of SEQ ID NO: 18; (d) CDR-L1 comprising the amino acid sequence of SEQ ID NO: 25; (e) CDR-L2 comprising the amino acid sequence of SEQ ID NO: 32; and (f) CDR-L3 comprising the amino acid sequence of SEQ ID NO: 39.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 5; (b) CDR-H2 comprising the amino acid sequence of SEQ ID NO: 12; (c) CDR-H3 comprising the amino acid sequence of SEQ ID NO: 19; (d) CDR-L1 comprising the amino acid sequence of SEQ ID NO: 26; (e) CDR-L2 comprising the amino acid sequence of SEQ ID NO: 33; and (f) CDR-L3 comprising the amino acid sequence of SEQ ID NO: 40.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 6; (b) CDR-H2 comprising the amino acid sequence of SEQ ID NO: 13; (c) CDR-H3 comprising the amino acid sequence of SEQ ID NO: 20: (d) CDR-L1 comprising the amino acid sequence of SEQ ID NO: 27; (e) CDR-L2 comprising the amino acid sequence of SEQ ID NO: 34; and (f) CDR-L3 comprising the amino acid sequence of SEQ ID NO: 41.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 7; (b) CDR-H2 comprising the amino acid sequence of SEQ ID NO: 14; (c) CDR-H3 comprising the amino acid sequence of SEQ ID NO: 21; (d) CDR-L1 comprising the amino acid sequence of SEQ ID NO: 28; (e) CDR-L2 comprising the amino acid sequence of SEQ ID NO: 35; and (f) CDR-L3 comprising the amino acid sequence of SEQ ID NO: 42.

In one aspect, the disclosure provides an anti-TTR antibody comprising at least one, at least two, at least three, at least four, at least five, or all six CDRs selected from (a) CDR-H1 comprising the amino acid sequence of SEQ ID NO: 8; (b) CDR-H2 comprising the amino acid sequence of SEQ ID NO: 15; (c) CDR-H3 comprising the amino acid sequence of SEQ ID NO: 22; (d) CDR-L1 comprising the amino acid sequence of SEQ ID NO: 29; (e) CDR-L2 comprising the amino acid sequence of SEQ ID NO: 36; and (f) CDR-L3 comprising the amino acid sequence of SEQ ID NO: 43.

In one aspect, the disclosure provides an antibody comprising at least one, at least two, or all three VH CDR sequences selected from (a) CDR-H1 comprising the amino acid sequence of SEQ ID NOs: 2-8; (b) CDR-H2 comprising the amino acid sequence of SEQ ID NOs: 9-15; and (c) CDR-H3 comprising the amino acid sequence of SEQ ID NOs: 16-22.

In one aspect, the antibody comprises CDR-H3 comprising the amino acid sequence of SEQ ID NOs: 16-22. In another aspect, the antibody comprises CDR-H3 comprising the amino acid sequence of SEQ ID NOs: 16-22 and CDR-L3 comprising the amino acid sequence of SEQ ID NOs: 37-43. In a further aspect, the antibody comprises CDR-H3 comprising the amino acid sequence of SEQ ID NOs: 16-22, CDR-L3 comprising the amino acid sequence of SEQ ID NOs: 37-43, and CDR-H2 comprising the amino acid sequence of SEQ ID NOs: 9-15.

In one aspect, the antibody comprises CDR-H3 comprising the amino acid sequence of SEQ ID NO: 16. In another aspect, the antibody comprises CDR-H3 comprising the amino acid sequence of SEQ ID NO: 16 and CDR-L3 comprising the amino acid sequence of SEQ ID NO: 37. In a further aspect, the antibody comprises CDR-H3 comprising the amino acid sequence of SEQ ID NO: 16. CDR-L3 comprising the amino acid sequence of SEQ ID NO: 37, and CDR-H2 comprising the amino acid sequence of SEQ ID NO: 9.

In one aspect, the antibody comprises CDR-H3 comprising the amino acid sequence of SEQ ID NO: 17. In another aspect, the antibody comprises CDR-H3 comprising the amino acid sequence of SEQ ID NO: 17 and CDR-L3 comprising the amino acid sequence of SEQ ID NOs: 38. In a further aspect, the antibody comprises CDR-H3 comprising the amino acid sequence of SEQ ID NO: 17. CDR-L3 comprising the amino acid sequence of SEQ ID NO: 38, and CDR-H2 comprising the amino acid sequence of SEQ ID NO: 10.

In one aspect, the antibody comprises CDR-H3 comprising the amino acid sequence of SEQ ID NO: 18. In another aspect, the antibody comprises CDR-H3 comprising the amino acid sequence of SEQ ID NO: 18 and CDR-L3 comprising the amino acid sequence of SEQ ID NO: 39. In a further aspect, the antibody comprises CDR-H3 comprising the amino acid sequence of SEQ ID NO: 18, CDR-L3 comprising the amino acid sequence of SEQ ID NO: 39, and CDR-H2 comprising the amino acid sequence of SEQ ID NO: 11.

In one aspect, the antibody comprises CDR-H3 comprising the amino acid sequence of SEQ ID NO: 19. In another aspect, the antibody comprises CDR-H3 comprising the amino acid sequence of SEQ ID NO: 19 and CDR-L3 comprising the amino acid sequence of SEQ ID NO: 40. In a further aspect, the antibody comprises CDR-H3 comprising the amino acid sequence of SEQ ID NO: 19, CDR-L3 comprising the amino acid sequence of SEQ ID NO: 40, and CDR-H2 comprising the amino acid sequence of SEQ ID NO: 12.

In one aspect, the antibody comprises CDR-H3 comprising the amino acid sequence of SEQ ID NO: 20. In another aspect, the antibody comprises CDR-H3 comprising the amino acid sequence of SEQ ID NO: 20 and CDR-L3 comprising the amino acid sequence of SEQ ID NO: 41. In a further aspect, the antibody comprises CDR-H3 comprising the amino acid sequence of SEQ ID NO: 20, CDR-L3 comprising the amino acid sequence of SEQ ID NO: 41, and CDR-H2 comprising the amino acid sequence of SEQ ID NO: 13.

In one aspect, the antibody comprises CDR-H3 comprising the amino acid sequence of SEQ ID NO: 21. In another aspect, the antibody comprises CDR-H3 comprising the amino acid sequence of SEQ ID NO: 21 and CDR-L3 comprising the amino acid sequence of SEQ ID NO: 42. In a further aspect, the antibody comprises CDR-H3 comprising the amino acid sequence of SEQ ID NO: 21. CDR-L3 comprising the amino acid sequence of SEQ ID NO: 42, and CDR-H2 comprising the amino acid sequence of SEQ ID NO: 14.

In one aspect, the antibody comprises CDR-H3 comprising the amino acid sequence of SEQ ID NO: 22. In another aspect, the antibody comprises CDR-H3 comprising the amino acid sequence of SEQ ID NO: 22 and CDR-L3 comprising the amino acid sequence of SEQ ID NO: 43. In a further aspect, the antibody comprises CDR-H3 comprising the amino acid sequence of SEQ ID NO: 22, CDR-L3 comprising the amino acid sequence of SEQ ID NO: 43, and CDR-H2 comprising the amino acid sequence of SEQ ID NO: 15.

In a further aspect, the antibody comprises (a) CDR-H1 comprising the amino acid sequence of SEQ ID NO: 2; (b) CDR-H2 comprising the amino acid sequence of SEQ ID NO: 9; and (c) CDR-H3 comprising the amino acid sequence of SEQ ID NO: 16.

In a further aspect, the antibody comprises (a) CDR-H1 comprising the amino acid sequence of SEQ ID NO: 3; (b) CDR-H2 comprising the amino acid sequence of SEQ ID NO: 10; and (c) CDR-H3 comprising the amino acid sequence of SEQ ID NO: 17.

In a further aspect, the antibody comprises (a) CDR-H1 comprising the amino acid sequence of SEQ ID NO: 4; (b) CDR-H2 comprising the amino acid sequence of SEQ ID NO: 11; and (c) CDR-H3 comprising the amino acid sequence of SEQ ID NO: 18.

In a further aspect, the antibody comprises (a) CDR-H1 comprising the amino acid sequence of SEQ ID NO: 5; (b) CDR-H2 comprising the amino acid sequence of SEQ ID NO: 12; and (c) CDR-H3 comprising the amino acid sequence of SEQ ID NO: 19.

In a further aspect, the antibody comprises (a) CDR-H1 comprising the amino acid sequence of SEQ ID NO: 6; (b) CDR-H2 comprising the amino acid sequence of SEQ ID NO: 13; and (c) CDR-H3 comprising the amino acid sequence of SEQ ID NO: 20.

In a further aspect, the antibody comprises (a) CDR-H1 comprising the amino acid sequence of SEQ ID NO: 7; (b) CDR-H2 comprising the amino acid sequence of SEQ ID NO: 14; and (c) CDR-H3 comprising the amino acid sequence of SEQ ID NO: 21.

In a further aspect, the antibody comprises (a) CDR-H1 comprising the amino acid sequence of SEQ ID NO: 8; (b) CDR-H2 comprising the amino acid sequence of SEQ ID NO: 15; and (c) CDR-H3 comprising the amino acid sequence of SEQ ID NO: 22.

In another aspect, the disclosure provides an antibody comprising at least one, at least two, or all three VL CDR sequences selected from (a) CDR-L1 comprising the amino acid sequence of SEQ ID NOs: 23-29; (b) CDR-L2 comprising the amino acid sequence of SEQ ID NOs: 30-36; and (c) CDR-L3 comprising the amino acid sequence of SEQ ID NOs: 37-43.

In one aspect, the antibody comprises (a) CDR-L1 comprising the amino acid sequence of SEQ ID NO: 23; (b) CDR-L2 comprising the amino acid sequence of SEQ ID NO: 30; and (c) CDR-L3 comprising the amino acid sequence of SEQ ID NO: 37.

In one aspect, the antibody comprises (a) CDR-L1 comprising the amino acid sequence of SEQ ID NO: 24; (b) CDR-L2 comprising the amino acid sequence of SEQ ID NO: 31; and (c) CDR-L3 comprising the amino acid sequence of SEQ ID NO: 38.

In one aspect, the antibody comprises (a) CDR-L1 comprising the amino acid sequence of SEQ ID NO: 25; (b) CDR-L2 comprising the amino acid sequence of SEQ ID NO: 32; and (c) CDR-L3 comprising the amino acid sequence of SEQ ID NO: 39.

In one aspect, the antibody comprises (a) CDR-L1 comprising the amino acid sequence of SEQ ID NO: 26; (b) CDR-L2 comprising the amino acid sequence of SEQ ID NO: 33; and (c) CDR-L3 comprising the amino acid sequence of SEQ ID NO: 40.

In one aspect, the antibody comprises (a) CDR-L1 comprising the amino acid sequence of SEQ ID NO: 27; (b) CDR-L2 comprising the amino acid sequence of SEQ ID NO: 34; and (c) CDR-L3 comprising the amino acid sequence of SEQ ID NO: 41.

In one aspect, the antibody comprises (a) CDR-L1 comprising the amino acid sequence of SEQ ID NO: 28: (b) CDR-L2 comprising the amino acid sequence of SEQ ID NO: 35; and (c) CDR-L3 comprising the amino acid sequence of SEQ ID NO: 42.

In one aspect, the antibody comprises (a) CDR-L1 comprising the amino acid sequence of SEQ ID NO: 29; (b) CDR-L2 comprising the amino acid sequence of SEQ ID NO: 36; and (c) CDR-L3 comprising the amino acid sequence of SEQ ID NO: 43.

In another aspect, an antibody of the disclosure comprises (a) a VH domain comprising at least one, at least two, or all three VH CDR sequences selected from (i) CDR-H1 comprising the amino acid sequence of SEQ ID NOs: 2-8, (ii) CDR-H2 comprising the amino acid sequence of SEQ ID NOs: 9-15, and (iii) CDR-H3 comprising the amino acid sequence of SEQ ID NOs: 16-22; and (b) a VL domain comprising at least one, at least two, or all three VL CDR sequences selected from (i) CDR-L1 comprising the amino acid sequence of SEQ ID NOs: 23-29, (ii) CDR-L2 comprising the amino acid sequence of SEQ ID NO: 30-36, and (c) CDR-L3 comprising the amino acid sequence of SEQ ID NO: 37-43.

In another aspect, the disclosure provides an antibody comprising (a) CDR-H1 comprising the amino acid sequence of SEQ ID NO: 2; (b) CDR-H2 comprising the amino acid sequence of SEQ ID NO: 9; (c) CDR-H3 comprising the amino acid sequence of SEQ ID NO: 16; (d) CDR-L1 comprising the amino acid sequence of SEQ ID NO: 23; (e) CDR-L2 comprising the amino acid sequence of SEQ ID NO: 30; and (f) CDR-L3 comprising the amino acid sequence of SEQ ID NO: 37.

In another aspect, the disclosure provides an antibody comprising (a) CDR-H1 comprising the amino acid sequence of SEQ ID NO: 3; (b) CDR-H2 comprising the amino acid sequence of SEQ ID NO: 10; (c) CDR-H3 comprising the amino acid sequence of SEQ ID NO: 17: (d) CDR-L1 comprising the amino acid sequence of SEQ ID NO: 24: (e) CDR-L2 comprising the amino acid sequence of SEQ ID NO: 31; and (f) CDR-L3 comprising the amino acid sequence of SEQ ID NO: 38.

In another aspect, the disclosure provides an antibody comprising (a) CDR-H1 comprising the amino acid sequence of SEQ ID NO: 4; (b) CDR-H2 comprising the amino acid sequence of SEQ ID NO: 11; (c) CDR-H3 comprising the amino acid sequence of SEQ ID NO: 18; (d) CDR-L1 comprising the amino acid sequence of SEQ ID NO: 25; (e) CDR-L2 comprising the amino acid sequence of SEQ ID NO: 32; and (f) CDR-L3 comprising the amino acid sequence of SEQ ID NO: 39.

In another aspect, the disclosure provides an antibody comprising (a) CDR-H1 comprising the amino acid sequence of SEQ ID NO: 5; (b) CDR-H2 comprising the amino acid sequence of SEQ ID NO: 12; (c) CDR-H3 comprising the amino acid sequence of SEQ ID NO: 19; (d) CDR-L1 comprising the amino acid sequence of SEQ ID NO: 26; (e) CDR-L2 comprising the amino acid sequence of SEQ ID NO: 33; and (f) CDR-L3 comprising the amino acid sequence of SEQ ID NO: 40.

In another aspect, the disclosure provides an antibody comprising (a) CDR-H1 comprising the amino acid sequence of SEQ ID NO: 6; (b) CDR-H2 comprising the amino acid sequence of SEQ ID NO: 13; (c) CDR-H3 comprising the amino acid sequence of SEQ ID NO: 20: (d) CDR-L1 comprising the amino acid sequence of SEQ ID NO: 27: (e) CDR-L2 comprising the amino acid sequence of SEQ ID NO: 34; and (f) CDR-L3 comprising the amino acid sequence of SEQ ID NO: 41.

In another aspect, the disclosure provides an antibody comprising (a) CDR-H1 comprising the amino acid sequence of SEQ ID NO: 7: (b) CDR-H2 comprising the amino acid sequence of SEQ ID NO: 14; (c) CDR-H3 comprising the amino acid sequence of SEQ ID NO: 21; (d) CDR-L1 comprising the amino acid sequence of SEQ ID NO: 28; (e) CDR-L2 comprising the amino acid sequence of SEQ ID NO: 35; and (f) CDR-L3 comprising the amino acid sequence of SEQ ID NO: 42.

In another aspect, the disclosure provides an antibody comprising (a) CDR-H1 comprising the amino acid sequence of SEQ ID NO: 8; (b) CDR-H2 comprising the amino acid sequence of SEQ ID NO: 15; (c) CDR-H3 comprising the amino acid sequence of SEQ ID NO: 22; (d) CDR-L1 comprising the amino acid sequence of SEQ ID NO: 29; (e) CDR-L2 comprising the amino acid sequence of SEQ ID NO: 36; and (f) CDR-L3 comprising the amino acid sequence of SEQ ID NO: 43.

In another aspect, the disclosure provides an antibody comprising a CDR-H1's found in Table 1:

TABLE 1

| | Sequence | SEQ ID NO |
|---|---|---|
| CDR-H1 | GYTFTSYW | 2 |
| CDR-H1 | GFTFSNYW | 3 |
| CDR-H1 | GYTFTDYY | 4 |
| CDR-H1 | GYTFTTYG | 5 |
| CDR-H1 | GFNIKDYY | 6 |
| CDR-H1 | GYTFTGYW | 7 |
| CDR-H1 | GFNIKDYY | 8 |

In another aspect, the disclosure provides an antibody comprising a CDR-H2's found in Table 2:

TABLE 2

| | Sequence | SEQ ID NO |
|---|---|---|
| CDR-H2 | IDPSDSET | 9 |
| CDR-H2 | IRLKSDNYAT | 10 |
| CDR-H2 | INPYNGGT | 11 |
| CDR-H2 | INTYSGVP | 12 |
| CDR H2 | IDPEDGET | 13 |
| CDR H2 | ILPGSGST | 14 |
| CDR H2 | IDPEDGET | 15 |

In another aspect, the disclosure provides an antibody comprising a CDR-H3's found in Table 3:

TABLE 3

| | Sequence | SEQ ID NO |
|---|---|---|
| CDR-H3 | ARGRNYWYFDV | 16 |
| CDR-H3 | TGVFTY | 17 |
| CDR-H3 | ARDLTGTGGAMDY | 18 |
| CDR-H3 | AFYYDYQEWFAY | 19 |
| CDR-H3 | ALLWLRRGRGY | 20 |
| CDR-H3 | ARLRGSSYPWYFDY | 21 |
| CDR-H3 | ARGKTPFAY | 22 |

In another aspect, the disclosure provides an antibody comprising a CDR-L1's found in Table 4:

TABLE 4

| | Sequence | SEQ ID NO |
|---|---|---|
| CDR-L1 | ESVDNYGISF | 23 |
| CDR-L1 | ESVDSYGNSF | 24 |
| CDR-L1 | SSVSY | 25 |
| CDR-L1 | ENIYSN | 26 |
| CDR-L1 | QEISGY | 27 |
| CDR-L1 | GYTFTGYW | 28 |
| CDR-L1 | ENIYSN | 29 |

In another aspect, the disclosure provides an antibody comprising a CDR-L2's found in Table 5:

TABLE 5

| | Sequence | SEQ ID NO |
|---|---|---|
| CDR-L2 | AAS | 30 |
| CDR-L2 | LAS | 31 |
| CDR-L2 | ATS | 32 |
| CDR-L2 | AAT | 33 |
| CDR L2 | AAS | 34 |
| CDR L2 | AAT | 35 |
| CDR L2 | AAS | 36 |

In another aspect, the disclosure provides an antibody comprising a CDR-H3's found in Table 6:

TABLE 6

| | Sequence | SEQ ID NO |
|---|---|---|
| CDR-L3 | QQSKEVPWT | 37 |
| CDR-L3 | QQNNEDPMYT | 38 |
| CDR-L3 | QQWSSNPLT | 39 |
| CDR-L3 | QHFWGTPR | 40 |
| CDR L3 | LQYASYPPT | 41 |
| CDR L3 | QHFWGTPYT | 42 |
| CDR L3 | LQYDSYPFT | 43 |

In another aspect, the disclosure provides an antibody comprising a CDR-H1 comprising the amino acid sequence of Formula I:

(SEQ ID NO: 44)
Gly-X₁-X₂-X₃-X₄-X₅-Tyr-X₆

Wherein X¹ is Tyr or Phe;
   X² is Thr or Asn;
   X³ is Phe or Ile;
   X⁴ is Thr, Ser or Lys;
   X⁵ is selected from Asp, Asn, Thr, Ser and Gly; and
   X⁶ is Trp Tyr or Gly.

In another aspect, the disclosure provides an anti-mTTR antibody Ab1 comprising a heavy chain variable domain (VH) comprising the sequence

(SEQ ID NO: 45)
QVQLQQPGAELVRPGSSVKLSCKASGYTFTSYWMHWVKQRPIQGLEWIG
NIDPSDSETHYNQKFKDKATLTVDKSSSTAYMQLSSLTSEDSAVYYCAR
GRNYWYFDVWGTGTTVTVSS.

In another aspect, the disclosure provides an anti-mTTR antibody comprising a heavy chain variable domain (VH) comprising a sequence selected from

(SEQ ID NO: 46)
QVQLVQSGAEVKKPGASVKVSCKASGYTFTSYWMHWVRQAPGQGLEWMG
NIDPSDSETHYAQKFQGRVTMTRDTSTSTVYMELSSLRSEDTAVYYCAR
GRNYWYFDVWGTGTMVTVSS;
(SEQ ID NO: 47)
QVQLVQSGAEVKKPGASVKVSCKASGYTFTSYWMHWVRQAPGQGLEWMG
NIDPSDSETHYAQKFQGRVTMTVDTSTSTVYMELSSLRSEDTAVYYCAR
GRNYWYFDVWGTGTMVTVSS;
(SEQ ID NO: 48)
QVQLVQSGAEVKKPGASVKVSCKASGYTFTSYWMHWVRQAPGQGLEWMG
NIDPSDSETHYAQKFQGRVTMTVDKSTSTVYMELSSLRSEDTAVYYCAR
GRNYWYFDVWGTGTMVTVSS;
and
(SEQ ID NO: 49)
QVQLVQSGAEVKKPGASVKVSCKASGYTFTSYWMHWVRQAPGQGLEWIG
NIDPSDSETHYAQKFQGRATLTVDKSTSTVYMELSSLRSEDTAVYYCAR
GRNYWYFDVWGTGTMVTVSS.

In another aspect, the disclosure provides an anti-mTTR antibody Ab1 comprising a light chain variable domain (VL) comprising the sequence

(SEQ ID NO: 50)
DIVLTQSPASLAVSLGQRATISCRASESVDNYGISFMNWFQQKPGQPPK
LLIYAASNQGSGVPARFSGSGSGTDFSLNIHPMEEDDTAMYFCQQSKEV
PWTFGGGTKLEIK.

In another aspect, the disclosure provides a humanized anti-mTTR antibody comprising a light chain variable domain (VL) comprising the sequence selected from

(SEQ ID NO: 51)
DIQMTQSPSSLSASVGDRVTITCRASESVDNYGISFMNWYQQKPGKAPK
LLIYAASNQGSGVPSRFSGSGSGTDFTLTISSLQPEDFATYYCQQSKEV
PWTFGGGTKLEIK;

(SEQ ID NO: 52)
DIQMTQSPSSLSASVGDRVTITCRASESVDNYGISFMNWFQQKPGKAPK
LLIYAASNQGSGVPSRFSGSGSGTDFTLTISSLQPEDFATYFCQQSKEV
PWTFGGGTKLEIK;
and
(SEQ ID NO: 53)
DIQMTQSPSSLSASVGDRVTITCRASESVDNYGISFMNWFQQKPGKPPK
LLIYAASNQGSGVPSRFSGSGSGTDFTLTISSLQPEDFATYYCQQSKEV
PWTFGGGTKLEIK.

In another aspect, the disclosure provides an anti-mTTR antibody Ab2 comprising a heavy chain variable domain (VH) comprising the sequence

(SEQ ID NO: 54)
EVKLEQSGGGLVQPGGSMKLSCVASGFTFSNYWMNWVRQSPEKGLEWVA
QIRLKSDNYATHYAESVKGRFTISRDDSKSSVYLQMNNLRAEDTGIYYC
TGVFTYWGQGTLVTVSA.

In another aspect, the disclosure provides an anti-mTTR antibody comprising a heavy chain variable domain (VH) comprising a sequence selected from

(SEQ ID NO: 55)
EVQLVESGGGLVKPGGSLRLSCAASGFTFSNYWMNWVRQAPGKGLEWVG
QIRLKSDNYATHYAAPVKGRFTISRDDSKNTLYLQMNSLKTEDTAVYYC
TTVFTYWGQGTLVTVSS,
(SEQ ID NO: 56)
EVQLVESGGGLVKPGGSLRLSCAASGFTFSNYWMNWVRQAPGKGLEWVG
QIRLKSDNYATHYAAPVKGRFTISRDDSKNTLYLQMNSLKTEDTAVYYC
TGVFTYWGQGTLVTVSS,
(SEQ ID NO: 57)
EVQLVESGGGLVKPGGSLRLSCAASGFTFSNYWMNWVRQAPGKGLEWVA
QIRLKSDNYATHYAAPVKGRFTISRDDSKNTVYLQMNSLKTEDTAVYYC
TGVFTYWGQGTLVTVSS,
(SEQ ID NO: 58)
EVQLLESGGGLVQPGGSLRLSCAASGFTFSNYWMNWVRQAPGKGLEWVS
QIRLKSDNYATHYADSVKGRFTISRDNSKNTLYLQMNSLRAEDTAVYYC
AKVFTYWGQGTLVTVSS,
(SEQ ID NO: 59)
EVQLLESGGGLVQPGGSLRLSCAASGFTFSNYWMNWVRQAPGKGLEWVS
QIRLKSDNYATHYADSVKGRFTISRDNSKNTLYLQMNSLRAEDTAVYYC
TGVFTYWGQGTLVTVSS,
and
(SEQ ID NO: 60)
EVQLLESGGGLVQPGGSLRLSCAASGFTFSNYWMNWVRQAPGKGLEWVA
QIRLKSDNYATHYADSVKGRFTISRDDSKNTVYLQMNSLRAEDTAVYYC
TGVFTYWGQGTLVTVSS.

In another aspect, the disclosure provides an anti-mTTR antibody Ab2 comprising a light chain variable domain (VL) comprising the sequence

(SEQ ID NO: 61)
NIVLTQSPASLAVSLGQRATISCRTSESVDSYGNSFMHWYQQKPGQPPK
LLIYLASNLESGVPARFSGSGSRTDFTLTIDPVEADDAATYYCQQNNED
PMYTFGGGTKLEIK.

In another aspect, the disclosure provides a humanized anti-mTTR antibody comprising a light chain variable domain (VL) comprising the sequence selected from

(SEQ ID NO: 62)
DIVMTQSPDSLAVSLGERATINCRTSESVDSYGNSFMHWYQQKPGQPPK
LLIYLASNLESGVPDRFSGSGSGTDFTLTISSLQAEDVAVYYCQQNNED
PMYTFGGGTKVEIK,
(SEQ ID NO: 63)
DIVMTQSPDSLAVSLGERATINCRTSESVDSYGNSFMHWYQQKPGQPPK
LLIYLASNLESGVPDRFSGSGSRTDFTLTISSLQAEDVAVYYCQQNNED
PMYTFGGGTKVEIK,
and
(SEQ ID NO: 64)
DIVLTQSPDSLAVSLGERATINCRTSESVDSYGNSFMHWYQQKPGQPPK
LLIYLASNLESGVPDRFSGSGSRTDFTLTISSLQAEDVAVYYCQQNNED
PMYTFGGGTKVEIK.

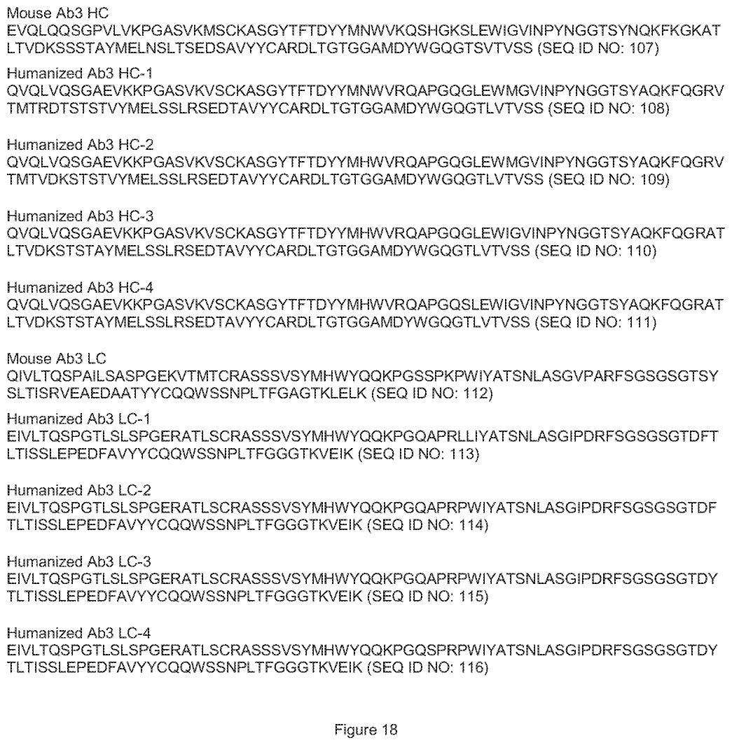

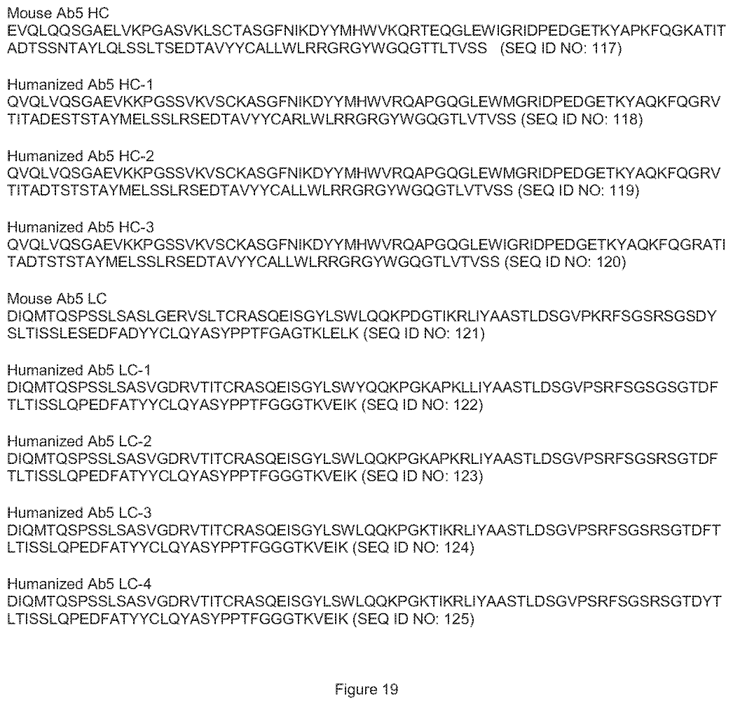

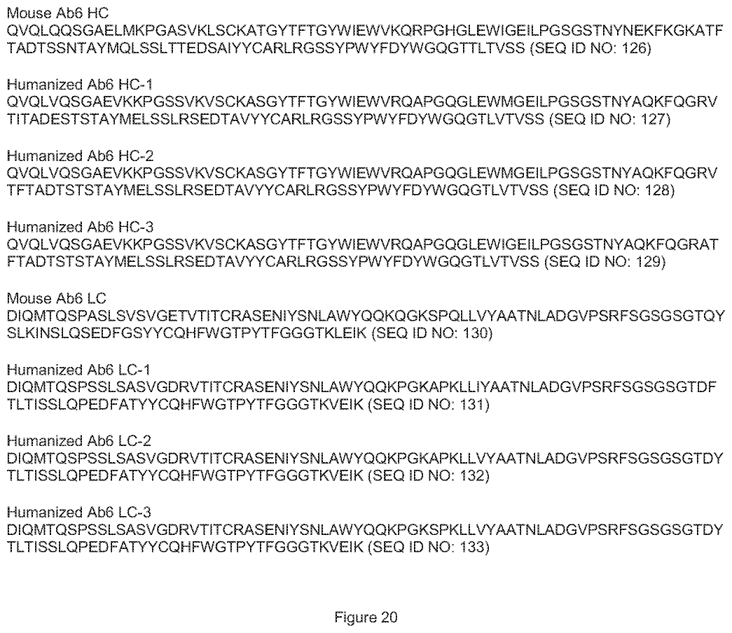

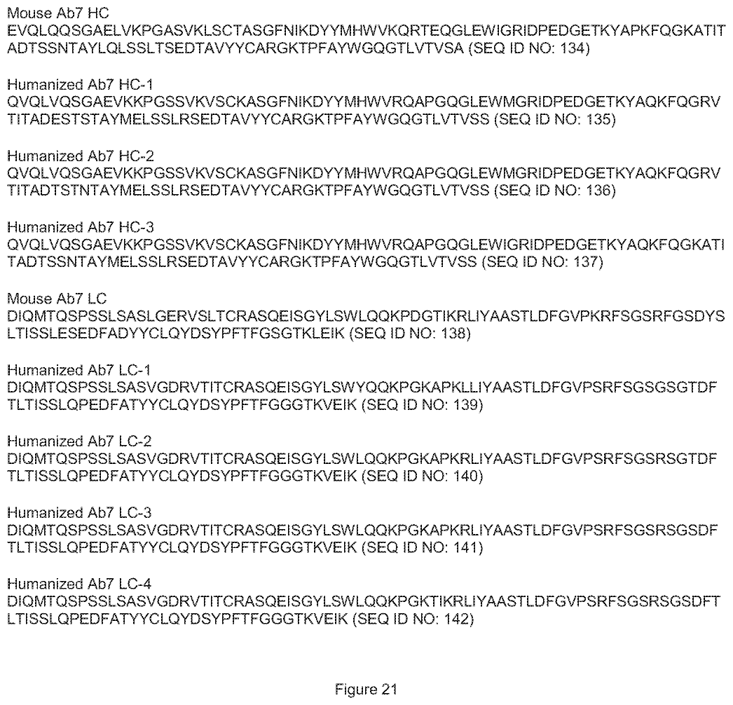

In another aspect, the disclosure provides a humanized anti-mTTR antibody comprising a light chain variable domain (VL) comprising the sequence selected from those disclosed in FIGS. 18-21. In another aspect, the disclosure provides a humanized anti-mTTR antibody comprising a heavy chain variable domain (VH) comprising the sequence selected from those disclosed in FIGS. 18-21.

In any of the aspects provided herein, an anti-TTR antibody is humanized. In any of the aspects provided herein, an anti-TTR antibody is a chimera. In one aspect, an anti-TTR antibody further comprises an acceptor human framework, e.g. a human immunoglobulin framework or a human consensus framework.

In another aspect, an anti-TTR antibody comprises a VL domain comprising at least 85%, 86%, 87%, 88%, 89%, 90%, 91%, 92%, 93%, 94%, 95%, 96%, 97%, 98% or 99% sequence identity to any one of SEQ ID NO: 51-53. In one aspect, the VL domain comprises at least 85% sequence identity with SEQ ID NO: 51-53. In one aspect, the VL domain comprises at least 95% sequence identity with SEQ ID NO: 51-53. In another aspect, the VL domain comprises at least 98% sequence identity with any one of SEQ ID NO: 51-53.

In another aspect, an anti-TTR antibody comprises a VL domain comprising at least 85%, 86%, 87%, 88%, 89%, 90%, 91%, 92%, 93%, 94%, 95%, 96%, 97%, 98% or 99% sequence identity to any one of SEQ ID NO: 62-64. In one aspect, the VL domain comprises at least 85% sequence identity with SEQ ID NO: 62-64. In one aspect, the VL domain comprises at least 95% sequence identity with SEQ ID NO: 62-64. In another aspect, the VL domain comprises at least 98% sequence identity with any one of SEQ ID NO: 62-64.

In another aspect, an anti-TTR antibody comprises a VH domain comprising at least 85%, 86%, 87%, 88%, 89%, 90%, 91%, 92%, 93%, 94%, 95%, 96%, 97%, 98% or 99% sequence identity to any one of SEQ ID NO: 46-49. In one aspect, the VH domain comprises at least 85% sequence identity with SEQ ID NO: 46-49. In one aspect, the VH domain comprises at least 95% sequence identity with SEQ ID NO: 46-49. In another aspect, the VH domain comprises at least 98% sequence identity with any one of SEQ ID NO: 46-49.

In another aspect, an anti-TTR antibody comprises a VH domain comprising at least 85%, 86%, 87%, 88%, 89%, 90%, 91%, 92%, 93%, 94%, 95%, 96%, 97%, 98% or 99% sequence identity to any one of SEQ ID NO: 55-60. In one aspect, the VH domain comprises at least 85% sequence identity with SEQ ID NO: 55-60. In one aspect, the VH domain comprises at least 95% sequence identity with SEQ ID NO: 55-60. In another aspect, the VH domain comprises at least 98% sequence identity with any one of SEQ ID NO: 55-60.

In another aspect, an anti-TTR antibody comprises one or more of the CDR sequences of a VH selected from SEQ ID NOs: 46-49. In another embodiment, an anti-TTR antibody comprises one or more of the CDR sequences of a VL selected from SEQ ID NOS: 51-53. In another embodiment, an anti-TTR antibody comprises the CDR sequences of a VH selected from SEQ ID NO: 46-49 and the CDR sequences of a VL selected from SEQ ID NOs: 51-53.

In one aspect, an anti-TTR antibody comprises one or more of the heavy chain CDR amino acid sequences of a VH selected from SEQ ID NOs: 46-49 and a framework of at least 85%, 86%, 87%, 88%, 89%, 90%, 91%, 92%, 93%, 94%, 95%, 96%, 97%, 98%, 99% sequence identity to the framework amino acid sequence of a VH selected from SEQ ID NOs: 46-49. In one aspect, the anti-TTR antibody comprises the three heavy chain CDR amino acid sequences of a VH selected from SEQ ID NOs: 46-49 and a framework of at least 85%, 86%, 87%, 88%, 89%, 90%, 91%, 92%, 93%, 94%, 95%, 96%, 97%, 98%, 99% sequence identity to the framework amino acid sequence of a VH selected from SEQ ID NOs: 46-49. In one aspect, the anti-TTR antibody comprises the three heavy chain CDR amino acid sequences of a VH selected from SEQ ID NOs: 46-49 and a framework of at least 85% sequence identity to the framework amino acid sequence of a VH selected from SEQ ID NOs: 46-49. In one aspect, the anti-TTR antibody comprises the three heavy chain CDR amino acid sequences of a VH selected from SEQ ID NOs: 46-49 and a framework of at least 85% or at least 95% sequence identity to the framework amino acid sequence of a VH selected from SEQ ID NOs: 46-49. In another aspect, the anti-TTR antibody comprises the three heavy chain CDR amino acid sequences of a VH selected from SEQ ID NOs: 46-49 and a framework of at least of at least 98% sequence identity to the framework amino acid sequence of a VH selected from SEQ ID NOs: 46-49.

In one aspect, an anti-TTR antibody comprises one or more of the light chain CDR amino acid sequences of a VL selected from SEQ ID NOs: 51-53 and a framework of at least 85%, 86%, 87%, 88%, 89%, 90%, 91%, 92%, 93%, 94%, 95%, 96%, 97%, 98%, 99% sequence identity to the framework amino acid sequence of a VL selected from SEQ ID NOs: 51-53. In one aspect, the anti-TTR antibody comprises the three light chain CDR amino acid sequences of a VL selected from SEQ ID NOs: 51-53 and a framework of at least 85%, 86%, 87%, 88%, 89%, 90%, 91%, 92%, 93%, 94%, 95%, 96%, 97%, 98%, 99% sequence identity to the framework amino acid sequence of a VL selected from SEQ ID NOs: 51-53. In one aspect, the anti-TTR antibody comprises the three light chain CDR amino acid sequences of a VL selected from SEQ ID NOs: 51-53 and a framework of at least 85% sequence identity to the framework amino acid sequence of a VL selected from SEQ ID NOs: 51-53. In one aspect, the anti-TTR antibody comprises the three light chain CDR amino acid sequences of a VL selected from SEQ ID NOs: 51-53 and a framework of at least 85% or at least 95% sequence identity to the framework amino acid sequence of a VL selected from SEQ ID NOs: 51-53. In another aspect, the anti-TTR antibody comprises the three light chain CDR amino acid sequences of a VL selected from SEQ ID NOs: 51-53 and a framework of at least particularly of at least 98% sequence identity to the framework amino acid sequence of a VL selected from SEQ ID NOs: 51-53.

In one aspect, the anti-TTR antibody comprises (a) CDR-H1 comprising the amino acid sequence of SEQ ID NOs: 2-8; (b) CDR-H2 comprising the amino acid sequence of SEQ ID NOs: 9-15; (c) CDR-H3 comprising the amino acid sequence of SEQ ID NOs: 16-22; (d) CDR-L1 comprising the amino acid sequence of SEQ ID NOs: 23-29; (e) CDR-L2 comprising the amino acid sequence of SEQ ID NOs: 30-36; and (f) CDR-L3 comprising the amino acid sequence of SEQ ID NOs: 37-43, and a VH domain having at least 90%, 91%, 92%, 93%, 94%, 95%, 96%, 97%, 98%, 99%, or 100% sequence identity to an amino acid sequence selected from SEQ ID NOs: 46-49, and a VL domain having at least 90%, 91%, 92%, 93%, 94%, 95%, 96%, 97%, 98%, 99%, or 100% sequence identity to an amino acid sequence selected from SEQ ID NOs: 51-53. In one aspect, the VH domain has at least 85% sequence identity to an amino acid sequence selected from SEQ ID NOs: 46-49. In one aspect, the VH domain has at least 95% sequence identity to an amino acid sequence selected from SEQ ID NOs: 46-49. In one aspect, the VL domain has at least 85% sequence identity to an amino acid sequence selected from SEQ ID NOs: 51-53. In one aspect, the VL domain has at least 95% sequence identity to an amino acid sequence selected from SEQ ID NOs: 51-53.

In one aspect, the anti-TTR antibody comprises (a) CDR-H1 comprising the amino acid sequence of SEQ ID NOs: 2-8; (b) CDR-H2 comprising the amino acid sequence of SEQ ID NOs: 9-15; (c) CDR-H3 comprising the amino acid sequence of SEQ ID NOs: 16-22; (d) CDR-L1 comprising the amino acid sequence of SEQ ID NOs: 23-29; (e) CDR-L2 comprising the amino acid sequence of SEQ ID NOs: 30-36; and (f) CDR-L3 comprising the amino acid sequence of SEQ ID NOs: 37-43, and a VH domain having at least 90%, 91%, 92%, 93%, 94%, 95%, 96%, 97%, 98%, 99%, or 100% sequence identity to an amino acid sequence selected from SEQ ID NOs: 46-49, and a VL domain having at least 90%, 91%, 92%, 93%, 94%, 95%, 96%, 97%, 98%, 99%, or 100% sequence identity to an amino acid sequence selected from SEQ ID NOs: 51-53; wherein the antibody specifically binds to TTR monomer. In one aspect, the VH domain has at least 85% sequence identity to an amino acid sequence selected from SEQ ID NOs: 46-49. In one aspect, the VH domain has at least 95% sequence identity to an amino acid sequence selected from SEQ ID NOs: 46-49. In one aspect, the VL domain has at least 85% sequence identity to an amino acid sequence selected from SEQ ID NOs: 51-53. In one aspect, the VL domain has at least 95% sequence identity to an amino acid sequence selected from SEQ ID NOs: 51-53.

In another aspect, an anti-TTR antibody is provided, wherein the antibody comprises a VH sequence as in any of the aspects provided above, and a VL sequence as in any of the aspects provided above. In one aspect, the antibody comprises the VH and VL sequences in SEQ ID NO: 46 and SEQ ID NO: 51, respectively, including post-translational modifications of those sequences. In one aspect, the antibody comprises the VH and VL sequences in SEQ ID NO: 49 and SEQ ID NO: 53, respectively, including post-translational modifications of those sequences.

In another aspect, an anti-TTR antibody comprises one or more of the CDR sequences of a VH selected from SEQ ID NOs: 55-60. In another embodiment, an anti-TTR antibody comprises one or more of the CDR sequences of a VL selected from SEQ ID NOS: 62-64. In another embodiment, an anti-TTR antibody comprises the CDR sequences of a VH selected from SEQ ID NO: 55-60 and the CDR sequences of a VL selected from SEQ ID NOs: 62-64.

In one aspect, an anti-TTR antibody comprises one or more of the heavy chain CDR amino acid sequences of a VH selected from SEQ ID NOs: 55-60 and a framework of at least 85%, 86%, 87%, 88%, 89%, 90%, 91%, 92%, 93%, 94%, 95%, 96%, 97%, 98%, 99% sequence identity to the framework amino acid sequence of a VH selected from SEQ ID NOs: 55-60. In one aspect, the anti-TTR antibody comprises the three heavy chain CDR amino acid sequences of a VH selected from SEQ ID NOs: 55-60 and a framework of at least 85%, 86%, 87%, 88%, 89%, 90%, 91%, 92%, 93%, 94%, 95%, 96%, 97%, 98%, 99% sequence identity to the framework amino acid sequence of a VH selected from SEQ ID NOs: 55-60. In one aspect, the anti-TTR antibody comprises the three heavy chain CDR amino acid sequences of a VH selected from SEQ ID NOs: 55-60 and a framework of at least 85% or at least 95% sequence identity to the framework amino acid sequence of a VH selected from SEQ ID NOs: 55-60. In another aspect, the anti-TTR antibody comprises the three heavy chain CDR amino acid sequences of a VH selected from SEQ ID NOs: 55-60 and a framework of at least of at least 98% sequence identity to the framework amino acid sequence of a VH selected from SEQ ID NOs: 55-60.

In one aspect, an anti-TTR antibody comprises one or more of the light chain CDR amino acid sequences of a VL selected from SEQ ID NOs: 62-64 and a framework of at least 85%, 86%, 87%, 88%, 89%, 90%, 91%, 92%, 93%, 94%, 95%, 96%, 97%, 98%, 99% sequence identity to the framework amino acid sequence of a VL selected from SEQ ID NOs: 62-64. In one aspect, the anti-TTR antibody comprises the three light chain CDR amino acid sequences of a VL selected from SEQ ID NOs: 62-64 and a framework of at least 85%, 86%, 87%, 88%, 89%, 90%, 91%, 92%, 93%, 94%, 95%, 96%, 97%, 98%, 99% sequence identity to the framework amino acid sequence of a VL selected from SEQ ID NOs: 62-64. In one aspect, the anti-TTR antibody comprises the three light chain CDR amino acid sequences of a VL selected from SEQ ID NOs: 62-64 and a framework of at least 85% or at least 95% sequence identity to the framework amino acid sequence of a VL selected from SEQ ID NOs: 62-64. In another aspect, the anti-TTR antibody comprises the three light chain CDR amino acid sequences of a VL selected from SEQ ID NOs: 62-64 and a framework of at least particularly of at least 98% sequence identity to the framework amino acid sequence of a VL selected from SEQ ID NOs: 62-64.

In one aspect, the anti-TTR antibody comprises (a) CDR-H1 comprising the amino acid sequence of SEQ ID NOs: 2-8; (b) CDR-H2 comprising the amino acid sequence of SEQ ID NOs: 9-15; (c) CDR-H3 comprising the amino acid sequence of SEQ ID NOs: 16-22; (d) CDR-L1 comprising the amino acid sequence of SEQ ID NOs: 23-29; (e) CDR-L2 comprising the amino acid sequence of SEQ ID NOs: 30-36; and (f) CDR-L3 comprising the amino acid sequence of SEQ ID NOs: 37-43, and a VH domain having at least 90%, 91%, 92%, 93%, 94%, 95%, 96%, 97%, 98%, 99%, or 100% sequence identity to an amino acid sequence selected from SEQ ID NOs: 46-49, and a VL domain having at least 90%, 91%, 92%, 93%, 94%, 95%, 96%, 97%, 98%, 99%, or 100% sequence identity to an amino acid sequence selected from SEQ ID NOs: 62-64. In one aspect, the VH domain has at least 85% or at least 95% sequence identity to an amino acid sequence selected from SEQ ID NOs: 55-60. In one aspect, the VL domain has at least 85% or at least 95% sequence identity to an amino acid sequence selected from SEQ ID NOs: 62-64.

In one aspect, the anti-TTR antibody comprises (a) CDR-H1 comprising the amino acid sequence of SEQ ID NOs: 2-8; (b) CDR-H2 comprising the amino acid sequence of SEQ ID NOs: 9-15; (c) CDR-H3 comprising the amino acid sequence of SEQ ID NOs: 16-22; (d) CDR-L1 comprising the amino acid sequence of SEQ ID NOs: 23-29; (e) CDR-L2 comprising the amino acid sequence of SEQ ID NOs: 30-36; and (f) CDR-L3 comprising the amino acid sequence of SEQ ID NOs: 37-43, and a VH domain having at least 90%, 91%, 92%, 93%, 94%, 95%, 96%, 97%, 98%, 99%, or 100% sequence identity to an amino acid sequence selected from SEQ ID NOs: 55-60, and a VL domain having at least 90%, 91%, 92%, 93%, 94%, 95%, 96%, 97%, 98%, 99%, or 100% sequence identity to an amino acid sequence selected from SEQ ID NOs: 62-64; wherein the antibody specifically binds to TTR monomer. In one aspect, the VH domain has at least 85% or at least 95% sequence identity to an amino acid sequence selected from SEQ ID NOs: 55-60. In one aspect, the VL domain has at least 85% or at least 95% sequence identity to an amino acid sequence selected from SEQ ID NOs: 62-64.

In another aspect, an anti-TTR antibody is provided, wherein the antibody comprises a VH sequence as in any of the aspects provided above, and a VL sequence as in any of the aspects provided above. In one aspect, the antibody comprises the VH and VL sequences in SEQ ID NO: 60 and SEQ ID NO: 64, respectively, including post-translational modifications of those sequences. In one aspect, the antibody comprises the VH and VL sequences in SEQ ID NO: 55 and SEQ ID NO: 62, respectively, including post-translational modifications of those sequences.

In a further aspect, the disclosure provides an antibody that binds to the same epitope as an anti-TTR antibody provided herein. For example, in certain aspects, an antibody is provided that binds to the same epitope as an anti-TTR antibody comprising a VH sequence of any of SEQ ID NO: 45-49 and a VL sequence of any of SEQ ID NO: 50-53.

In some embodiments, the anti-TTR antibody does not bind to a peptide selected from GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69). LSPYSY (SEQ ID NO: 70). SYSTTAVV (SEQ ID NO: 71), YTIAALLSPYSYSTTAVV (SEQ ID NO: 72); LGISPMHEHAE (SEQ ID NO: 73); and RRYTIAAMLSPYS (SEQ ID NO: 74). In some embodiments, the anti-TTR antibody does not bind to GPTGTGESKCPL (SEQ ID NO: 65). In some embodiments, the anti-TTR antibody does not bind to MVKVLDAVRGSPA (SEQ ID NO: 66). In some embodiments, the anti-TTR antibody does not bind to PFHEHAEVFTA (SEQ ID NO: 67). In some embodiments, the anti-TTR antibody does not bind to EHAEVVFTA (SEQ ID NO: 68). In some embodiments, the anti-TTR antibody does not bind to YTIAALLS (SEQ ID NO: 69). In some embodiments, the anti-TTR antibody does not bind to LSPYSY (SEQ ID NO: 70). In some embodiments, the anti-TTR antibody does not bind to SYSTTAVV (SEQ ID NO: 71). In some embodiments, the anti-TTR antibody does not bind to YTIAALLSPYSYSTTAVV (SEQ ID NO: 72). In some embodiments, the anti-TTR antibody does not bind to LGISPMHEHAE (SEQ ID NO: 73). In some embodiments, the anti-TTR antibody does not bind to RRYTIAAMLSPYS (SEQ ID NO: 74).

In a further aspect, the disclosure provides an antibody that binds to a conformational epitope.

In a further aspect, the disclosure provides an antibody that competes for binding to TTR monomer with an anti-TTR antibody provided herein.

In a further aspect, the disclosure provides an antibody that competes for binding to TTR monomer with an anti-TTR antibody provided herein in an indirect ELISA.

In a further aspect of the disclosure, an anti-TTR antibody according to any of the above aspects is a monoclonal antibody, including a chimeric, humanized or human antibody. In one aspect, an anti-TTR antibody is an antibody fragment, e.g., a Fv, Fab, Fab′, scFv, diabody, or F(ab′)2 fragment. In another aspect, the antibody is a full-length antibody. e.g., an intact IgG4 antibody or other antibody class or isotype as defined herein.

In a further aspect, an anti-TTR antibody according to any of the above aspects may incorporate any of the features, singly or in combination, as described below.

This disclosure also encompasses specific humanized antibody variable domains, and isolated polypeptides having homology with the following sequences:
i) SEQ ID NO: 53, which is the light chain variable domain of a humanized version of Ab1;
ii) SEQ ID NO: 49, which is the heavy chain variable domain of a humanized version of Ab1;
iii) SEQ ID NO: 64 which is the light chain variable domain of a humanized version of Ab2; and
iv) SEQ ID NO: 60, which is the heavy chain variable domain of a humanized version of Ab2.

In an alternative method, KD is measured by bio-layer interferometry.

In another aspect, an anti-TTR antibody is provided, wherein the antibody has an affinity of about <10 nM (KD) to TTR monomer as determined by bio-layer interferometry (BLI) or surface plasmon resonance (SPR) assays.

In another aspect, an anti-TTR antibody is provided, wherein the antibody has at least 5:1 specificity (by KD ratio) to TTR monomer over TTR tetramer (WT or stabilized version) measured by bio-layer interferometry (BLI) or surface plasmon resonance (SPR) assays.

In another aspect, an anti-TTR antibody is provided, wherein the antibody is a mouse IgG2a.

In another aspect, an anti-TTR antibody is provided, wherein the antibody is a mouse IgG2a mAb with TTR monomer binding in 20% human serum.

In another aspect, an anti-TTR antibody is provided, wherein the antibody is an IgG4.

In another aspect, an anti-TTR monomer antibody is provided, wherein the antibody is used to treat TTR amyloid fibril formation or amyloidogenesis. TTR amyloid fibril formation or amyloidogenesis is associated with a number of diseases, which present a significant burden on human health. For example, wild-type TTR amyloidogenesis is associated with senile systemic amyloidosis (SSA), a cardiomyopathy affecting up to 25% of the population above the age of 80. Another example of a TTR-related amyloid disease is the autosomal dominantly inherited disorder familial amyloid polyneuropathy (FAP).

In another aspect, an anti-TTR monomer antibody is provided, wherein the antibody is used to treat familial amyloid cardiomyopathy. Familial amyloid cardiomyopathy (FAC), another inherited TTR-aggregation-associated disease, can lead to congestive heart failure and death. In another aspect, an anti-TTR monomer antibody is provided, wherein the antibody is used to treat hereditary amyloid diseases associated with TTR point mutations.

In another aspect, the anti-TTR monomer antibody is used to treat ATTR-PN and ATTR-CM, including hATTR or wtATTR.

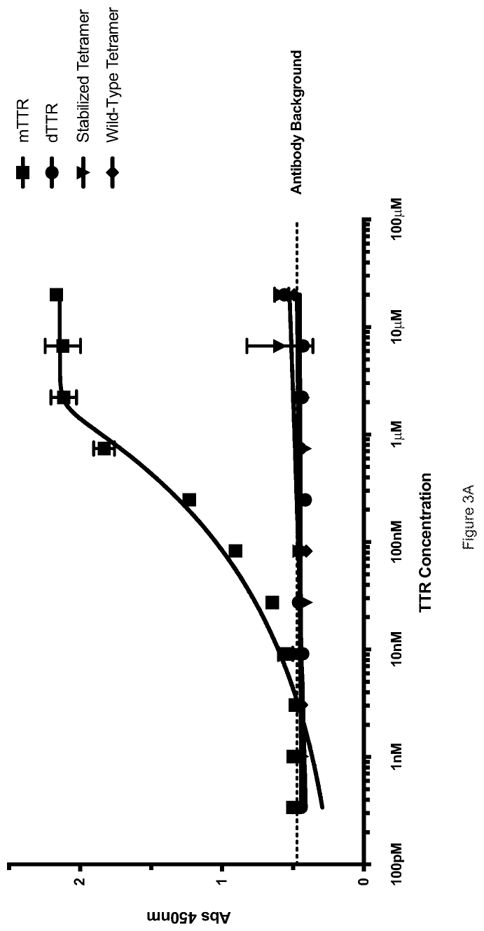

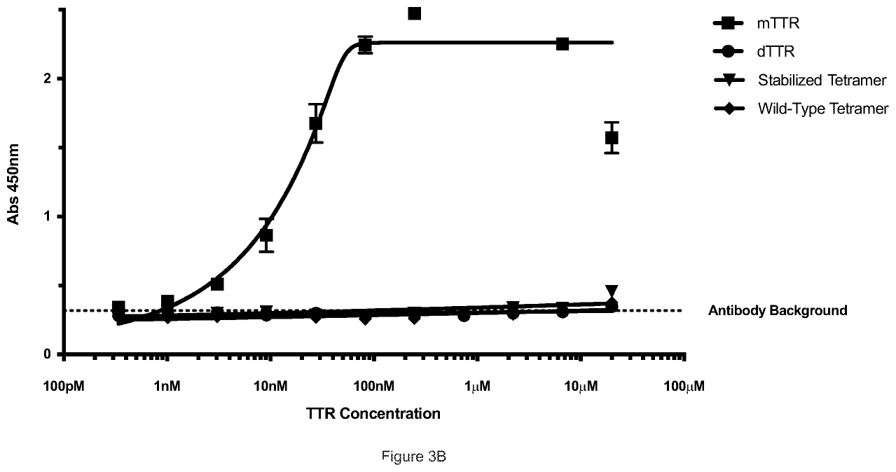

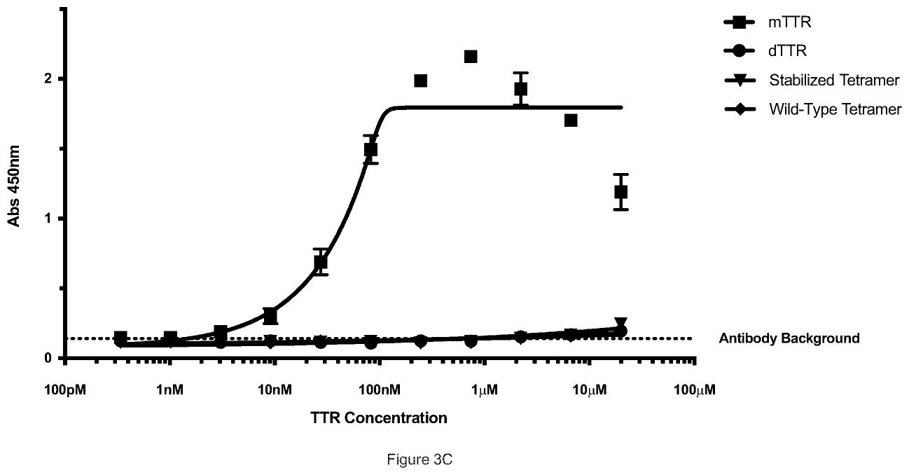

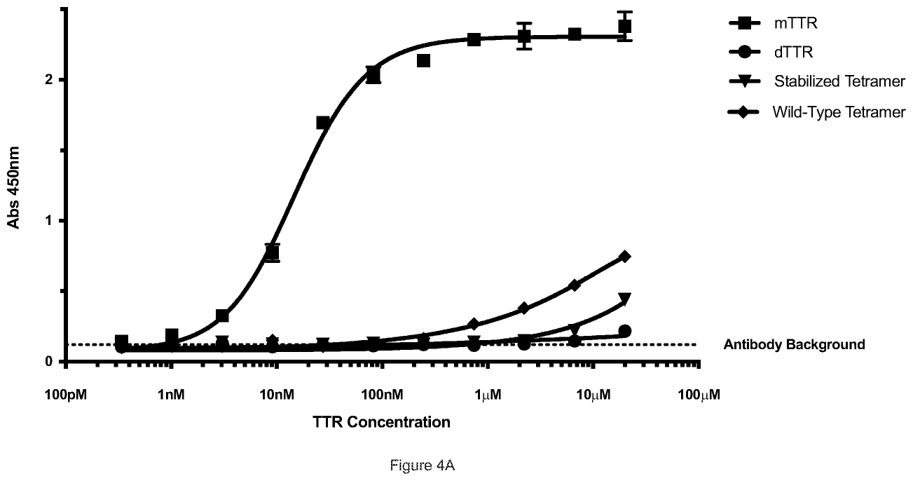

In another embodiment, the present disclosure refers to an antibody or antibody fragment that cross-competes with an antibody described in FIG. 3.

In one embodiment the present disclosure refers to an antibody or antibody fragment, wherein said antibody or antibody fragment cross-competes with an antibody or antibody fragment comprising 6 CDRs defined by any of the Kabat, Chothia, IMGT, or combined Kabat/Chothia method of one or more of the antibodies in FIG. 3.

In another embodiment, the present disclosure refers to an antibody or antibody fragment that binds to (e.g., by binding and/or stabilizing) the same epitope as one of the antibodies in FIG. 3.

In a further embodiment said antibody or antigen-binding fragment thereof binds to (e.g., by binding and/or stabilizing) an epitope overlapping the epitope of an antibody or antibody fragment comprising 6 CDRs defined by any one of the Kabat, Chothia, IMGT, or combined Kabat/Chothia methods of any one of the antibodies in FIG. 3.

### Antibody Fragments

In certain aspects, an antibody provided herein is an antibody fragment.

In one aspect, the antibody fragment is a Fab, Fab′, Fab′-SH, or F(ab′)2 fragment, in particular a Fab fragment. Papain digestion of intact antibodies produces two identical antigen-binding fragments, called “Fab” fragments containing each the heavy- and light-chain variable domains (VH and VL, respectively) and also the constant domain of the light chain (CL) and the first constant domain of the heavy chain (CH1). The term “Fab fragment” thus refers to an antibody fragment comprising a light chain comprising a VL domain and a CL domain, and a heavy chain fragment comprising a VH domain and a CH1 domain. “Fab′ fragments” differ from Fab fragments by the addition of residues at the carboxy terminus of the CH1 domain including one or more cysteines from the antibody hinge region. Fab′-SH are Fab′ fragments in which the cysteine residue(s) of the constant domains bear a free thiol group. Pepsin treatment yields an F(ab′)2 fragment that has two antigen-binding sites (two Fab fragments) and a part of the Fc region. For discussion of Fab and F(ab′)2 fragments comprising salvage receptor binding epitope residues and having increased in vivo half-life, see U.S. Pat. No. 5,869,046.

In another aspect, the antibody fragment is a diabody, a triabody or a tetrabody. “Diabodies” are antibody fragments with two antigen-binding sites. See, for example, EP 404,097; WO 1993/01161; Hudson et al., Nat. Med. 9:129-134 (2003); and Hollinger et al., Proc. Natl. Acad. Sci. USA 90:6444-6448 (1993). Triabodies and tetrabodies are also described in Hudson et al., Nat. Med. 9:129-134 (2003).

In a further aspect, the antibody fragment is a single chain Fab fragment.

A “single chain Fab fragment” or “scFab” is a polypeptide consisting of an antibody heavy chain variable domain (VH), an antibody heavy chain constant domain 1 (CHI), an antibody light chain variable domain (VL), an antibody light chain constant domain (CL) and a linker, wherein said antibody domains and said linker have one of the following orders in N-terminal to C-terminal direction: a) VH-CHI-linker-VL-CL, b) VL-CL-linker-VH-CHI, c) VH-CL-linker-VL-CH1 or d) VL-CHI-linker-VH-CL. In particular, said linker is a polypeptide of at least 30 amino acids, preferably between 32 and 50 amino acids. Said single chain Fab fragments are stabilized via the natural disulfide bond between the CL domain and the CHI domain. In addition, these single chain Fab fragments might be further stabilized by generation of interchain disulfide bonds via insertion of cysteine residues (e.g., position 44 in the variable heavy chain and position 100 in the variable light chain according to Kabat numbering).

In another aspect, the antibody fragment is single-chain variable fragment (scFv). A “single-chain variable fragment” or “scFy” is a fusion protein of the variable domains of the heavy (VH) and light chains (VL) of an antibody, connected by a linker. In particular, the linker is a short polypeptide of 10 to 25 amino acids and is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. For a review of scFv fragments, see, e.g., Pliickthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458.

In another aspect, the antibody fragment is a single-domain antibody. “Single-domain antibodies” are antibody fragments comprising all or a portion of the heavy chain variable domain or all or a portion of the light chain variable domain of an antibody. In certain aspects, a single-domain antibody is a human single-domain antibody (Domantis, Inc., Waltham, MA; see, e.g., U.S. Pat. No. 6,248,516 BI).

Antibody fragments can be made by various techniques, including but not limited to proteolytic digestion of an intact antibody as well as recombinant production by recombinant host cells (e.g., E. coli), as described herein.

### Chimeric and Humanized Antibodies

In certain aspects, an antibody provided herein is a chimeric antibody. Certain chimeric antibodies are described, e.g., in U.S. Pat. No. 4,816,567; and Morrison et al., Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain aspects, a chimeric antibody is a humanized antibody.

Typically, a non-human antibody is humanized to reduce immunogenicity to humans, while retaining the specificity and affinity of the parental non-human antibody. Generally, a humanized antibody comprises one or more variable domains in which the CDRs (or portions thereof) are derived from a non-human antibody, and FRs (or portions thereof) are derived from human antibody sequences. A humanized antibody optionally will also comprise at least a portion of a human constant region. In some aspects, some FR residues in a humanized antibody are substituted with corresponding residues from a non-human antibody (e.g., the antibody from which the CDR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat 1 Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting): Padlan, Mol. Immunol. 28:489-498 (1991) (describing “resurfacing”); DalFAcqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993); human mature (somatically mutated) framework regions of human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

### Human Antibodies

In certain aspects, an antibody provided herein is a human antibody. Human antibodies can be produced using various techniques known in the art. Human antibodies are described generally in van Dijk and van de Winkel, Curr. Opin. Pharmacol. 5:368-74 (2001) and Lonberg, Curr. Opin. Immunol. 20:450-459 (2008).

Human antibodies may be prepared by administering an immunogen to a transgenic animal that has been modified to produce intact human antibodies or intact antibodies with human variable regions in response to antigenic challenge. Such animals typically contain all or a portion of the human immunoglobulin loci, which replace the endogenous immunoglobulin loci, or which are present extra-chromosomally or integrated randomly into the animal's chromosomes. In such transgenic mice, the endogenous immunoglobulin loci have generally been inactivated. For review of methods for obtaining human antibodies from transgenic animals, see Lonberg, Nat. Biotech. 23:1117-1125 (2005). See also, e.g., U.S. Pat. Nos. 6,075,181 and 6,150,584 describing XENOMOUSE™ technology; Ei.S. U.S. Pat. No. 5,770,429 describing HUMAB® technology; El.S. U.S. Pat. No. 7,041,870 describing K-M MOUSE® technology, and U.S. Patent Application Publication No. US 2007/0061900, describing VELOCIMOUSE® technology). Human variable regions from intact antibodies generated by such animals may be further modified, e.g., by combining with a different human constant region.

Human antibodies can also be made by hybridoma-based methods.

Human myeloma and mouse-human heteromyeloma cell lines for the production of human monoclonal antibodies have been described. (See, e.g., Kozbor J. Immunol., 133:3001 (1984); Brodeur et al., Monoclonal Antibody Production Techniques and Applications, pp. 51-63 (Marcel Dekker, Inc., New York, 1987); and Boerner et al., J. Immunol., 147:86 (1991).) Human antibodies generated via human B-cell hybridoma technology are also described in Li et al., Proc. Natl. Acad. Sci. USA, 103:3557-3562 (2006). Additional methods include those described, for example, in U.S. Pat. No. 7,189,826 (describing production of monoclonal human IgM antibodies from hybridoma cell lines) and Ni, Xiandai Mianyixue, 26 (4): 265-268 (2006) (describing human-human hybridomas). Human hybridoma technology (Trioma technology) is also described in Vollmers and Brandlein, Histology and Histopathology, 20 (3): 927-937 (2005) and Vollmers and Brandlein, Methods and Findings in Experimental and Clinical Pharmacology, 27 (3): 185-91 (2005).

Human antibodies may also be generated by isolating variable domain sequences selected from human-derived phage display libraries. Such variable domain sequences may then be combined with a desired human constant domain. Techniques for selecting human antibodies from antibody libraries are described below.

### B-Cell Sorting

Human antibodies may be generated by single cell dissection of spleen cells from an animal such as a mouse using technology like the proprietary Beacon platform offered by Berkeley Lights or by AbCellera.

### Library-Derived Antibodies

In certain aspects, an antibody provided herein is derived from a library. Antibodies of the disclosure, in various aspects, are isolated by screening combinatorial libraries for antibodies with the desired activity of activities. Methods for screening combinatorial libraries are reviewed, e.g., in Lemer et al, in Nature Reviews 16:498-508 (2016). For example, a variety of methods are known in the art for generating phage display libraries and screening such libraries for antibodies possessing the desired binding characteristics. Such methods are reviewed, e.g., in Frenzel et al, in mAbs 8:1177-1194 (2016); Bazan et al, in Human Vaccines and Immunotherapeutics 8:1817-1828 (2012) and Zhao et al, in Critical Reviews in Biotechnology 36:276-289 (2016) as well as in Hoogenboom et al, in Methods in Molecular Biology 178:1-37 (O'Brien et al., ed., Human Press, Totowa, NJ, 2001) and in Marks and Bradbury in Methods in Molecular Biology 248:161-175 (Lo, ed., Human Press, Totowa, NJ, 2003).

In certain phage display methods, repertoires of VH and VL genes are separately cloned by polymerase chain reaction (PCR) and recombined randomly in phage libraries, which can then be screened for antigen-binding phage as described in Winter et al, in Annual Review of Immunology 12:433-455 (1994). Phage typically display antibody fragments, either as single-chain Fv (scFv) fragments or as Fab fragments. Libraries from immunized sources provide high-affinity antibodies to the immunogen without the requirement of constructing hybridomas. Alternatively, the naive repertoire can be cloned (e.g., from human) to provide a single source of antibodies to a wide range of non-self and also self antigens without any immunization as described by Griffiths et al, in EMBO Journal 12:725-734 (1993). Furthermore, naive libraries can also be made synthetically by cloning unrearranged V-gene segments from stem cells, and using PCR primers containing random sequence to encode the highly variable CDR3 regions and to accomplish rearrangement in vitro, as described by Hoogenboom and Winter in Journal of Molecular Biology 227:381-388 (1992). Patent publications describing human antibody phage libraries include, for example: U.S. Pat. Nos. 5,750,373; 7,985,840; 7,785,903 and 8,679,490 as well as US Patent Publication Nos. 2005/0079574, 2007/0117126, 2007/0237764 and 2007/0292936.

Further examples of methods known in the art for screening combinatorial libraries for antibodies with a desired activity or activities include ribosome and mRNA display, as well as methods for antibody display and selection on bacteria, mammalian cells, insect cells of yeast cells. Methods for yeast surface display are reviewed, e.g., in Scholler et al, in Methods in Molecular Biology 503:135-56 (2012) and in Cherf et al, in Methods in Molecular biology 1319:155-175 (2015) as well as in Zhao et al, in Methods in Molecular Biology 889:73-84 (2012). Methods for ribosome display are described, e.g., in He et al, in Nucleic Acids Research 25:5132-5134 (1997) and in Hanes et al, in PNAS 94:4937-4942 (1997).

Antibodies or antibody fragments isolated from human antibody libraries are considered human antibodies or human antibody fragments herein.

### Antibody Variants

In certain aspects, amino acid sequence variants of the antibodies provided herein are contemplated. For example, in some aspects, it is desirable to alter the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding. a) Substitution. Insertion, and

### Deletion Variants

In certain aspects, antibody variants having one or more amino acid substitutions are provided. Sites of interest for substitutional mutagenesis include the CDRs and FRs. Conservative substitutions are shown in Table 7 under the heading of “preferred substitutions”. More substantial changes are provided in Table 7 under the heading of “exemplary substitutions”, and as further described below in reference to amino acid side chain classes. Amino acid substitutions may be introduced into an antibody of interest and the products screened for a desired activity, e.g., retained/improved antigen binding, decreased immunogenicity.

TABLE 7

| Original | | Preferred |
|---|---|---|
| Residues | Substitutions | Substitutions |
| Ala | Val, Leu, Ile | Val |
| Arg | Lys, Gln, Asn | Lys |
| Asn | Gln | |
| Asp | Glu | |
| Cys | Ser, Ala | Ser |
| Gln | Asn | Asn |
| Glu | Asp | Asp |
| Gly | Pro, Ala | Ala |
| His | Asn, Gln, Lys, Arg | Arg |
| Ile | Leu, Val, Met, Ala, Phe, Norleucine | Leu |
| Leu | Norleucine, Ile, Val, Met, Ala, Phe | Ile |
| Lys | Arg, Gln, Asn, 1,4 Diamino-butyric Acid | Arg |
| Met | Leu, Phe, Ile | Leu |
| Phe | Leu, Val, Ile, Ala, Tyr | Leu |
| Pro | Ala | Gly |
| Ser | Thr, Ala, Cys | Thr |
| Thr | Ser | Ser |
| Trp | Tyr, Phe | Tyr |
| Tyr | Trp, Phe, Thr, Ser | Phe |
| Val | Ile, Met, Leu, Phe, Ala, Norleucine | Leu |

Amino acids, in various aspects, are grouped according to common side-chain properties:
(1) hydrophobic: Norleucine, Met, Ala, Val, Leu, lie;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro; and
(6) aromatic: Trp, Tyr, Phe.

Non-conservative substitutions entail exchanging a member of one of these classes for a member of another class.

One type of substitutional variant involves substituting one or more hypervariable region residues of a parent antibody (e.g., a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more. CDR residues are mutated and the variant antibodies displayed on phage and screened for a particular biological activity (e.g., binding affinity). Alterations (e.g., substitutions) may be made in CDRs, e.g., to improve antibody affinity. Such alterations may be made in CDR “hotspots”. i.e., residues encoded by codons that undergo mutation at high frequency during the somatic maturation process (see, e.g., Chowdhury, Methods Mol. Biol. 207:179-196 (2008), and/or residues that contact antigen, with the resulting variant VH or VL being tested for binding affinity. Affinity maturation by constructing and reselecting from secondary libraries has been described, e.g., in Hoogenboom et al, in Methods in Molecular Biology 178:1-37 (O'Brien et al., ed., Human Press, Totowa, NJ, (2001).) In some aspects of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves CDR-directed approaches, in which several CDR residues (e.g., 4-6 residues at a time) are randomized. CDR residues involved in antigen binding may be specifically identified, e.g., using alanine scanning mutagenesis or modeling. CDR-H3 and CDR-L3 in particular are often targeted.

In certain aspects, substitutions, insertions, or deletions occur within one or more CDRs so long as such alterations do not substantially reduce the ability of the antibody to bind antigen. For example, conservative alterations (e.g., conservative substitutions as provided herein) that do not substantially reduce binding affinity may be made in the CDRs. Such alterations may, for example, be outside of antigen contacting residues in the CDRs. In certain variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as arg, asp, his, lys, and glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex may be used to identify contact points between the antibody and antigen. Such contact residues and neighboring residues may be targeted or eliminated as candidates for substitution. Variants may be screened to determine whether they contain the desired properties. Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g., for ADEPT (antibody directed enzyme prodrug therapy)) or a polypeptide which increases the serum half-life of the antibody.

In certain aspects, an antibody provided herein is altered to increase or decrease the extent to which the antibody is glycosylated. Addition or deletion of glycosylation sites to an antibody may be conveniently accomplished by altering the amino acid sequence such that one or more glycosylation sites is created or removed.

Where the antibody comprises an Fc region, the oligosaccharide attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some aspects, modifications of the oligosaccharide in an antibody of the disclosure are made in order to create antibody variants with certain improved properties.

In one aspect, antibody variants are provided having a non-fucosylated oligosaccharide, i.e. an oligosaccharide structure that lacks fucose attached (directly or indirectly) to an Fc region. Such non-fucosylated oligosaccharide (also referred to as “afucosylated” oligosaccharide) particularly is an N-linked oligosaccharide which lacks a fucose residue attached to the first GlcNAc in the stem of the biantennary oligosaccharide structure. In one aspect, antibody variants are provided having an increased proportion of non-fucosylated oligosaccharides in the Fc region as compared to a native or parent antibody. For example, the proportion of non-fucosylated oligosaccharides may be at least about 20%, at least about 40%, at least about 60%, at least about 80%, or even about 100% (i.e. no fucosylated oligosaccharides are present). The percentage of non-fucosylated oligosaccharides is the (average) amount of oligosaccharides lacking fucose residues, relative to the sum of all oligosaccharides attached to Asn 297 (e. g. complex, hybrid and high mannose structures) as measured by MALDI-TOF mass spectrometry, as described in WO 2006/082515, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such antibodies having an increased proportion of non-fucosylated oligosaccharides in the Fc region may have improved FcyRIIIa receptor binding and/or improved effector function. See, e.g., US 2003/0157108; US 2004/0093621.

Examples of cell lines capable of producing antibodies with reduced fucosylation include Lecl3 CHO cells deficient in protein fucosylation (Ripka et al. Arch. Biochem. Biophys. 249:533-545 (1986); US 2003/0157108; and WO 2004/056312, especially at Example 11), and knockout cell lines, such as alpha-1,6-fucosyltransferase gene, FUT8, knockout CHO cells (see, e.g., Yamane-Ohnuki et al. Biotech. Bioeng. 87:614-622 (2004); Kanda, Y. et al., Biotechnol. Bioeng., 94 (4): 680-688 (2006); and WO 2003/085107), or cells with reduced or abolished activity of a GDP-fucose synthesis or transporter protein (see. e.g., US2004259150, US2005031613, US2004132140, US2004110282).

In a further aspect, antibody variants are provided with bisected oligosaccharides, e.g., in which a biantennary oligosaccharide attached to the Fc region of the antibody is bisected by GlcNAc. Such antibody variants may have reduced fucosylation and/or improved ADCC function as described above. Examples of such antibody variants are described, e.g., in Umana et al., Nat Biotechnol 17, 176-180 (1999); Ferrara et al., Biotechn Bioeng 93, 851-861 (2006); WO 99/54342; WO 2004/065540, WO 2003/011878.

Antibody variants with at least one galactose residue in the oligosaccharide attached to the Fc region are also provided. Such antibody variants may have improved CDC function. Such antibody variants are described, e.g., in WO 1997/30087; WO 1998/58964; and WO 1999/22764.

In certain aspects, one or more amino acid modifications are introduced into the Fc region of an antibody provided herein, thereby generating an Fc region variant. The Fc region variant may comprise a human Fc region sequence (e.g., a human IgGi, IgG2, IgG3 or IgG4 Fc region) comprising an amino acid modification (e.g., a substitution) at one or more amino acid positions.

In certain aspects, the disclosure contemplates an antibody variant that possesses some but not all effector functions, which make it a desirable candidate for applications in which the half-life of the antibody in vivo is important yet certain effector functions (such as complement-dependent cytotoxicity (CDC) and antibody-dependent cell-mediated cytotoxicity (ADCC)) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcyR binding (hence likely lacking ADCC activity), but retains FcRn binding ability. The primary cells for mediating ADCC, NK cells, express FcyRIII only, whereas monocytes express FcyRI, FcyRII and FcyRIIF FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g., Hellstrom, I. et al. Proc. Nat'l. Acad. Sci. USA 83:7059-7063 (1986) and Hellstrom, I et al., Proc. Nat'l Acad. Sci. USA 82:1499-1502 (1985); 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (Cell Technology, Inc. Mountain View, CA; and CytoTox 960 non-radioactive cytotoxicity assay (Promega, Madison, WI). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al. Proc. Nat'l Acad. Sci. USA 95:652-656 (1998). C1q binding assays may also be carried out to confirm that the antibody is unable to bind C1q and hence lacks CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)). FcRn binding and in vivo clearance/half-life determinations, in various aspects, are performed using methods known in the art (see, e.g., Petkova, S. B. et al., Int'l. Immunol. 18 (12): 1759-1769 (2006): WO 2013/120929 AI).

Antibodies with reduced effector function include those with substitution of one or more of Fc region residues 238, 265, 269, 270, 297, 327 and 329 (U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. No. 7,332,581).

Certain antibody variants with improved or diminished binding to FcRs are described. (See, e.g., U.S. Pat. No. 6,737,056; WO 2004/056312, and Shields et al., J. Biol. Chem. 9 (2): 6591-6604 (2001).) In certain aspects, an antibody variant comprises an Fc region with one or more amino acid substitutions which improve ADCC, e.g., substitutions at positions 298, 333, and/or 334 of the Fc region (EU numbering of residues).

In certain aspects, an antibody variant comprises an Fc region with one or more amino acid substitutions which diminish FcyR binding, e.g., substitutions at positions 234 and 235 of the Fc region (EU numbering of residues). In one aspect, the substitutions are L234A and L235A (LALA). In certain aspects, the antibody variant further comprises D265A and/or P329G in an Fc region derived from a human IgGi Fc region. In one aspect, the substitutions are L234A, L235A and P329G (LALA-PG) in an Fc region derived from a human IgGi Fc region. (See, e.g., WO 2012/130831). In another aspect, the substitutions are L234A, L235A and D265A (LALA-DA) in an Fc region derived from a human IgGi Fc region.

In some aspects, alterations are made in the Fc region that result in altered (i.e., either improved or diminished) C1q binding and/or Complement Dependent Cytotoxicity (CDC), e.g., as described in U.S. Pat. No. 6,194,551. WO 99/51642, and Idusogie et al. J. Immunol. 164:4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934 (Hinton et al.). Those antibodies comprise an Fc region with one or more substitutions therein which improve binding of the Fc region to FcRn. Such Fc variants include those with substitutions at one or more of Fc region residues: 238, 252, 254, 256, 265, 272, 286, 303, 305, 307, 311, 312, 317, 340, 356, 360, 362, 376, 378, 380, 382, 413, 424, 428 or 434, e.g., substitution of Fc region residue 434 (See, e.g., U.S. Pat. No. 7,371,826; DalfAcqua, W. F., et al. J. Biol. Chem. 281 (2006) 23514-23524). In some embodiments, an antibody provided herein comprises substitutions M428L and/or N434S, such as M428L and N434S (“LS”).

Fc region residues critical to the mouse Fc-mouse FcRn interaction have been identified by site-directed mutagenesis (see e.g. DalFAcqua, W. F., et al. J. Immunol 169 (2002) 5171-5180). Residues 1253, H310, H433, N434, and H435 (EU index numbering) are involved in the interaction (Medesan, C., et al., Eur. J. Immunol. 26 (1996) 2533; Firan, M., et al., Int. Immunol. 13 (2001) 993; Kim, J. K., et al., Eur. J. Immunol. 24 (1994) 542). Residues 1253, H310, and H435 were found to be critical for the interaction of human Fc with murine FcRn (Kim, J. K., et al., Eur. J. Immunol. 29 (1999) 2819). Studies of the human Fc-human FcRn complex have shown that residues 1253, S254, H435, and Y436 are crucial for the interaction (Firan, M., et al., Int. Immunol. 13 (2001) 993; Shields, R. L., et al., J. Biol. Chem. 276 (2001) 6591-6604). In Yeung, Y. A., et al. (J. Immunol. 182 (2009) 7667-7671) various mutants of residues 248 to 259 and 301 to 317 and 376 to 382 and 424 to 437 have been reported and examined.

In certain aspects, an antibody variant comprises an Fc region with one or more amino acid substitutions, which reduce FcRn binding, e.g., substitutions at positions 253, and/or 310, and/or 435 of the Fc-region (EU numbering of residues). In certain aspects, the antibody variant comprises an Fc region with the amino acid substitutions at positions 253, 310 and 435. In one aspect, the substitutions are 1253 A, H310A and H435A in an Fc region derived from a human IgGI Fo-region. See, e.g., Grevys, A., et al., J. Immunol. 194 (2015) 5497-5508.

In certain aspects, an antibody variant comprises an Fc region with one or more amino acid substitutions, which reduce FcRn binding, e.g., substitutions at positions 310, and/or 433, and/or 436 of the Fc region (EU numbering of residues). In certain aspects, the antibody variant comprises an Fc region with the amino acid substitutions at positions 310, 433 and 436. In one aspect, the substitutions are H310A, H433 A and Y436A in an Fc region derived from a human IgGI Fc-region. (See, e.g., WO 2014/177460 AI).

In certain aspects, an antibody variant comprises an Fc region with one or more amino acid substitutions which increase FcRn binding, e.g., substitutions at positions 252, and/or 254, and/or 256 of the Fc region (EU numbering of residues). In certain aspects, the antibody variant comprises an Fc region with amino acid substitutions at positions 252, 254, and 256. In one aspect, the substitutions are M252Y, S254T and T256E in an Fc region derived from a human IgGi Fc-region. See also Duncan & Winter, Nature 322:738-40 (1988); U.S. Pat. Nos. 5,648,260; 5,624,821; and WO 94/29351 concerning other examples of Fc region variants.

The C-terminus of the heavy chain of the antibody as reported herein can be a complete C-terminus ending with the amino acid residues PGK. The C-terminus of the heavy chain can be a shortened C-terminus in which one or two of the C terminal amino acid residues have been removed. In one preferred aspect, the C-terminus of the heavy chain is a shortened C-terminus ending PG. In one aspect of all aspects as reported herein, an antibody comprising a heavy chain including a C-terminal CH3 domain as specified herein, comprises the C-terminal glycine-lysine dipeptide (G446 and K447, EU index numbering of amino acid positions). In one aspect of all aspects as reported herein, an antibody comprising a heavy chain including a C-terminal CH3 domain, as specified herein, comprises a C-terminal glycine residue (G446, EU index numbering of amino acid positions).

### d) Cysteine Engineered Antibody Variants

In certain aspects, it is desirable to create cysteine engineered antibodies, e.g., THIOMAB™ antibodies, in which one or more residues of an antibody are substituted with cysteine residues. In particular aspects, the substituted residues occur at accessible sites of the antibody. By substituting those residues with cysteine, reactive thiol groups are thereby positioned at accessible sites of the antibody and may be used to conjugate the antibody to other moieties, such as drug moieties or linker-drug moieties, to create an immunoconjugate, as described further herein. Cysteine engineered antibodies, in various aspects, are generated as described, e.g., in U.S. Pat. Nos. 7,521,541; 8,309,300; 7,855,275; or 9,000, 130: or WO 2016040856.

### Antibody Derivatives

In certain aspects, an antibody provided herein is further modified to contain additional nonproteinaceous moieties that are known in the art and readily available.

The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1, 3, 6-trioxane, ethylene/maleic anhydride copolymer, polyaminoacids (either homopolymers or random copolymers), and dextran or poly(n-vinyl pyrrolidone) polyethylene glycol, propropylene glycol homopolymers, proly propylene oxide/ethylene oxide copolymers, polyoxyethylated polyols (e.g., glycerol), polyvinyl alcohol, and mixtures thereof. Polyethylene glycol propionaldehyde may have advantages in manufacturing due to its stability in water. The polymer, in various aspects, is of any molecular weight, and, in various aspects, is branched or unbranched. The number of polymers attached to the antibody may vary, and if more than one polymer is attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, and the like.

### Recombinant Methods and Compositions

Antibodies may be produced using recombinant methods and compositions, e.g., as described in U.S. Pat. No. 4,816,567. For these methods one or more isolated nucleic acid(s) encoding an antibody are provided.

In case of a native antibody or native antibody fragment two nucleic acids are required, one for the light chain or a fragment thereof and one for the heavy chain or a fragment thereof. Such nucleic acid(s) encode an amino acid sequence comprising the VL and/or an amino acid sequence comprising the VH of the antibody (e.g., the light and/or heavy chain(s) of the antibody). These nucleic acids can be on the same expression vector or on different expression vectors.

Such nucleic acid(s) encode an amino acid sequence comprising the first VL and/or an amino acid sequence comprising the first VH including the first heteromonomeric Fc-region and/or an amino acid sequence comprising the second VL and/or an amino acid sequence comprising the second VH including the second heteromonomeric Fc-region of the antibody (e.g., the first and/or second light and/or the first and/or second heavy chains of the antibody). These nucleic acids can be on the same expression vector or on different expression vectors, normally these nucleic acids are located on two or three expression vectors, i.e. one vector can comprise more than one of these nucleic acids. For example, one of the heteromonomeric heavy chain comprises the so-called “knob mutations” (T366W and optionally one of S354C or Y349C) and the other comprises the so-called “hole mutations” (T366S, L368A and Y407V and optionally Y349C or $354C) (see, e.g., Carter, P. et al., Immunotechnol. 2 (1996) 73) according to EU index numbering.

In one aspect, isolated nucleic acids encoding an antibody as used in the methods as reported herein are provided.

In one aspect, a method of making an antibody is provided, wherein the method comprises culturing a host cell comprising nucleic acid(s) encoding the antibody, as provided above, under conditions suitable for expression of the antibody, and optionally recovering the antibody from the host cell (or host cell culture medium).

For recombinant production of an antibody, nucleic acids encoding the antibody, e.g., as described above, are isolated and inserted into one or more vectors for further cloning and/or expression in a host cell. Such nucleic acids, in various aspects, are readily isolated and sequenced using conventional procedures (e.g., by using oligonucleotide probes that are capable of binding specifically to genes encoding the heavy and light chains of the antibody) or produced by recombinant methods or obtained by chemical synthesis.

Suitable host cells for cloning or expression of antibody-encoding vectors include prokaryotic or eukaryotic cells described herein. For example, antibodies, in various aspects, are produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, K. A., In: Methods in Molecular Biology, Vol. 248, Lo. B. K. C. (ed.). Humana Press, Totowa, NJ (2003), pp. 245-254, describing expression of antibody fragments in E. coli.) After expression, the antibody, in various aspects, is isolated from the bacterial cell paste in a soluble fraction and can be further purified.

In addition to prokaryotes, eukaryotic microbes such as filamentous fungi or yeast are suitable cloning or expression hosts for antibody-encoding vectors, including fungi and yeast strains whose glycosylation pathways have been “humanized”, resulting in the production of an antibody with a partially or fully human glycosylation pattern. See Gerngross, T. U., Nat. Biotech. 22 (2004) 1409-1414; and Li, H. et al., Nat. Biotech. 24 (2006) 210-215.

Suitable host cells for the expression of (glycosylated) antibody are also derived from multicellular organisms (invertebrates and vertebrates). Examples of invertebrate cells include plant and insect cells. Numerous baculoviral strains have been identified which, in various aspects, are used in conjunction with insect cells, particularly for transfection of Spodoptera frugiperda cells.

Plant cell cultures can also be utilized as hosts. See, e.g., U.S. Pat. Nos. 5,959,177, 6,040,498, 6,420,548, 7,125,978, and 6,417,429 (describing PLANTIBODIES™ technology for producing antibodies in transgenic plants).

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293T cells as described, e.g., in Graham, F. L. et al., J. Gen Virol. 36 (1977) 59-74); baby hamster kidney cells (BHK); mouse sertoli cells (TM4 cells as described, e.g., in Mather. J. P., Biol. Reprod. 23 (1980) 243-252): monkey kidney cells (CV1): African green monkey kidney cells (VERO-76); human cervical carcinoma cells (HELA): canine kidney cells (MDCK; buffalo rat liver cells (BRL 3 A); human lung cells (W138); human liver cells (Hep G2); mouse mammary tumor (MMT 060562): TRI cells (as described, e.g., in Mather, J. P. et al., Annals N.Y. Acad. Sci. 383 (1982) 44-68): MRC 5 cells; and FS4 cells. Other useful mammalian host cell lines include Chinese hamster ovary (CHO) cells, including DHFR-CHO cells (Urlaub, G. et al., Proc. Natl. Acad. Sci. USA 77 (1980) 4216-4220); and myeloma cell lines such as Y0, NS0 and Sp2/0. For a review of certain mammalian host cell lines suitable for antibody production, see, e.g., Yazaki, P. and Wu, A. M., Methods in Molecular Biology, Vol. 248, Lo. B. K. C. (ed.). Humana Press, Totowa, NJ (2004), pp. 255-268.

In one aspect, the host cell is eukaryotic, e.g., a Chinese Hamster Ovary (CHO) cell or lymphoid cell (e.g., Y0, NS0, Sp20 cell).

### Assays

Anti-TTR antibodies provided herein, in various aspects, are identified, screened for, or characterized for their physical/chemical properties and/or biological activities by various assays known in the art.

### 1. Binding Assays and Other Assays

In one aspect, an antibody of the disclosure is tested for its antigen binding activity. e.g., by known methods such as ELISA, Western blot, etc.

In another aspect, competition assays are used to identify an antibody that competes with an antibody provided herein, such as Ab1, for binding to TTR. In certain aspects, such a competing antibody binds to the same epitope (e.g., a linear or a conformational epitope) that is bound by Ab1. In certain aspects, such a competing antibody binds to the same epitope (e.g., a linear or a conformational epitope) that is bound by Ab1. Detailed exemplary methods for mapping an epitope to which an antibody binds are provided in Morris (1996) “Epitope Mapping Protocols”, in Methods in Molecular Biology vol. 66 (Humana Press, Totowa, NJ).

In an exemplary competition assay, immobilized antigen (such as TTR monomer) is incubated in a solution comprising a first labeled antibody that binds to the antigen (e.g., Ab1. Ab2, Ab3. Ab4, Ab5, Ab6 or Ab7) and a second unlabeled antibody that is being tested for its ability to compete with the first antibody for binding to the antigen. The second antibody may be present in a hybridoma supernatant. As a control, immobilized antigen is incubated in a solution comprising the first labeled antibody but not the second unlabeled antibody. After incubation under conditions permissive for binding of the first antibody to antigen, excess unbound antibody is removed, and the amount of label associated with immobilized antigen is measured. If the amount of label associated with immobilized antigen is substantially reduced in the test sample relative to the control sample, then that indicates that the second antibody is competing with the first antibody for binding to antigen. See Harlow and Lane (1988) Antibodies: A Laboratory Manual ch. 14 (Cold Spring Harbor Laboratory, Cold Spring Harbor, NY).

### 2. Activity Assays

TTR forms aggregates, as well as monomer, tetramers and intermediate folding states. Biological activity of TTR may include, e.g., vitamin A (retinol) and thyroxine transportation throughout the body. A deleterious activity of TTR is its aggregation and formation of deposits within tissues in the body, therefore an activity of a binding antibody is TTR aggregation inhibition. Antibodies having such biological activity in vivo and/or in vitro are also provided.

In certain aspects, an antibody of the disclosure is tested for such biological activity. Assays for determining TTR aggregation activity are known in the art, and typically include incubating TTR in the presence of a fluorescent indicator and an anti-TTR antibody. A nonlimiting assay for testing whether an anti-TTR antibody inhibits TTR aggregation activity follows: A TTR aggregation assay may be performed at room temperature in a fluorimeter monitoring a shift in fluorescence of the dye thioflavin ThT. A number of ways to initiate aggregation of TTR are known, including lowering pH of the solution below 4, or using trypsin-treatment. In Example 5, mTTR at a concentration of 10 UM was treated with trypsin in a 96-well fluorescent plate reader and its aggregation was monitored with ThT over time.

### Methods and Compositions for Diagnostics and Detection

In certain aspects, one or more of any of the antibodies provided herein is useful for detecting the presence of antigen in a biological sample. The term “detecting” as used herein encompasses quantitative or qualitative detection. In certain aspects, a biological sample comprises a cell or tissue, such as a blood sample.

In one aspect, an antibody for use in a method of diagnosis or detection is provided. In a further aspect, a method of detecting the presence of TTR monomer in a biological sample is provided. In certain aspects, the method comprises contacting the biological sample with an anti-TTR antibody as described herein under conditions permissive for binding of the antibody to its antigen, and detecting whether a complex is formed between the antibody and the antigen. Such method may be an in vitro or using in vivo blood or tissues samples. In some embodiments, methods of selecting patients for treatment with an antibody provided herein comprise determining levels of TTR species (such as monomer, tetramer, or aggregates) in a sample from the patient.

In certain aspects, labeled anti-TTR antibodies are provided in any form of an immunoassay. Labels include, but are not limited to, labels or moieties that are detected directly (such as fluorescent, chromophoric, electron-dense, chemiluminescent, and radioactive labels), as well as moieties, such as enzymes or ligands, that are detected indirectly, e.g., through an enzymatic reaction or molecular interaction. Exemplary labels include, but are not limited to, the radioisotopes 32P, 14C, 1251, 3H, and 1311, fluorophores such as rare earth chelates or fluorescein and its derivatives, rhodamine and its derivatives, dansyl, umbelliferone, luceriferases, e.g., firefly luciferase and bacterial luciferase (U.S. Pat. No. 4,737,456), luciferin, 2,3-dihydrophthalazinediones, horseradish peroxidase (HRP), alkaline phosphatase, b-galactosidase, glucoamylase, lysozyme, saccharide oxidases, e.g., glucose oxidase, galactose oxidase, and glucose-6-phosphate dehydrogenase, heterocyclic oxidases such as uricase and xanthine oxidase, coupled with an enzyme that employs hydrogen peroxide to oxidize a dye precursor such as HRP, lactoperoxidase, or microperoxidase, biotin/avidin, spin labels, bacteriophage labels, stable free radicals, and the like.

Use of the anti-TTR monomer antibodies in combination, such as one for capture and another for detection, can be an effective diagnostic. Quanterix has developed an approach for detecting thousands of single protein molecules simultaneously for ultra-sensitive detection of binding. Utilizing the same reagents as a conventional ELISA, this method has been used to measure proteins in a variety of different matrices (serum, serum/plasma, cerebral spinal fluid, urine, cell extracts etc.) at femtomolar (fg/ml) concentrations, offering a roughly 1000-fold improvement in sensitivity. This approach makes use of arrays of femtoliter-sized reaction chambers, termed single-molecule arrays (Simoa™) that can isolate and detect single enzyme molecules. Because the array volumes are approximately 2 billion times smaller than a conventional ELISA, a rapid buildup of fluorescent product is generated if a labeled protein is present. With diffusion defeated, this high local concentration of product can be readily observed. Only one single molecule is needed to reach the detection limit.

In the first step of this single-molecule immunoassay, antibody capture agents are attached to the surface of paramagnetic beads (2.7 □m diameter) that will be used to concentrate a dilute solution of molecules. The beads typically contain approximately 250,000 attachment sites so one can think of each bead as having a “lawn” of capture molecules. The beads are added to the sample solution such that there are many more beads than target molecules. Typically 500,000 beads will be added to a 100 μL sample. There are two advantages for adding so many beads. First, at a roughly 10:1 bead to molecule ratio, the percentage of beads that contain a labeled immunocomplex follows a Poisson distribution. At low concentrations of protein, the Poisson distribution indicates that each bead will capture either a single immunocomplex or none. For example, if 1 fM of a protein in 0.1 ml (60,000 molecules) is captured and labeled on 500.000 beads, then 12% of the beads will carry one protein molecule and 88% will not carry any protein molecules. Second, with so many beads in solution, the bead-to-bead distance is small, such that every molecule encounters a bead in less than a minute. Diffusion of the target analyte molecules, even large proteins, occurs on a time scale such that all the molecules should in theory quickly have multiple collisions with multiple beads. In this manner, the slow binding to a fixed capture surface is avoided and the efficiency of binding increases dramatically. Beads are then washed to remove nonspecifically bound proteins, incubated with biotinylated detection antibody and then with β-galactosidase labeled streptavidin. In this manner, each bead that has captured a single protein molecule is labeled with an enzyme. Beads that do not capture a molecule remain label free.

Rather than ensemble readout, beads are loaded into arrays of 216,000 femtoliter-sized wells that have been sized to hold no more than one bead per well (4.25 μm width, 3.25 Om depth). Beads are added in the presence of substrate, and wells are subsequently sealed with oil and imaged. Simoa permits the detection of very low concentrations of enzyme labels by confining the fluorophores generated by individual enzymes to extremely small volumes (˜40 fL), ensuring a high local concentration of fluorescent product molecules. If a target analyte has been captured (immunocomplex formed), then the substrate will be converted to a fluorescent product by captured enzyme label. The ratio of the number of wells containing a bead with an enzyme label to the total number of wells containing a bead corresponds to the analyte concentration in the sample. By acquiring two fluorescence images of the array, it is possible to demonstrate an increase in signal thereby confirming the presence of a true immunocomplex, and those beads associated with a single enzyme molecule (“on” well) can be distinguished from those not associated with an enzyme (“off” well). The protein concentration in the test sample is determined by counting the number of wells containing both a bead and fluorescent product relative to the total number of wells containing beads. As Simoa enables concentration to be determined digitally rather than by using the total analog signal, this approach to detecting single immunocomplexes has been termed digital ELISA. The ability of digital ELISA to measure much lower concentrations of proteins than conventional ELISA derives from two effects: (i) the high sensitivity of Simoa to enzyme label and (ii) the low background signals that can be achieved by digitizing the detection of proteins. For antibodies of given affinity, the sensitivity of the immunoassay will be determined by the assay background. The high label sensitivity and decreased label concentration helps reduce nonspecific binding to the capture surface, resulting in much lower background signals.

### Pharmaceutical Compositions

In a further aspect, the disclosure provides a composition comprising any of the antibodies provided herein, e.g., for use in a therapeutic method or for use in a medicament as described herein. In one aspect, a composition comprises any of the antibodies provided herein and a pharmaceutically acceptable carrier. In some aspects, therefore, a composition of the disclosure is a pharmaceutical composition. In another aspect, a pharmaceutical composition comprises any of the antibodies provided herein and at least one additional therapeutic agent, e.g., as described herein below.

Compositions or pharmaceutical compositions comprising an anti-TTR antibody as described herein are prepared by mixing such antibody or antibodies having the desired degree of purity with one or more optional pharmaceutically acceptable carriers {Remington's Pharmaceutical Sciences 16th edition, Osol, A. Ed. (1980)), in the form of lyophilized compositions or aqueous solutions. Pharmaceutically acceptable carriers are generally nontoxic to recipients at the dosages and concentrations employed, and include, but are not limited to: buffers such as histidine, phosphate, citrate, acetate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides: proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g., Zn-protein complexes); and/or non-ionic surfactants such as polyethylene glycol (PEG). Exemplary pharmaceutically acceptable carriers herein further include interstitial drug dispersion agents such as soluble neutral-active hyaluronidase glycoproteins (sHASEGP), for example, human soluble PH-20 hyaluronidase glycoproteins, such as rHuPH20 (HYLENEX®, Halozyme, Inc.). Certain exemplary sHASEGPs and methods of use, including rHuPH20, are described in US Patent Publication Nos. 2005/0260186 and 2006/0104968. In one aspect, a sHASEGP is combined with one or more additional glycosaminoglycanases such as chondroitinases.

In some embodiments, an antibody or antibodies provided herein are formulated for subcutaneous administration. In some embodiments, an antibody or antibodies provided herein are formulated for intravenous administration. In some embodiments, an antibody or antibodies provided herein are formulated for topical administration.

Exemplary lyophilized antibody compositions are described in U.S. Pat. No. 6,267,958. Aqueous antibody compositions include those described in U.S. Pat. No. 6,171,586 and WO 2006/044908, the latter compositions including a histidine-acetate buffer.

The pharmaceutical composition herein may also contain more than one active ingredients as necessary for the particular indication being treated, preferably those with complementary activities that do not adversely affect each other. Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol. A. Ed. (1980).

Pharmaceutical compositions for sustained release may be prepared. Suitable examples of sustained-release preparations include semipermeable matrices of solid hydrophobic polymers containing the antibody, which matrices are in the form of shaped articles, e.g., films, or microcapsules.

The pharmaceutical compositions to be used for in vivo administration are generally sterile. Sterility may be readily accomplished, e.g., by filtration through sterile filtration membranes.

### Therapeutic Methods and Routes of Administration

Any of the antibodies provided herein may be used in therapeutic methods, alone or in combination.

In one aspect, an anti-TTR antibody for use as a medicament is provided. In further aspects, such antibodies for use in treating TTR amyloidosis causing neuropathy or cardiomyopathy are provided. In certain aspects, an anti-TTR antibody for use in a method of treatment is provided. In certain aspects, the disclosure provides such antibodies for use in a method of treating an individual having cardiomyopathy or neuropathy comprising administering to the individual an effective amount of the antibody or antibodies. In one such aspect, the method further comprises administering to the individual an effective amount of at least one additional therapeutic agent (e.g., one, two, three, four, five, or six additional therapeutic agents), e.g., as described below.

In further aspects, the disclosure provides an anti-TTR antibody for use in reducing levels of TTR folding intermediates within the blood or tissue. In certain aspects, the disclosure provides an anti-TTR antibody for use in a method of reducing TTR folding intermediates in an individual comprising administering to the individual an effective amount of the antibody or antibodies to reduce aggregation of TTR within tissues. An “individual” according to any of the above aspects is preferably a human.

In a further aspect, the disclosure provides for the use of an anti-TTR antibody in the manufacture or preparation of a medicament. In one aspect, the medicament is for treating comprising administering to an individual having the condition of TTR amyloidosis an effective amount of the medicament. In one such aspect, the method further comprises administering to the individual an effective amount of at least one additional therapeutic agent, e.g., as described below. In a further aspect, the medicament is for reducing aggregated TTR within tissues. In a further aspect, the medicament is for use in a method of reducing aggregated TTR within tissues in an individual comprising administering to the individual an effective amount of the medicament to reduce TTR folding intermediates. An “individual” according to any of the above aspects may be a human.

In a further aspect, the disclosure provides a method for treating TTR amyloidosis. In one aspect, the method comprises administering to an individual having aggregates of TTR in the heart or peripheral nerves an effective amount of an anti-TTR antibody. In one such aspect, the method further comprises administering to the individual an effective amount of at least one additional therapeutic agent, as described below.

In a further aspect, the disclosure provides pharmaceutical compositions comprising any of the antibodies provided herein, e.g., for use in any of the above therapeutic methods. In one aspect, a pharmaceutical composition comprises any of the antibodies provided herein and a pharmaceutically acceptable carrier. In another aspect, a pharmaceutical composition comprises any of the antibodies provided herein and at least one additional therapeutic agent, e.g., as described below.

Antibodies of the disclosure can be administered alone or used in a combination therapy. For instance, the combination therapy includes administering an antibody of the disclosure and administering at least one additional therapeutic agent (e.g. one, two, three, four, five, or six additional therapeutic agents). In certain aspects, the combination therapy comprises administering an antibody of the disclosure and administering at least one additional therapeutic agent, such as agents known in the TTR treatment space, a “silencer” or “stabilizer”. In some embodiments, the agent is administered orally. Such agents include, but are not limited to the stabilizers, tafamadis (VYNDAMAX™, Pfizer Inc.) or tafamidis meglumine (VYNDAQEL®, Pfizer Inc.), or the silencer, patisiran (Onpattro®, Alnylam).

Such combination therapies noted above encompass combined administration (where two or more therapeutic agents are included in the same or separate pharmaceutical compositions), and separate administration, in which case, administration of the antibody of the disclosure can occur prior to, simultaneously, and/or following, administration of the additional therapeutic agent or agents. In one aspect, administration of the antibody or antibodies of the disclosure and administration of an additional therapeutic agent occur within about one month, or within about one, two or three weeks, or within about one, two, three, four, five, or six days, of each other. In one aspect, the antibody and additional therapeutic agent are administered to the patient on Day 1 of the treatment.

An antibody of the disclosure (and any additional therapeutic agent) can be administered by any suitable means, including parenteral, intrapulmonary, and intranasal, and, if desired for local treatment, intralesional administration. Parenteral infusions include intramuscular, intravenous, intraarterial, intraperitoneal, or subcutaneous administration. Dosing can be by any suitable route, e.g., by injections, such as intravenous or subcutaneous injections, depending in part on whether the administration is brief or chronic. Various dosing schedules including but not limited to single or multiple administrations over various time-points, bolus administration, and pulse infusion are contemplated herein.

In some embodiments, an antibody of the disclosure is administered subcutaneously. In some embodiments, the antibody is administered every four weeks or every month. In some embodiments, an antibody of the disclosure is administered intravenously. In some embodiments, the antibody is administered every four weeks or every month. In some embodiments, for example, when the antibody comprises half-life extending substitutions such as M428L (EU numbering) and N434S (EU numbering), the antibody is administered every eight weeks.

Antibodies of the disclosure would be formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The antibody need not be, but is optionally formulated with one or more agents currently used to prevent or treat the disorder in question. The effective amount of such other agents depends on the amount of antibody present in the pharmaceutical composition, the type of disorder or treatment, and other factors discussed above. These are generally used in the same dosages and with administration routes as described herein, or about from 1 to 99% of the dosages described herein, or in any dosage and by any route that is empirically/clinically determined to be appropriate.

For the prevention of treatment of disease, the appropriate dosage of an antibody of the disclosure (when used alone or in combination with one or more other additional therapeutic agents) will depend on the type of disease to be treated, the type of antibody, the severity and course of the disease, whether the antibody is administered for preventive or therapeutic purposes, previous therapy, the patient's clinical history and response to the antibody, and the discretion of the attending physician. The antibody is suitably administered to the patient at one time or over a series of treatments. Depending on the type and severity of the disease, about 1 pg/kg to 15 mg/kg (e.g., 0.1 mg/kg-10 mg/kg) of antibody can be an initial candidate dosage for administration to the patient, whether, for example, by one or more separate administrations, or by continuous infusion. One typical daily dosage might range from about 1 pg/kg to 100 mg/kg or more, depending on the factors mentioned above. For repeated administrations over several days or longer, depending on the condition, the treatment would generally be sustained until a desired suppression of disease symptoms occurs. One exemplary dosage of the antibody would be in the range from about 0.05 mg/kg to about 10 mg/kg. Thus, one or more doses of about 0.5 mg/kg, 2.0 mg/kg, 4.0 mg/kg or 10 mg/kg (or any combination thereof) may be administered to the patient. Such doses may be administered intermittently, e.g., every week or every three weeks (e.g., such that the patient receives from about two to about twenty, or, e.g., about six doses of the antibody). An initial higher loading dose, followed by one or more lower doses may be administered. However, other dosage regimens may be useful. The progress of this therapy is easily monitored by conventional techniques and assays.

### Articles of Manufacture

In another aspect of the disclosure, an article of manufacture containing materials useful for the treatment, prevention and/or diagnosis of the disorders described above is provided. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating, preventing and/or diagnosing the condition and may have a sterile access port (for example the container may be an intravenous solution bag or a vial having a stopper pierceable by a hypodermic injection needle). At least one active agent in the composition is an antibody of the disclosure. The label or package insert indicates that the composition is used for treating the condition of choice. Moreover, the article of manufacture may comprise (a) a first container with a composition contained therein, wherein the composition comprises an antibody of the disclosure; and (b) a second container with a composition contained therein, wherein the composition comprises a further cytotoxic or otherwise therapeutic agent. The article of manufacture in this aspect of the disclosure may further comprise a package insert indicating that the compositions can be used to treat a particular condition. Alternatively, or additionally, the article of manufacture may further comprise a second (or third) container comprising a pharmaceutically-acceptable buffer, such as bacteriostatic water for injection (BWFI), phosphate-buffered saline, Ringer's solution and dextrose solution. It may further include other materials desirable from a commercial and user standpoint, including other buffers, diluents, filters, needles, and syringes.

### EXAMPLES

The disclosure is further described in the following examples, which do not limit the scope of the subject matter described in the claims.

### Example 1: Preparation of His-Tagged Human TTR Forms

Monomeric forms of human transthyretin containing two point mutations, Phe87Met and Leu110Met, shown in the sequence set out below, and as described in in Jiang et al., Biochemistry, 2001, 40, 11442-52, were used for antibody discovery and subsequent in vitro characterization. Codon optimization was performed on the native human gene for optimal expression in E. coli. Two constructs were created. The first consisted of the mutated sequence with the addition of an N-terminal protease cleavable six histidine affinity tag MGSSHHHHHHSSGLVPAGSHMGPTGTGESKCPLMVKVLDAVRGSPAINVAVHVFRKAAD DTWEPFASGKTSESGELHGLTTEEEFVEGIYKVEIDTKSYWKALGISPMHEHAEVVFTANDS GPRRYTIAAMLSPYSYSTTAVVTNPKE ((SEQ ID NO: 76); His_F87M L110M TTR). The second construct consisted of the same gene sequence with the addition of the amino acid sequence -GLNDIFEAQKIEWHE- (SEQ ID NO: 77) inserted between the histidine tag and protease cleavage site. This sequence, i.e., (MHHHHHHSGMSGLNDIFEAQKIEWHEGENLYFQGMGPTGTGESKCPLMVKVLDAVRGSP AINVAVHVFRKAADDTWEPFASGKTSESGELHGLTTEEEFVEGIYKVEIDTKSYWKALGISP MHEHAEVVFTANDSGPRRYTIAAMLSPYSYSTTAVVTNPKE (SEQ ID NO: 78); Avi_mTTR—in vivo biotinylation construct) allowed for in vivo biotinylation by overexpression of the biotin ligase BirA. These constructs were ligated into the pET-28a expression plasmid and then transformed into BL21 (DE3) expression cells. A second plasmid for BirA expression was co-transformed into the cells when appropriate for the in cell biotinylation. Transformed cells were grown in flasks at 37° C. until the density reached OD600 is 0.8-1.0. Recombinant protein expression was induced with isopropylthio-β-galactoside. The cultures were incubated for approximately 16 hours. Cells were then pelleted and stored for purification.

Biotinylation was achieved similar to the method of Fairfield and Howarth [Methods Mol Biol. 2015; 1266:171-184.]. For in vivo biotinylation, biotin was added to the cultures at the time of induction and the cultures incubated at 30° C. for approximately 16 hours. Collected cells were lysed in a buffer consisting of Tris pH 8.0, 100 mM NaCl and protease inhibitors. Cells were lysed by sonication and centrifuged to remove all insoluble material. The soluble fraction of the cell lysate was initially purified by immobilized metal affinity chromatography. Bound protein was eluted with a gradient of 30-500 mM imidazole. Elution fractions containing recombinant protein were concentrated and applied to a size exclusion chromatography column for final purification and buffer exchange into a buffer of 25 mM Tris and 100 mM NaCl. Fractions containing the recombinant protein were pooled, concentrated, frozen in liquid nitrogen and stored at −80° C. for later use.

Additionally, the following forms were expressed by this method:
TTR F87M L110M (monomeric TTR, mTTR) (SEQ ID NO: 80),
TTR S112I (dimer TTR, dTTR) (SEQ ID NO: 148),
TTR T119M (stabilized tetrameric TTR) (SEQ ID NO: 153),
wild-type TTR (wtTTR),
TTR D38A, F87M, L110M (SEQ ID NO: 150),
TTR F87M L110M V1221 (SEQ ID NO: 151),
TTR F87M L110M S112I (SEQ ID NO: 152),
TTR V30M F87M L110M (SEQ ID NO: 149),
and biotinylated forms of the above.

### Example 2: Preparation of Untagged Anti-Human TTR Monomer

TTR monomer was prepared by cleaving off the His-biotin tag from the His-biotin-TTR monomer prepared in Example 1, using His-AcTEV protease. The cleaved monomer was collected from the flow-through and tested for purity through a SDS-PAGE gel stained with Coomassie and in both NAVIndirect and Sandwich ELISAs.

To a 15-ml conical flask was add 3×625 μL aliquots of His-biotin-TTR monomer from Example 1 (stock @ 383 μM) for a total of 12.88 mg. AcTEV Protease (Invitrogen 12575-015) (100 μL, 1,000 units) was added and the flask was shaken at 300 RPM at 34° C. overnight. A HisTrap (nickel column) was used to remove the cleaved His-biotin tag, any uncleaved biotinylated monomer TTR, and His-AcTEV protease with a flow rate of about ˜1 mL/min (1 drop/2 seconds). After equilibrating the column with 5 CV (5 mL) of binding buffer (25 mM Tris pH 8.0, 50 μM NaCl), the cleaved monomer TTR (˜2 mL) was added. The column was eluted with 5 CV (5 mL) elution buffer (20 mM MES pH 6.8, 100 mM NaCl, 0.5 M Imidazole). The fractions were analyzed on SDS-PAGE gel and stained with Coomassie. The fractions of cleaved monomeric TTR were stored at 4° C. The fractions were combined, filtered (0.22 mm) and analyzed via UV [Nanodrop]. Final monomer (7.54 mg) was snap frozen in liquid nitrogen and stored at −80° C.

Additionally, the following forms of TTR were purified by this method:
TTR F87M L110M (monomeric TTR, mTTR) (SEQ ID NO: 80),
TTR S112I (dimer TTR, dTTR) (SEQ ID NO: 148),
TTR T119M (stabilized tetrameric TTR) (SEQ ID NO: 153),
wild-type TTR (wtTTR),
TTR D38A, F87M, L110M (SEQ ID NO: 150),
TTR F87M L110M V1221 (SEQ ID NO: 151),
TTR F87M L110M S112I (SEQ ID NO: 152),
TTR V30M F87M L110M (SEQ ID NO: 149),
and biotinylated forms of the above.

### Example 3: Generation of Antibodies for Binding the Monomeric Form of Human TTR

Murine monoclonal antibodies were discovered that had a specificity for the monomer form of transthyretin (TTR) from a single B cell screening process at Abveris. Antibodies against the monomeric form of human TTR were generated by selection of clones having high binding affinities, using the Berkeley Lights Beacon® platform. For the isolation of anti-TTR monomer antibodies, standard maturation panning strategies were performed using a solid phase panning approach. In particular, eight Abveris DiversimAb™ mice were immunized with the monomeric form of human TTR by injecting 10 μg of TTR monomer from Example 2, diluted in adjuvant intraperitoneally, in a final volume of 200 μL. Every 2-3 days later, a new immunization boost was performed, injecting 10 μg of antigen diluted in adjuvant, in a final volume of 200 μL. After two weeks, the levels of reactivity were detected in ELISA assays.

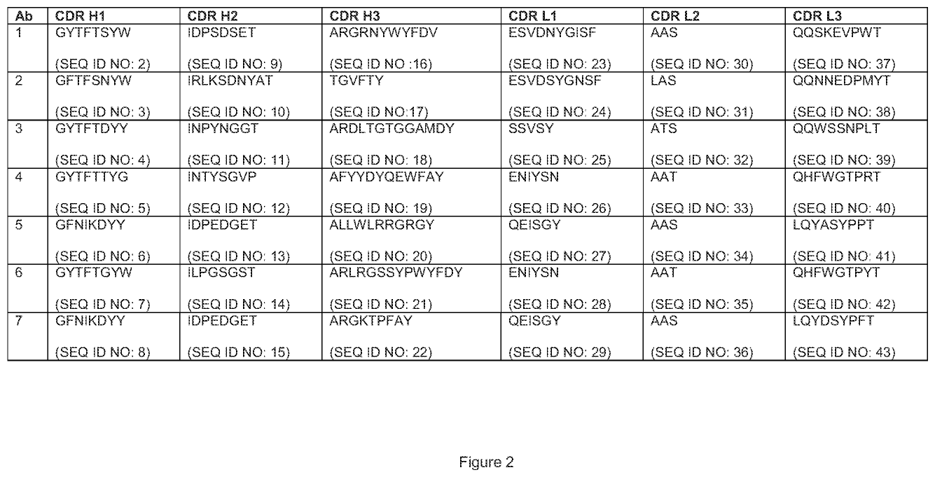

Plasma B cells were isolated from 2 mice which produced the TTR monomer binding antibodies. The animals were sacrificed, blood was collected and spleens were removed to perform fusion. The B cells were screened on the Beacon® platform. B-cells were loaded on 4 14K OPTOSelect chips via the Berkeley Lights Beacon® platform. Single cell sequencing provided paired VH/VL. Titer positive mice were selected and lymph nodes and/or spleens were harvested. In addition, a terminal bleed provided polyclonal serum. Forty-one unique antibodies were sequenced which were monomer binders and did not bind to tetrameric TTR. Small scale antibody recombinant production provided clones for confirmation of specificity and affinity. Analysis by biolayer interferometry (monomer or tetramer immobilized) and ELISA (monomer, monomer in human serum background, tetramer, tetramer in human serum background, peptides against monomer mutations) against crude antibody supernatant identified antibodies specific for monomeric TTR. Seven antibodies were purified and analyzed by biolayer interferometry and ELISAs. The CDR sequences of the monoclonal Abs antibodies are provided in FIG. 2.

### Example 4: Monomeric TTR mAb Analysis Via Indirect ELISA Screen Assay

Clear 384-well high-binding ELISA plates were coated with Neutravidin and blocked overnight. On the day of the ELISA, the plate was washed with PBS-T [PBS+0.05% Tween20] using a robotic liquid handler. Biotinylated TTR forms from Example 1 (monomer, dimer, stabilized tetramer, or wild-type TTR) were added and incubated. The plate was washed again with PBS-T. and the antibodies were added and incubated. The plate was washed again with PBS-T and the HRP-conjugated anti-species antibodies (anti-mouse-HRP) was added and incubated. The plate was washed a final time and TMB substrate (Biolegend) was added. A phosphoric acid solution (1M) was added to stop the reaction. The plate was read at two absorbance wavelengths (450 nm and 570 nm). The EC₅₀s were calculated and are provided in Table 8. All mAbs appeared monomer specific in indirect ELISA using NAV coated plate capture of antigen. See FIGS. 3A-C and 4A-C.

TABLE 8
mTTR binding EC₅₀'s

| Antibody | EC₅₀ (nM) |
|---|---|
| 1 | 0.20 |
| 2 | 1.45 |
| 3 | 14.07 |
| 4 | 0.11 |
| 5 | 0.20 |
| 6 | 0.45 |
| 7 | 4.22 |

### Example 5: Monomeric TTR Aggregation Assay with mAb Dose Response

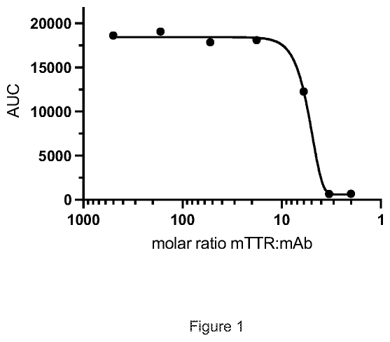

Prior to the experiment, an anti-monomeric TTR mAb (Ab1) was dialyzed into PBS. Then, the anti-mTTR mAb was serially diluted in PBS in a 96-well plate. Thioflavin T (ThT) was added for a final concentration of 50 μM, and sodium azide was added for a final concentration of 0.01%. Recombinant mTTR protein was prepared by a very brief trypsin digest in PBS. Trypsin digestion was arrested by addition of phenylmethylsulfonyl fluoride (PMSF). The trypsin-treated mTTR was added to the 96-well plate to a final concentration of 10 μM for a final volume of 200 μL. The 96-well plate was sealed and ThT fluorescence signal (442 nm ex., 485 nm em.) was monitored in a Tecan GENios fluorescence reader for 5 days at 37° C. with continuous shaking between measurements. See FIG. 1. Inhibition of mTTR aggregation was observed with concentrations above 0.19 μM.

### Example 6: Humanization of mAB TTR Antibodies

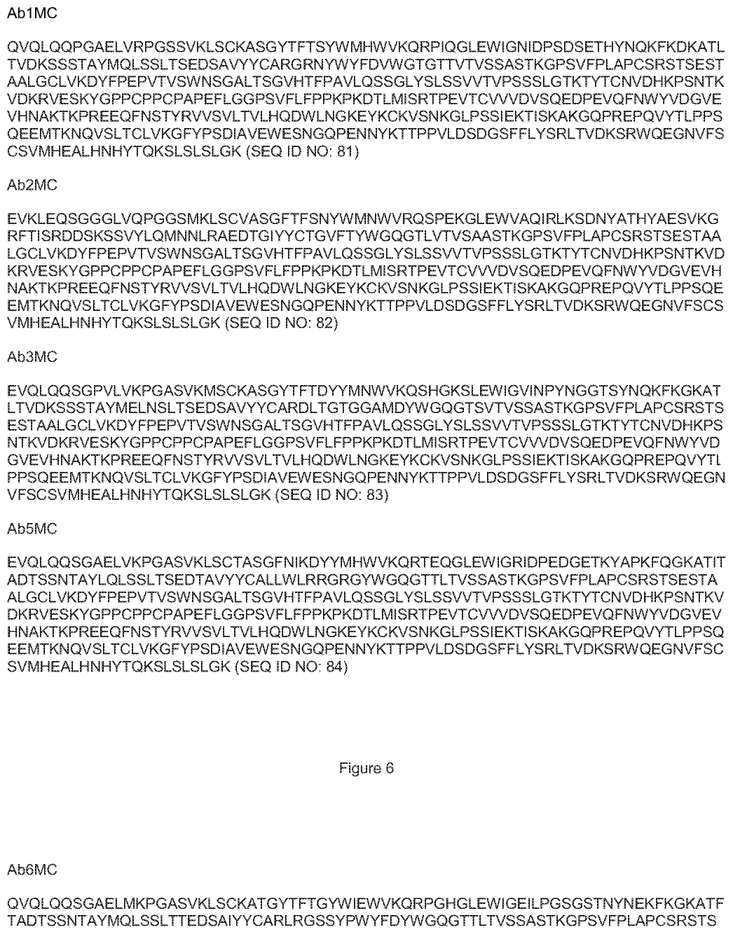

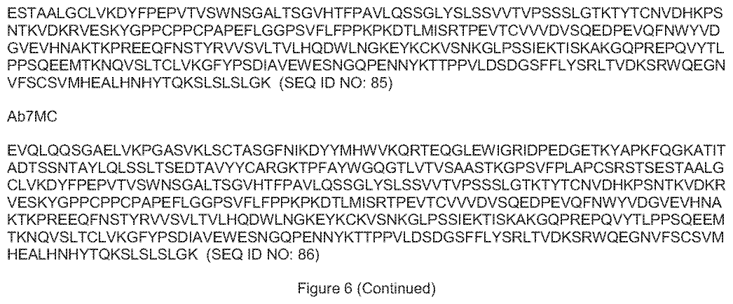

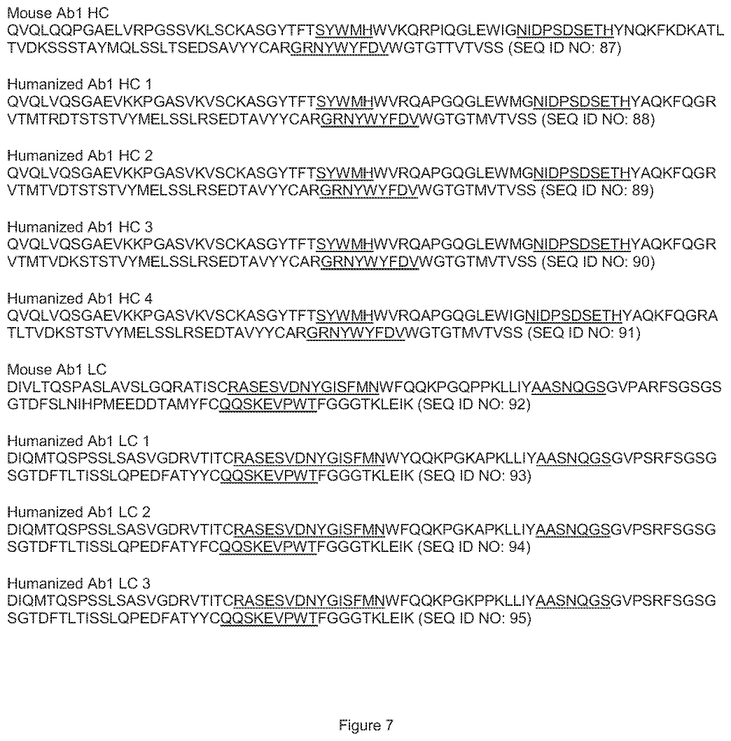

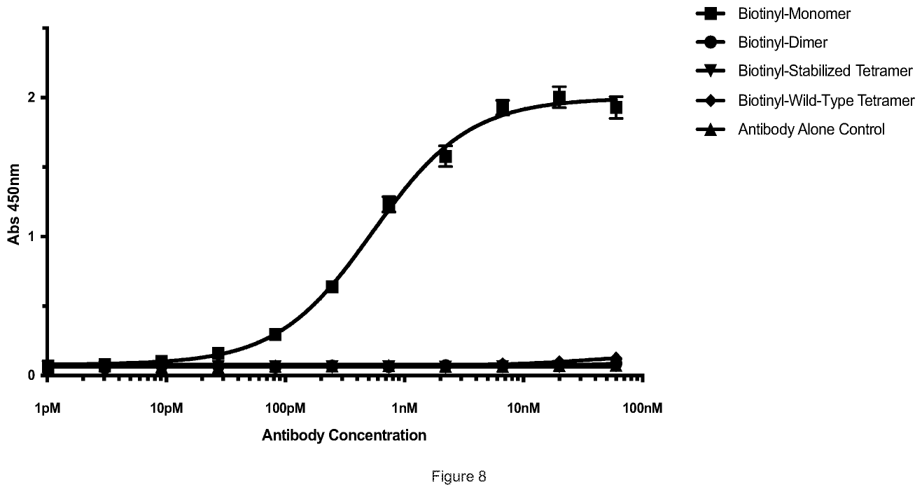

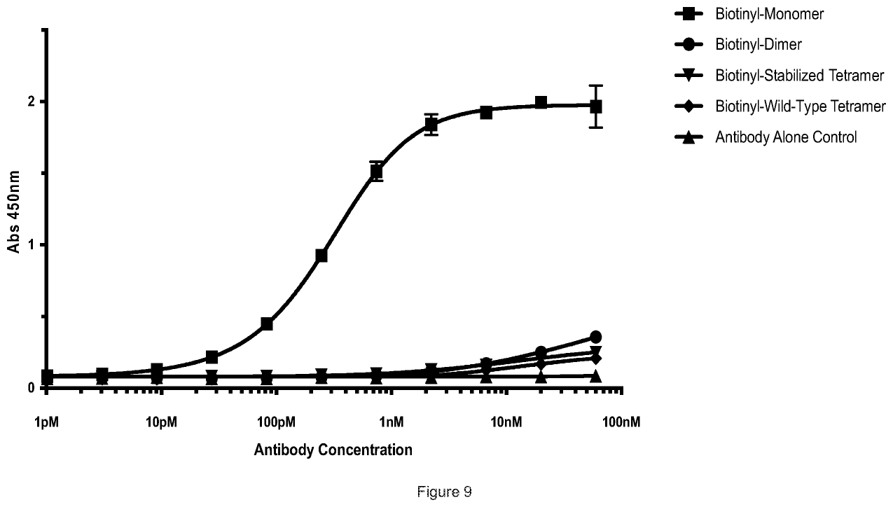

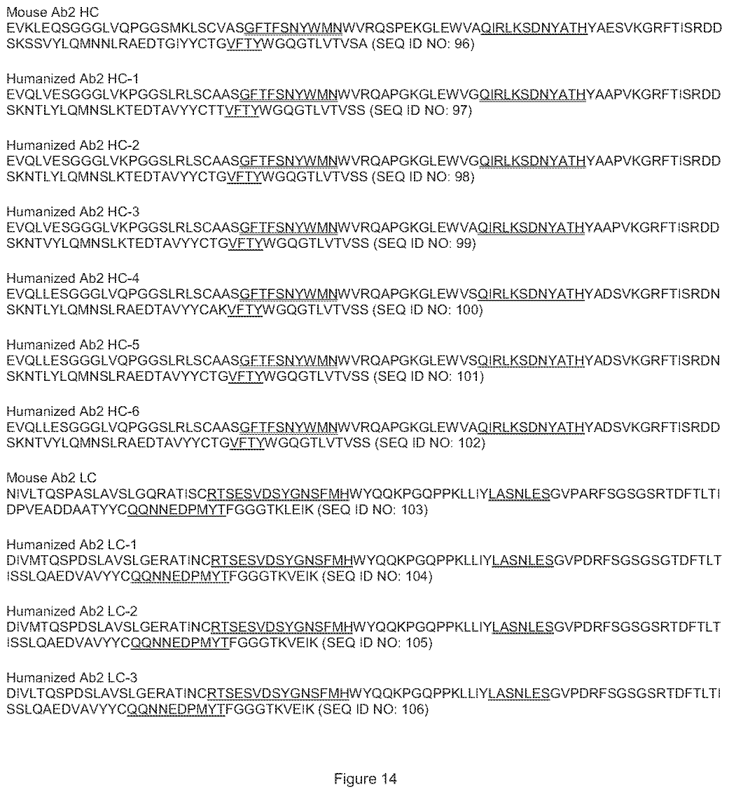

mAbs 1 and 2 were optimized via various humanization techniques. For example, mouse variable/hIgG4 chimeric sequences were first prepared. Subsequently, humanized CDRs+LV (HC), CDRX+LV, S (LC) were prepared. The sequences of the humanized mAbs antibodies are provided in FIGS. 6-9, 14, and 18-21. The humanized antibodies do not self-aggregate, and are producible in high quantities by cellular systems.

### Example 7: Humanized mAb TTR Antibodies Retain Binding Potency

The various antibody chimeras and constructs from Example 6 were analyzed by the indirect ELISA assay, as described in Example 4, where the biotinyl-mTTR were bound to avidin plates. The EC₅₀s were calculated and are provided in Table 9. The humanized construct for Ab1 showed similar affinity as the chimera form of Ab1, although it was about 10 fold weaker than the IgG2 form. The humanized version of Ab2 showed similar affinity to the IgG2 form.

TABLE 9

| Antibody | EC₅₀ (nM) |
|---|---|
| #Ab1 | 0.04 |
| mouse variable/hKappa LC/hIgG4 | 0.30 |
| HC background Chimera | |
| Humanized Ab1 construct | 0.55 |
| #Ab2 | 0.16 |
| Humanized Ab2 construct | 0.30 |

### Example 8: Humanized mAb TTR Antibodies Retain Binding Specificity

The various antibody chimeras and constructs described in Example 6 were analyzed by the indirect ELISA assay described in Example 4. The humanized antibodies retained specificity to mTTR over stabilized tetrameric TTR. The specificities of humanized Ab1 and Ab2 are provided in FIGS. 9 and 10, respectively.

### Example 9: Determination of Dissociation Constant (K_{d}) Via Biolayer Interferometry

The mAbs from Example 2 were analyzed by the proprietary biolayer interferometry assay [Octet RED, Forte Bio] at Abveris. In brief, individual anti-mouse antibody biosensors were loaded with mlgG from supernatant. A baseline was measured for each biosensor. The antibody-loaded biosensors were then allowed to bind biotinylated monomer from Example 2 to measure the association rate. Biosensors were moved to a buffer well to measure the monomer dissociation rate and together with the association rate determine the dissociation constant (Kd). Biosensors were regenerated for additional measurements, such as EC₅₀s. A baseline was measured for each biosensor. The antibody-loaded biosensors were then allowed to bind biotinylated stabilized tetramer TTR (made similar to the method described in Example 1) to measure the tetramer association rate. Biosensors were moved to a buffer well to measure the tetramer dissociation rate and together with the association rate determine the dissociation constant (Kd).

The Kd's calculated via biolayer interferometry are provided in Table 10.

TABLE 10

| | | K_{D} in nM |
|---|---|---|
| | K_{D} in nM | TTR stabilized |
| | TTR monomer | tetramer |
| Antibody | Immobilized | immobilized |
| 1 | 2.06 | not observed |
| 2 | 6.21 | not observed |
| 3 | 12.05 | not observed |
| 4 | 5.03 | 3.62 |
| 5 | 1.31 | 9.49 |
| 6 | 11.44 | not observed |
| 7 | not observed | not observed |

### Example 10: Epitope Binding with Monomeric TTR mAb Analysis Via Indirect ELISA Using Linear Peptides

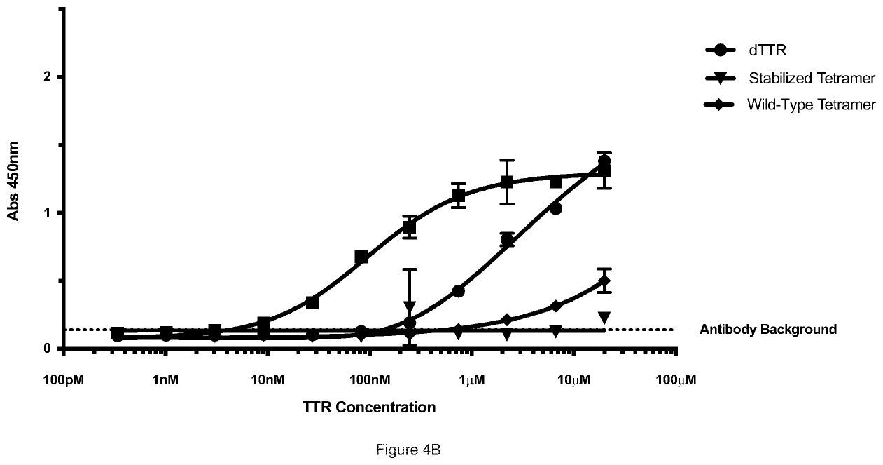

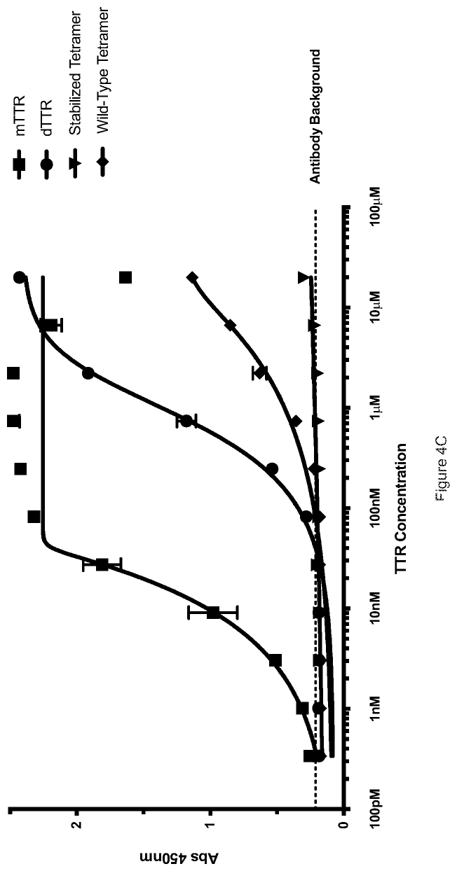

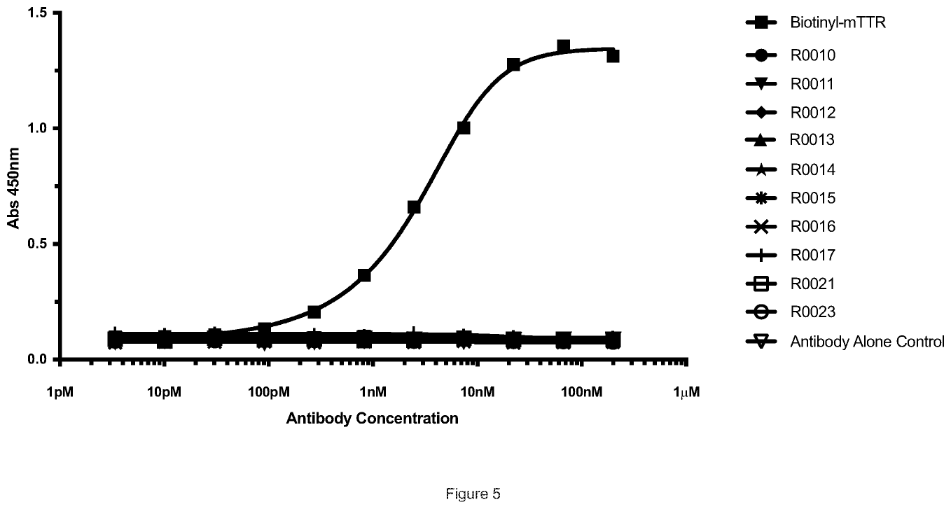

An indirect format ELISA was used for detection of biotinylated peptides by the Abs of the disclosure. Clear 384-well high-binding ELISA plates were coated with Neutravidin and blocked overnight. The plate was washed with PBS-T [PBS+0.05% Tween20] using a robotic liquid handler. The biotinylated-linear peptides [GPTGTGESKCPL (R0017) (SEQ ID NO: 65); MVKVLDAVRGSPA (R0010, SEQ ID NO: 66); PFHEHAEVVFTA (R0011, SEQ ID NO: 67); EHAEVVFTA (R0016, SEQ ID NO: 68); YTIAALLS (R0012, SEQ ID NO: 69); LSPYSY (R0014, SEQ ID NO: 70); SYSTTAVV (R0013, SEQ ID NO: 71); YTIAALLSPYSYSTTAVV (R0015, SEQ ID NO: 72) LGISPMHEHAE (R0023, SEQ ID NO: 73); and RRYTIAAMLSPYS (R0021, SEQ ID NO: 74)] were added and incubated. The plate was washed with [PBS+0.05% Tween20], and the antibody [Ab1] was added and incubated. The plate was washed [PBS+0.05% Tween20] and the anti-mouse-HRP antibody [Jackson ImmunoResearch] was added and incubated. The plate was washed [PBS+0.05% Tween20] a final time and TMB substrate (Biolegend) was added. Phosphoric acid [1M] was added on top to stop the reaction. The plate was read at two absorbance wavelengths (450 nm and 570 nm). No binding to any of the peptides, nor any binding to linear peptides that include the mTTR stabilizing mutations [Phe87Met and Leu110Met] was observed. See FIG. 5.

### Example 11: TTR Sandwich ELISA

Clear 384-well high-binding ELISA plates were coated with the seven murine antibodies from Example 3 and incubated overnight. The plate was washed with PBS-T [PBS+0.05% Tween20] using a robotic liquid handler and then blocked. Untagged TTR (monomer, dimer, stabilized tetramer, or wild-type TTR) was added and incubated. The plate was washed with PBS-T and a HRP-conjugated pan-TTR polyclonal antibody (ImmunoReagents-HRP) was added and incubated. The plate was washed with PBS-T a final time and TMB substrate (Biolegend) was added. Phosphoric acid [1M] was added on top to stop the reaction. The plate was read at two absorbance wavelengths (450 nm and 570 nm). The EC₅₀'s were determined and are included in Table 11. All mAbs showed that they were monomer specific. See FIGS. 3A-C and 4A-C.

TABLE 11
mTTR Binding EC₅₀'s

| Antibody | EC₅₀ (nM) |
|---|---|
| 1 | 13.58 |
| 2 | 98.90 |
| 3 | 128.5 |
| 4 | 11.41 [estimated] |
| 5 | 14.18 [estimated] |
| 6 | 12.66 [estimated] |
| 7 | 43.22 [estimated] |

### Example 12: Detection of TTR Mutants Using Anti-TTR Monomer Antibodies

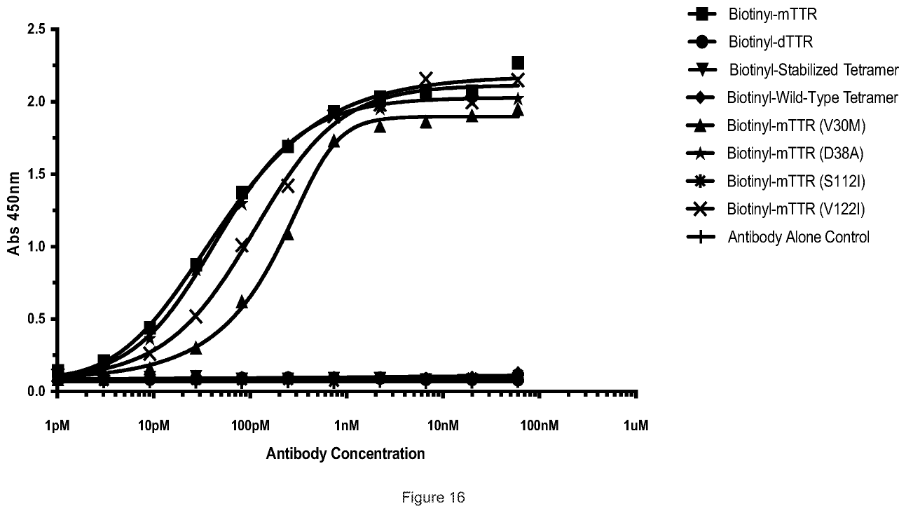

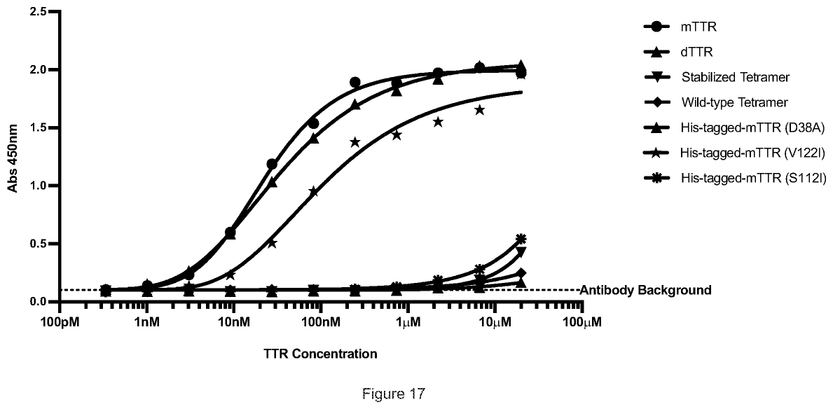

The binding of an antibody of the disclosure, Ab1, to various disease relevant mutants was determined using either the Sandwich ELISA or the Indirect ELISA as described in Examples 11 and 4, respectively. The EC₅₀s were determined and are included in Tables 12-13. See FIGS. 16 and 17.

TABLE 12
Sandwich ELISA

| Antigen | EC₅₀ (nM) |
|---|---|
| Monomer TTR | 22.04 |
| Dimer TTR | No binding |
| Stabilized Tetramer TTR | Slight binding at high end |
| Wild-Type Tetramer TTR | Slight binding at high end |
| His-tagged-Monomer TTR | 31.94 |
| (D38A F87M, L110M) | |
| His-tagged-Monomer | Slight binding at high end |
| TTR (F87M L110M S1121) | |
| His-tagged-Monomer TTR | 100.3 |
| (F87M L110M V1221) | |

TABLE 13
Indirect ELISA

| Antigen | EC₅₀ (nM) |
|---|---|
| Biotinyl-Monomer | 0.048 |
| Biotinyl-Dimer | No binding |
| Biotinyl-Stabilized Tetramer | No binding |
| Biotinyl-Wild-Type Tetramer | No binding |
| Biotinyl-Monomer TTR (V30M F87M L110M) | 0.198 |
| Biotinyl-Monomer TTR (D38A F87M, L110M) | 0.046 |
| Biotinyl-Monomer TTR (F87M L110M S112I) | No binding |
| Biotinyl-Monomer TTR (F87M L110M V122I) | 0.107 |

### Example 13: Dot Blot Assay Indicates Murine and Humanized Anti-TTR Monomer Antibodies Bind TTR Monomer, but not TTR Aggregates

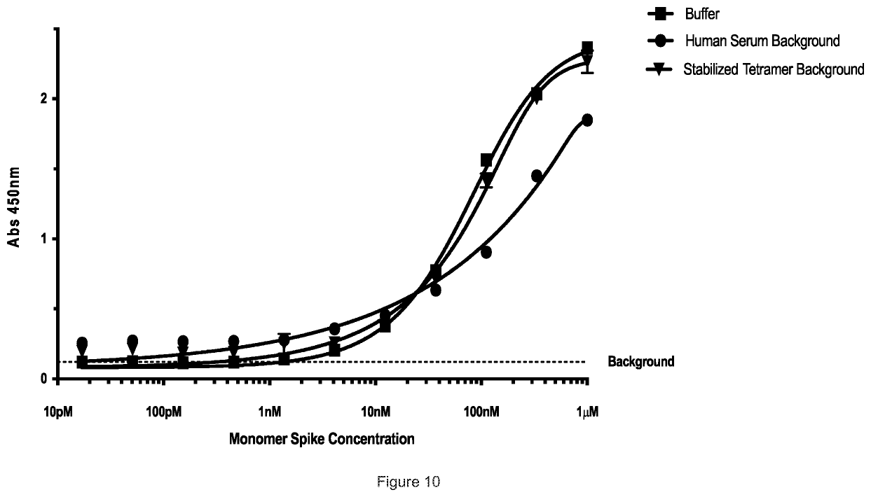

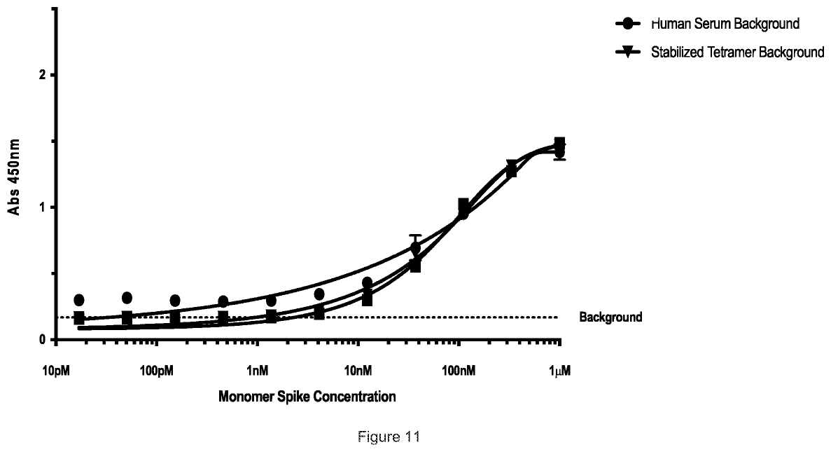

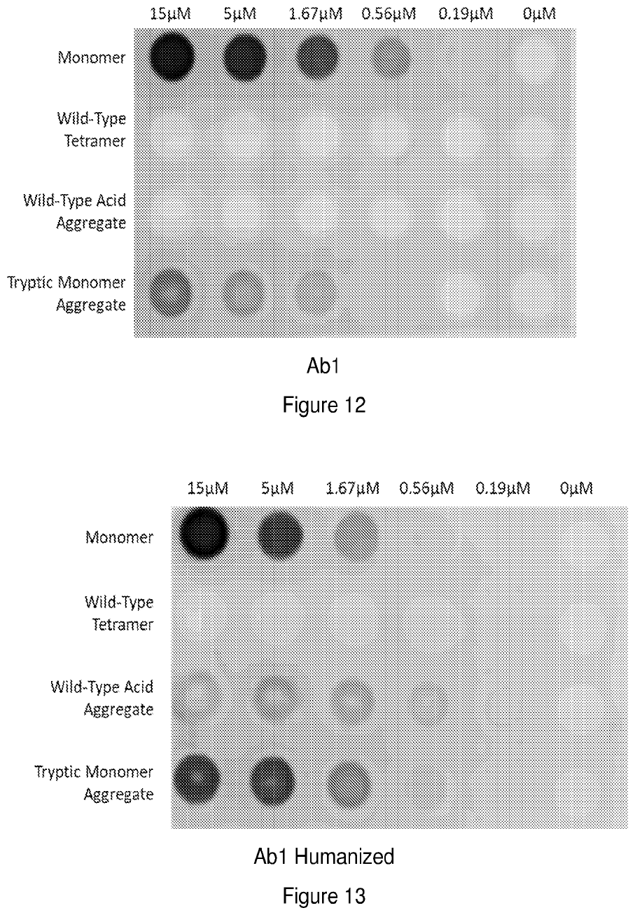

Different forms of TTR (monomeric and wild-type tetrameric TTR) and TTR aggregates (monomer acid, WT-acid, and monomer-tryptic) were bound to nitrocellulose membranes. The membrane was washed to remove any unbound TTR and then the membrane was blocked. Afterwards, the anti-TTR monomer antibodies were added and incubated overnight. The next day, the membrane was washed and an anti-mouse-HRP-conjugated antibody or an anti-human-HRP-conjugated antibody was added and incubated [at 4° C.]. The membrane was washed again and a chemiluminescent substrate [SuperSignal West Pico PLUS Chemiluminescent Substrate] was added for detection. For a loading control of total TTR, the same membranes were washed, stripped of antibody, washed, blocked, and incubated (overnight) with a pan-TTR antibody (DAKO A0002). The membrane was washed again and an anti-rabbit-HRP-conjugated antibody [Jackson ImmunoResearch] was added and incubated at room temperature. The membrane was washed again and a chemiluminescent substrate [SuperSignal West Pico PLUS Chemiluminescent Substrate] was added for detection. Ab1 and humanized Ab1 were analyzed with the dot blot assay to measure specificity for monomer over WT-TTR. The results indicate Ab1 does not bind aggregated TTR. However, humanized Ab1 does have minimal binding to aggregates generated by incubating WT-TTR in mildly acidic conditions. See FIGS. 12-13.

### Example 14: Detection of TTR Monomer in ATTR Patient Plasma Using Anti-TTR Monomer Antibodies

The Simoa™ assay platform (Quanterix) was used for detection of TTR by the Abs of the disclosure. The Simoa™ platform uses a 3-step protocol with Helper Plex/50 uL RGP for HD-X (25 uL RGP for HD-1). The untagged monomeric Phe78Met Leu110Met TTR form, see Example 2, was used as the calibrator; Ab1 was used as the capture reagent. Ab1 (0.2 mg/mL) was conjugated to beads using standard protocol using EDC (0.3 mg/mL, at 4° C.). The Ab beads were diluted to 5×10⁸ beads/mL in Homebrew Bead Diluent. Helper beads (15×10⁶ beads/mL) were added to the reagent. Rabbit pan-TTR pAb antibody (DAKO, A0002) was used as the detector reagent. The detector reagent was conjugated to the beads at a 40× Biotinylation ratio which was diluted ×10 in Simoa Diluent 1 prior to final dilution. The final concentration of detector reagent used was 0.4 ug/mL in Surmodics SM01 Diluent. Streptavidin beta-galactosidase (SBG) was diluted to 300 pM in Simoa™ SBG Diluent. After calibration using the calibrator, samples were analyzed using MRD in Surmodics SM01 diluent. The final volume of diluted sample was run in duplicate is 250 μL in 1 well. The estimated limit of detection of TTR monomer in buffer was 163 pg/ml (11.7 nM).

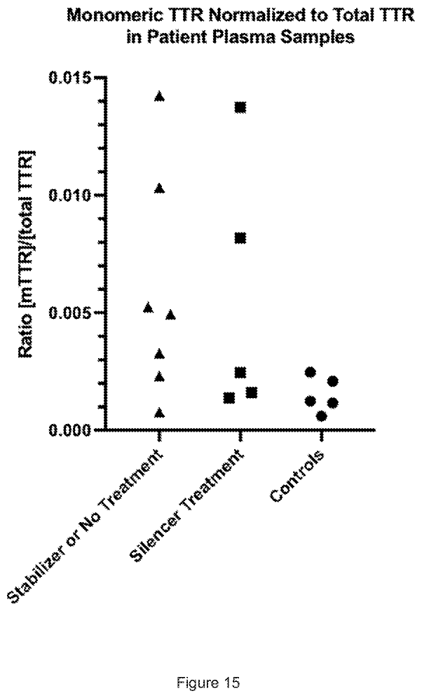

Blood samples were taken from two cohorts of patients with and without ATTR. Monomeric TTR samples were quantitated using the protocol described above. The amounts of monomer in the samples were measured and are shown in FIG. 15.

### Example 15: Detection of TTR Monomer Using Pairs of Anti-TTR Monomer Antibodies

Use of the anti-TTR monomer antibodies in combination, such as one for capture and another for detection, in various aspects, are effective as diagnostics. Such use is carried out using a protocol similar to that described in Example 14, but using combinations of anti-TTR monomer antibodies Ab3 and Ab5 as the capture antibody and the detection antibody. Capture with mAb 5 and detection with mAb 3 showed almost complete overlap between monomer spiked into buffer alone samples and those samples containing high concentrations of tetramer (20 μM recombinant T119M TTR or 100% human serum). See FIGS. 10-11.

Unless otherwise defined, all technical and scientific terms used herein have the same meaning as commonly understood by one of ordinary skill in the art to which this disclosure pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, prevail. In addition, the materials, methods, and examples are illustrative only and not intended to be limiting.

The details of one or more embodiments of the disclosure are also set forth in the accompanying drawings. Other features, objects, and advantages of the disclosure will be apparent from the description and drawings, and from the claims.

One skilled in the art will recognize many methods and materials similar or equivalent to those described herein, which could be used in the practice of the disclosure. Indeed, the disclosure is in no way limited to the methods and materials described.

While the disclosure has been described in terms of specific embodiments, it is understood that variations and modifications will occur to those skilled in the art. Accordingly, only such limitations as appear in the claims should be placed on the disclosure.

All documents referred to in this application are hereby incorporated by reference in their entirety.

Exemplary Embodiments contemplated herein include the following:
1. An isolated antibody that binds to human transthyretin (TTR) wherein the antibody:
   (a) binds to human TTR with an EC₅₀ of less than about 200 nM as determined in a sandwich ELISA; and/or
   (b) binds to human TTR with a K_{D} of less than about 20 nM as determined in a biolayer interferometry assay.
2. The antibody of Embodiment 1 wherein the human TTR is in a monomeric form.
3. The antibody of Embodiment 2 wherein the antibody binds monomeric TTR with an EC₅₀ of less than about 20 nM as determined in an indirect ELISA.
4. The antibody of any one of Embodiments 1-3 wherein the antibody prevents aggregation of TTR.
5. The antibody of any one of Embodiments 1-4 wherein the antibody binds a folding intermediate of TTR.
6. The antibody of Embodiment 1 comprising:
a heavy chain variable domain (VH) comprising
   (a) a CDR-H1 comprising the amino acid sequence of any one of SEQ ID NOs: 2-8;
   (b) a CDR-H2 comprising the amino acid sequence of any one of SEQ ID NOs: 9-15; and
   (c) a CDR-H3 comprising the amino acid sequence of any one of SEQ ID NOs: 16-22; and
a light chain variable domain (VL) comprising
   (d) a CDR-L1 comprising the amino acid sequence of any one of SEQ ID NOs: 23-29;
   (e) a CDR-L2 comprising the amino acid sequence of any one of SEQ ID NOs: 30-36; and
   (f) a CDR-L3 comprising the amino acid sequence of any one of SEQ ID NOs: 37-43.
7. An antibody that binds to human transthyretin (TTR), the antibody comprising a heavy chain variable domain (VH) comprising
   (a) a CDR-H1 comprising the amino acid sequence of any one of SEQ ID NOs: 2-8;
   (b) a CDR-H2 comprising the amino acid sequence of any one of SEQ ID NOs: 9-15; and
   (c) a CDR-H3 comprising the amino acid sequence of any one of SEQ ID NOs: 16-22; and
a light chain variable domain (VL) comprising
   (d) a CDR-L1 comprising the amino acid sequence of any one of SEQ ID NOs: 23-29;
   (e) a CDR-L2 comprising the amino acid sequence of any one of SEQ ID NOs: 30-36; and
   (f) a CDR-L3 comprising the amino acid sequence of any one of SEQ ID NOs: 37-43.
8. The antibody of any one of Embodiments 1-4 comprising:
   (a) a CDR-H1 comprising the amino acid sequence of SEQ ID NO: 2;
   (b) a CDR-H2 comprising the amino acid sequence of SEQ ID NO: 9; and
   (c) a CDR-H3 comprising the amino acid sequence of SEQ ID NO: 16; and
a light chain variable domain (VL) comprising
   (d) a CDR-L2 comprising the amino acid sequence of SEQ ID NO: 23;
   (e) a CDR-L2 comprising the amino acid sequence of SEQ ID NO: 30; and
   (f) a CDR-L3 comprising the amino acid sequence of SEQ ID NO: 37.
9. The antibody of any one of Embodiments 1-4 comprising:
a heavy chain variable domain (VH) comprising
   (a) a CDR-H1 comprising the amino acid sequence of SEQ ID NO: 3;
   (b) a CDR-H2 comprising the amino acid sequence of SEQ ID NO: 10; and
   (c) a CDR-H3 comprising the amino acid sequence of SEQ ID NO: 17; and
a light chain variable domain (VL) comprising
   (d) a CDR-L2 comprising the amino acid sequence of SEQ ID NO: 24;
   (e) a CDR-L2 comprising the amino acid sequence of SEQ ID NO: 31; and
   (f) a CDR-L3 comprising the amino acid sequence of SEQ ID NO: 38.
10. The antibody of any one of Embodiments 1-4 comprising:
a heavy chain variable domain (VH) comprising
   (a) a CDR-H1 comprising the amino acid sequence of SEQ ID NO: 4;
   (b) a CDR-H2 comprising the amino acid sequence of SEQ ID NO: 11; and
   (c) a CDR-H3 comprising the amino acid sequence of SEQ ID NO: 18; and
a light chain variable domain (VL) comprising
   (d) a CDR-L2 comprising the amino acid sequence of SEQ ID NO: 25;
   (e) a CDR-L2 comprising the amino acid sequence of SEQ ID NO: 32; and
   (f) a CDR-L3 comprising the amino acid sequence of SEQ ID NO: 39.
11. The antibody of any one of Embodiments 1-4 comprising:
a heavy chain variable domain (VH) comprising
   (a) a CDR-H1 comprising the amino acid sequence of SEQ ID NO: 5;
   (b) a CDR-H2 comprising the amino acid sequence of SEQ ID NO: 12; and
   (c) a CDR-H3 comprising the amino acid sequence of SEQ ID NO: 19; and
a light chain variable domain (VL) comprising
   (d) a CDR-L2 comprising the amino acid sequence of SEQ ID NO: 26;
   (e) a CDR-L2 comprising the amino acid sequence of SEQ ID NO: 33; and
   (f) a CDR-L3 comprising the amino acid sequence of SEQ ID NO: 40.
12. The antibody of any one of Embodiments 1-4 comprising:
a heavy chain variable domain (VH) comprising
   (a) a CDR-H1 comprising the amino acid sequence of SEQ ID NO: 6;
   (b) a CDR-H2 comprising the amino acid sequence of SEQ ID NO: 13; and
   (c) a CDR-H3 comprising the amino acid sequence of SEQ ID NO: 20; and
a light chain variable domain (VL) comprising
   (d) a CDR-L2 comprising the amino acid sequence of SEQ ID NO: 27;
   (e) a CDR-L2 comprising the amino acid sequence of SEQ ID NO: 34; and
   (f) a CDR-L3 comprising the amino acid sequence of SEQ ID NO: 41.
13. The antibody of any one of Embodiments 1-4 comprising:
a heavy chain variable domain (VH) comprising
   (a) a CDR-H1 comprising the amino acid sequence of SEQ ID NO: 7;
   (b) a CDR-H2 comprising the amino acid sequence of SEQ ID NO: 14; and
   (c) a CDR-H3 comprising the amino acid sequence of SEQ ID NO: 21; and
a light chain variable domain (VL) comprising
   (d) a CDR-L2 comprising the amino acid sequence of SEQ ID NO: 28;
   (e) a CDR-L2 comprising the amino acid sequence of SEQ ID NO: 35; and
   (f) a CDR-L3 comprising the amino acid sequence of SEQ ID NO: 42.
14. The antibody of any one of Embodiments 1-4 comprising:
a heavy chain variable domain (VH) comprising
   (a) a CDR-H1 comprising the amino acid sequence of SEQ ID NO: 8;
   (b) a CDR-H2 comprising the amino acid sequence of SEQ ID NO: 15;
   (c) a CDR-H3 comprising the amino acid sequence of SEQ ID NO: 22; and
a light chain variable domain (VL) comprising
   (d) a CDR-L2 comprising the amino acid sequence of SEQ ID NO: 29;
   (e) a CDR-L2 comprising the amino acid sequence of SEQ ID NO: 36; and
   (f) a CDR-L3 comprising the amino acid sequence of SEQ ID NO: 43.
15. The antibody of any one of Embodiments 1-14 which is a monoclonal antibody.
16. The antibody of any one of Embodiments 1-15 which is a humanized or chimeric antibody.
17. The antibody of Embodiment 16 wherein the VH domain comprises a sequence comprising at least 85% sequence identity to the sequence of any one of SEQ ID NOs: 46-49.
18. The antibody of Embodiment 16 wherein the VH domain comprises the sequence of any one of SEQ ID NOs: 46-49.
19. The antibody of Embodiment 16 wherein the VH domain comprises a sequence comprising at least 85% sequence identity to the sequence of any one of SEQ ID NOs: 55-60.
20. The antibody of Embodiment 16 wherein the VH domain comprises the sequence of any one of SEQ ID NOs: 55-60.
21. The antibody of Embodiment 16 wherein the VL domain comprises at least 85% sequence identity to the sequence of any one of SEQ ID NOs: 51-53.
22. The antibody of Embodiment 16 wherein the VL domain comprises the sequence of any one of SEQ ID NOs: 51-53.
23. The antibody of Embodiment 16 wherein the VL domain comprises a sequence comprising at least 85% sequence identity to the sequence of any one of SEQ ID NOs: 62-64.
24. The antibody of Embodiment 16 wherein the VL domain comprises the sequence of any one of SEQ ID NOs: 62-64.
25. The antibody of any one of Embodiments 1-24 which is an antibody fragment that binds to a monomeric form or a folding intermediate form of human TTR.
26. The antibody of any one of Embodiments 1-25 that does not bind to a peptide having an amino acid sequence selected from any one of GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69), LSPYSY (SEQ ID NO: 70), SYSTTAVV (SEQ ID NO: 71), YTIAALSPYSYSTTAVV (SEQ ID NO: 72), LGISPMHEHAE (SEQ ID NO: 73) and RRYTIAAMLSPYS (SEQ ID NO: 74).
27. The antibody of Embodiment 26 that binds to a conformational epitope.
28. The antibody of any of Embodiments 1-27 which is an IgG4 antibody.
29. The antibody of any one of Embodiments 1-28 wherein the antibody binds human TTR with an EC₅₀ of less than about 150 nM, or less than about 100 nM, or less than about 50 nM, or less than about 20 nM, as determined in a sandwich ELISA.
30. The antibody of any one of Embodiments 1-29, wherein the antibody binds human TTR with an EC₅₀ of less than about 20 nM, or less than about 5 nM, or less than about 2 nM, or less than about 1 nM, as determined using an Indirect ELISA.
31. An antibody that specifically binds human TTR and that competes for binding to human TTR with the antibody of any one of Embodiments 1-30.
32. An isolated nucleic acid comprising a nucleotide sequence encoding the antibody of any of Embodiments 1-31.
33. An isolated host cell comprising the nucleic acid of Embodiment 32.
34. An isolated host cell that express the antibody of any one of Embodiments 1-31.
35. A method of producing an antibody that binds to human TTR comprising culturing the host cell of Embodiment 33 or Embodiment 34 under conditions suitable for the expression of the antibody.
36. The method of Embodiment 35, further comprising recovering the antibody from the host cell or host cell culture.
37. An antibody produced by the method of Embodiment 35 or 36.
38. A composition comprising the antibody of any of Embodiments 1-31 and 37.
39. The antibody of any one of Embodiments 1-31 and 37, or the composition of Embodiment 38 for use in treating, preventing, or diagnosing a disease associated with associated with TTR aggregation.
40. The antibody of any one of Embodiments 1-31 and 37, or the composition of Embodiment 38 for use in treating TTR amyloidosis.
41. The antibody of Embodiment 40 wherein the TTR amyloidosis is transthyretin amyloidosis (ATTR) cardiomyopathy (ATTR-CM) or transthyretin amyloidosis (ATTR) polyneuropathy (ATTR-PN).
42. The antibody of any one of Embodiments 1-31 and 37, or the composition of Embodiment 38 for use in treating peripheral TTR amyloidosis, ocular amyloid angiopathy or cerebral amyloid angiopathy.
43. The antibody of any one of Embodiments 1-31 and 37, or the composition of Embodiment 38 for use in treating a disease selected from Familial Amyloid Polyneuropathy (AP), Familial Amyloid Cardiomyopathy (FAC), ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia.
44. A method of treating a subject having a disease associated with TTR amyloidosis, the method comprising administering to the subject an effective amount of the antibody of any one of Embodiments 1-31 and 37, or the composition of Embodiment 38.
45. The method of Embodiment 44 wherein the disease is TTR amyloidosis.
46. A method of treating a subject having a disease selected from Familial Amyloid Polyneuropathy (AP), Familial Amyloid Cardiomyopathy (FAC), ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia, the method comprising administering to the subject an effective amount of the antibody of any one of Embodiments 1-31 and 37, or the composition of Embodiment 38.
47. The antibody of any one of Embodiments 1-31 and 37 further comprising a detectable label.
48. A method of measuring monomeric transthyretin (TTR) in a sample, the method comprising contacting the sample with the antibody of any one of Embodiments 1-31, 37, and 47, and measuring the amount of antibody bound to TTR in the sample.
49. The method according to Embodiment 48, wherein the measuring comprises contacting the sample with a secondary antibody and measuring a complex formed thereby. 50. The method according to Embodiment 49 that comprises using TTR monomer antibodies Ab3 and Ab5, with one of said antibodies used as a capture antibody and one of said antibodies used as a detection antibody.
51. A method of diagnosing a disease associated with transthyretin (TTR) amyloidosis, or monitoring the treatment of the disease with an anti-TTR therapy comprising assaying the level of monomeric TTR in a sample of a body fluid from a subject, wherein the presence or an elevated level of monomeric TTR in the sample of the subject compared to a control indicates the presence of a disease associated with TTR amyloidosis.
52. The method of Embodiment 51 wherein the monomeric TTR binds to the antibody of any one of Embodiments 1-31 and 37.
53. A method comprising:
   contacting a sample from a subject with a sample of a bodily fluid from a subject having or suspected of having transthyretin (TTR) amyloidosis; and
   detecting or measuring monomeric TTR in the sample.
54. The method according to Embodiment 53, wherein the contacting step uses a contacting antibody selected from TTR monomer antibodies Ab3 and Ab5.
55. The method according to Embodiment 54, wherein the detecting or measuring step comprises contacting a complex formed by the contacting antibody and the TTR with a detecting antibody selected from TTR monomer antibodies Ab3 and Ab5, wherein the contacting antibody is different from the detecting antibody.
56. The method according to any one of Embodiments 53-55, further comprising initiating anti-TTR therapy based on detecting or measuring the monomeric TTR in the sample.
57. The method according to any one of Embodiments 53-55, further comprising adjusting the dose of an anti-TTR therapy based on the measurement of the monomeric TTR in the sample.
58. The method according to Embodiment 56 or 57, wherein the anti-TTR therapy comprises administering to the subject an antibody according to any one of Embodiments 1-31 and 37 or a composition according to Embodiment 38.
59. A composition comprising the antibody of any one of Embodiments 1-31 and 37 wherein the composition (i) is a pharmaceutical composition and further comprises a pharmaceutically acceptable carrier, or (ii) a diagnostic composition.
60. The composition of Embodiment 59 wherein the diagnostic composition further comprises reagents conventionally used in immuno-based or antibody-based diagnostic methods.
61. The composition of Embodiment 59 wherein the diagnostic method measures a TTR monomeric form.
62. The composition of Embodiment 59 wherein the diagnostic method uses the Quanterix platform.
63. The composition of Embodiment 59 wherein the diagnostic method is a Western gel.
64. An isolated antibody which recognizes a conformational epitope on monomeric TTR prepared from F87M L110M TTR that is not present on tetrameric TTR and physiological, cellular full-length TTR.
65. An isolated antibody which recognizes a TTR mutation selected from any of V30M, D38A and V1221, wherein, the antibody specifically binds TTR comprising the amino acid sequence set forth in any of SEQ ID NOs: 145-147.
66. The antibody of Embodiment 65 wherein the TTR mutation is D38A and/or V1221, wherein the antibody specifically binds TTR comprising the amino acid sequence set forth in SEQ ID NO: 146 and/or 147.
67. The antibody of Embodiment 16 wherein the VH domain comprises at least 85% sequence identity with any of the sequences set forth in FIGS. 6A-6B, 7, 14, and 18-21 (SEQ ID NOs: 81-142).
68. The antibody of Embodiment 16 wherein the VL domain comprises at least 85% sequence identity with any of the sequences set forth in FIGS. 6A-6B, 7, 14, and 18-21 (SEQ ID NOs: 81-142).
69. The antibody of any one of Embodiments 1-31 and 37, wherein the antibody specifically binds to TTR comprising the amino acid sequence set forth in any one of SEQ ID NOs: 80 and 143-147 and 149-152.
70. The antibody of Embodiment 69, wherein the antibody specifically binds to TTR comprising the amino acid sequence set forth in SEQ ID NO: 80.
71. A method of producing a cell that produces an antibody that specifically binds to a monomeric form of human transthyretin (TTR), the method comprising:
   (a) immunizing a non-human animal with a monomeric form of TTR;
   (b) screening blood or tissue from the animal for an antibody which specifically binds to the monomeric form of human TTR but does not bind to wild-type tetrameric TTR or to a dimeric or tetrameric form of TTR; and
   (c) identifying and isolating a cell from the animal that produces the antibody that specifically binds to the monomeric form of human TTR but does not bind to wild-type tetrameric TTR or to a dimeric or tetrameric form of TTR.
72. The method of Embodiment 71, wherein the antibody-producing cell is recovered by removal of spleen tissue, lymph nodes or bone marrow of the animal.
73. The method of any one of Embodiments 71-72, wherein the antibody-producing cell is a B cell, a T cell, or a stem cell.
74. The method of any one of Embodiments 71-73, wherein the animal is a mouse, a rat, a guinea pig, or a rabbit.
75. The method of any one of Embodiments 71-74, further comprising immortalizing the isolated cell.
76. The method of Embodiment 75, comprising producing a hybridoma cell by somatic fusion of the cell to a myeloma cell.
77. The method of any one of Embodiments 71-76, further comprising culturing the antibody-producing cell.
78. The method of any one of Embodiments 71-77, wherein the antibody specifically binds to TTR comprising the amino acid sequence set forth in any one of SEQ ID NOs: 80, 143, and 144.
79. The method of any one of Embodiments 71-78, wherein the antibody does not bind to a peptide having an amino acid sequence consisting of a sequence selected from any one of GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69), LSPYSY (SEQ ID NO: 70), SYSTTAVV (SEQ ID NO: 71), YTIAALLSPYSYSTTAVV (SEQ ID NO: 72), LGISPMHEHAE (SEQ ID NO: 73) and RRYTIAAMLSPYS (SEQ ID NO: 74).
80. The method of any one of Embodiments 71-79, wherein the monomeric form of human TTR used to immunize the animal is any one of
   TTR F87M L110M comprising the amino acid sequence set forth in SEQ ID NO: 80;
   TTR F87M comprising the amino acid sequence set forth in SEQ ID NO: 143; and
   TTR L110M comprising the amino acid sequence set forth in SEQ ID NO: 144.
81. An antibody produced by the method of any one of Embodiments 71-80.
82. A method for producing a monoclonal antibody that specifically binds to a monomeric form of human transthyretin (TTR), said method comprising:
   (a) introducing into a non-human animal at least one of any one of
   TTR F87M L110M comprising the amino acid sequence set forth in SEQ ID NO: 80;
   TTR F87M comprising the amino acid sequence set forth in SEQ ID NO: 143; and
   TTR L110M comprising the amino acid sequence set forth in SEQ ID NO: 144;
   (b) recovering antibody-producing cells from the animal and rendering these cells into a single cell suspension;
   (c) generating an immortalized cell line from the single cell suspension;
   (d) screening the supernatant of the immortalized cell line for the presence of an antibody having binding specifically for a monomeric form of human TTR; and
   (e) selecting, as the monoclonal antibody, an antibody that specifically binds to the monomeric form of human TTR.
83. The method of Embodiment 82, wherein said animal is a mouse, a rat, a guinea pig or a rabbit.
84. The method of Embodiment 82 or 83, wherein the antibody-producing cells are B cells, T cells or stem cells.
85. The method of any one of Embodiments 82-84, wherein the antibody-producing cells are recovered by removal of spleen tissue, lymph nodes or bone marrow of the animal.
86. The method of any one of Embodiments 82-85, wherein the immortalized cell line is a hybridoma cell line produced by somatic fusion of the cells in the single cell suspension to myeloma cells.
87. An antibody produced by the method of any one of Embodiments 82-86.
88. A method comprising:
   (a) assaying a library of molecules for a molecule peptide or polypeptide that:
      (i) binds one or more monomeric TTR peptides having amino acid an amino acid sequence selected from SEQ ID NOs: 80 and 143-147 and 149-152; and
      (ii) exhibits substantially no binding affinity for a tetrameric human TTR protein having the amino acid sequence of SEQ ID NO: 153.
   (b) identifying a molecule that exhibits the binding properties of (i) and (ii); and
   (c) isolating the molecule identified in (b), or isolating a cell that expresses said molecules.
89. The method according to Embodiment 88, further comprising assaying to identify a molecule that exhibits the binding properties of (i) and (ii) and that (iii) fails to bind to a peptide having an amino acid sequence consisting of SEQ ID NO: 65, 66, 67, 68, 69, 70, 71, 72, 73, or 74.
90. The method according to Embodiment 88 or 89, wherein molecules comprise antibodies or antigen-binding fragments of antibodies.
91. The method according to Embodiment 90, wherein the isolating step comprises isolating a cell that produces an antibody that exhibits the binding properties of (i) and (ii) and optionally (iii).
92. The method according to any one of Embodiments 88-91 that comprises a step, prior to the assaying step, of immunizing a non-human mammal with the one or more monomeric TTR peptides, and the assaying comprises screening antibody molecules produced by the mammal.
93. The method according to any one of Embodiments 88-92, further comprising:
   determining amino acid sequence of at least the complementarity-determining regions (CDR) of the antibody;
   generating humanized antibody sequences that includes said amino acid sequences of said CDRs; and
   synthesizing nucleic acid that comprises nucleotide sequences that encode the humanized antibody.
94. The method according to Embodiment 93, further comprising transfecting a cell with a nucleic acid that encodes the humanized antibody sequence, or with a nucleic acid that encodes an antigen-binding fragment of the humanized antibody sequence.
95. The method of Embodiment 94, further comprising culturing the cell under conditions to express the antibody or the antigen-binding fragment.
96. The method according to Embodiment 88 or 89, further comprising synthesizing copies of the molecule identified as having the binding properties of (i) and (ii) and, optionally (iii).
97. An isolated molecule produced by the process of Embodiment 95 or 96.
98. A pharmaceutical composition comprising the isolated molecule of Embodiment 97 and a pharmaceutically acceptable carrier.

TABLE 14
Sequence Table.

| SEQ ID | | |
|---|---|---|
| NO: | Descriptor | SEQUENCE |
| 1 | hTTR | GPTGTGESKCPLMVKVLDAVRGSPAINVAVHVFRKAADDTWEPFASGK |
| | | TSESGELHGLTTEEEFVEGIYKVEIDTKSYWKALGISPFHEHAEVVFTAND |
| | | SGPRRYTIAALLSPYSYSTTAVVTNPKE |
| 2 | CDR-H1 | GYTFTSYW |
| 3 | CDR-H1 | GFTFSNYW |
| 4 | CDR-H1 | GYTFTDYY |
| 5 | CDR-H1 | GYTFTTYG |
| 6 | CDR-H1 | GFNIKDYY |
| 7 | CDR-H1 | GYTFTGYW |
| 8 | CDR-H1 | GFNIKDYY |
| 9 | CDR-H2 | IDPSDSET |
| 10 | CDR-H2 | IRLKSDNYAT |
| 11 | CDR-H2 | INPYNGGT |
| 12 | CDR-H2 | INTYSGVP |
| 13 | CDR-H2 | IDPEDGET |
| 14 | CDR-H2 | ILPGSGST |
| 15 | CDR-H2 | IDPEDGET |
| 16 | CDR-H3 | ARGRNYWYFDV |
| 17 | CDR-H3 | TGVFTY |
| 18 | CDR-H3 | ARDLTGTGGAMDY |
| 19 | CDR-H3 | AFYYDYQEWFAY |
| 20 | CDR-H3 | ALLWLRRGRGY |
| 21 | CDR-H3 | ARLRGSSYPWYFDY |
| 22 | CDR-H3 | ARGKTPFAY |
| 23 | CDR-L1 | ESVDNYGISF |
| 24 | CDR-L1 | ESVDSYGNSF |
| 25 | CDR-L1 | SSVSY |
| 26 | CDR-L1 | ENIYSN |
| 27 | CDR-L1 | QEISGY |
| 28 | CDR-L1 | GYTFTGYW |
| 29 | CDR-L1 | ENIYSN |
| 30 | CDR-L2 | AAS |
| 31 | CDR-L2 | LAS |
| 32 | CDR-L2 | ATS |
| 33 | CDR-L2 | AAT |
| 34 | CDR-L2 | AAS |
| 35 | CDR-L2 | AAT |
| 36 | CDR-L2 | AAS |
| 37 | CDR-L3 | QQSKEVPWT |
| 38 | CDR-L3 | QQNNEDPMYT |
| 39 | CDR-L3 | QQWSSNPLT |
| 40 | CDR-L3 | QHFWGTPR |
| 41 | CDR-L3 | LQYASYPPT |
| 42 | CDR-L3 | QHFWGTPYT |
| 43 | CDR-L3 | LQYDSYPFT |
| 44 | Formula 1 | Gly-X¹-X²-X³-X⁴-X⁵-Tyr-X⁶ |
| | CDR-H1 | Wherein X1 is Tyr or Phe; |
| | | X2 is Thr or Asn; |
| | | X3 is Phe or lle; |
| | | X4 is Thr, Ser or Lys; |
| | | X5 is selected from Asp, Asn, Thr, Ser and Gly; and |
| | | X6 is Trp Tyr or Gly. |
| 45 | Anti-mTTR | QVQLQQPGAELVRPGSSVKLSCKASGYTFTSYWMHWVKQRPIQGLEWI |
| | VH (Ab1) | GNIDPSDSETHYNQKFKDKATLTVDKSSSTAYMQLSSLTSEDSAVYYCA |
| | | RGRNYWYFDVWGTGTTVTVSS |
| 46 | Anti-mTTR | QVQLVQSGAEVKKPGASVKVSCKASGYTFTSYWMHWVRQAPGQGLE |
| | VH | WMGNIDPSDSETHYAQKFQGRVTMTRDTSTSTVYMELSSLRSEDTAVY |
| | | YCARGRNYWYFDVWGTGTMVTVSS |
| 47 | Anti-mTTR | QVQLVQSGAEVKKPGASVKVSCKASGYTFTSYWMHWVRQAPGQGLE |
| | VH | WMGNIDPSDSETHYAQKFQGRVTMTVDTSTSTVYMELSSLRSEDTAVY |
| | | YCARGRNYWYFDVWGTGTMVTVSS |
| 48 | Anti-mTTR | QVQLVQSGAEVKKPGASVKVSCKASGYTFTSYWMHWVRQAPGQGLE |
| | VH | WMGNIDPSDSETHYAQKFQGRVTMTVDKSTSTVYMELSSLRSEDTAVY |
| | | YCARGRNYWYFDVWGTGTMVTVSS |
| 49 | Anti-mTTR | QVQLVQSGAEVKKPGASVKVSCKASGYTFTSYWMHWVRQAPGQGLE |
| | VH | WIGNIDPSDSETHYAQKFQGRATLTVDKSTSTVYMELSSLRSEDTAVYY |
| | humanized | CARGRNYWYFDVWGTGTMVTVSS |
| | Ab1 | |
| 50 | Anti-mTTR | DIVLTQSPASLAVSLGQRATISCRASESVDNYGISFMNWFQQKPGQPPK |
| | VL (Ab1) | LLIYAASNQGSGVPARFSGSGSGTDFSLNIHPMEEDDTAMYFCQQSKEV |
| | | PWTFGGGTKLEIK |
| 51 | Anti-mTTR | DIQMTQSPSSLSASVGDRVTITCRASESVDNYGISFMNWYQQKPGKAPK |
| | VL | LLIYAASNQGSGVPSRFSGSGSGTDFTLTISSLQPEDFATYYCQQSKEVP |
| | | WTFGGGTKLEIK |
| 52 | Anti-mTTR | DIQMTQSPSSLSASVGDRVTITCRASESVDNYGISFMNWFQQKPGKAPK |
| | VL | LLIYAASNQGSGVPSRFSGSGSGTDFTLTISSLQPEDFATYFCQQSKEVP |
| | | WTFGGGTKLEIK |
| 53 | Anti-mTTR | DIQMTQSPSSLSASVGDRVTITCRASESVDNYGISFMNWFQQKPGKPPK |
| | VL | LLIYAASNQGSGVPSRFSGSGSGTDFTLTISSLQPEDFATYYCQQSKEVP |
| | humanized | WTFGGGTKLEIK |
| | Ab1 | |
| 54 | Anti-mTTR | EVKLEQSGGGLVQPGGSMKLSCVASGYTFSNYWMNWVRQSPEKGLE |
| | VH (Ab2) | WVAQIRLKSDNYATHYAESVKGRFTISRDDSKSSVYLQMNNLRAEDTGI |
| | | YYCTGVFTYWGQGTLVTVSA |
| 55 | Anti-mTTR | EVQLVESGGGLVKPGGSLRLSCAASGYTFSNYWMNWVRQAPGKGLEW |
| | VH | VGQIRLKSDNYATHYAAPVKGRFTISRDDSKNTLYLQMNSLKTEDTAVYY |
| | | CTTVFTYWGQGTLVTVSS |
| 56 | Anti-mTTR | EVQLVESGGGLVKPGGSLRLSCAASGYTFSNYWMNWVRQAPGKGLEW |
| | VH | VGQIRLKSDNYATHYAAPVKGRFTISRDDSKNTLYLQMNSLKTEDTAVYY |
| | | CTGVFTYWGQGTLVTVSS |
| 57 | Anti-mTTR | EVQLVESGGGLVKPGGSLRLSCAASGYTFSNYWMNWVRQAPGKGLEW |
| | VH | VAQIRLKSDNYATHYAAPVKGRFTISRDDSKNTVYLQMNSLKTEDTAVYY |
| | | CTGVFTYWGQGTLVTVSS |
| 58 | Anti-mTTR | EVQLLESGGGLVQPGGSLRLSCAASGYTFSNYWMNWVRQAPGKGLEW |
| | VH | VSQIRLKSDNYATHYADSVKGRFTISRDNSKNTLYLQMNSLRAEDTAVYY |
| | | CAKVFTYWGQGTLVTVSS |
| 59 | Anti-mTTR | EVQLLESGGGLVQPGGSLRLSCAASGYTFSNYWMNWVRQAPGKGLEW |
| | VH | VSQIRLKSDNYATHYADSVKGRFTISRDNSKNTLYLQMNSLRAEDTAVYY |
| | | CTGVFTYWGQGTLVTVSS |
| 60 | Anti-mTTR | EVQLLESGGGLVQPGGSLRLSCAASGYTFSNYWMNWVRQAPGKGLEW |
| | VH | VAQIRLKSDNYATHYADSVKGRFTISRDDSKNTVYLQMNSLRAEDTAVY |
| | humanized | YCTGVFTYWGQGTLVTVSS |
| | Ab2 | |
| 61 | Anti-mTTR | NIVLTQSPASLAVSLGQRATISCRTSESVDSYGNSFMHWYQQKPGQPPK |
| | VL (Ab2) | LLIYLASNLESGVPARFSGSGSRTDFTLTIDPVEADDAATYYCQQNNEDP |
| | | MYTFGGGTKLEIK |
| 62 | Anti-mTTR | DIVMTQSPDSLAVSLGERATINCRTSESVDSYGNSFMHWYQQKPGQPP |
| | VL | KLLIYLASNLESGVPDRFSGSGSGTDFTLTISSLQAEDVAVYYCQQNNED |
| | | PMYTFGGGTKVEIK |
| 63 | Anti-mTTR | DIVMTQSPDSLAVSLGERATINCRTSESVDSYGNSFMHWYQQKPGQPP |
| | VL | KLLIYLASNLESGVPDRFSGSGSRTDFTLTISSLQAEDVAVYYCQQNNED |
| | humanized | PMYTFGGGTKVEIK |
| | Ab2 | |
| 64 | Anti-mTTR | DIVLTQSPDSLAVSLGERATINCRTSESVDSYGNSFMHWYQQKPGQPPK |
| | VL | LLIYLASNLESGVPDRFSGSGSRTDFTLTISSLQAEDVAVYYCQQNNEDP |
| | | MYTFGGGTKVEIK |
| 65 | peptide | GPTGTGESKQPL |
| 66 | peptide | MVKVLDAVRGSPA |
| 67 | peptide | PFHEHAEVVFTA |
| 68 | peptide | EHAEVVFTA |
| 69 | peptide | YTIAALLS |
| 70 | peptide | LSPYSY |
| 71 | peptide | SYSTTAW |
| 72 | peptide | YTIAALLSPYSYSTTAVV |
| 73 | peptide | LGISPMHEHAE |
| 74 | peptide | RRYTIAAMLSPYS |
| 75 | gap | |
| 76 | His_F87M | MGSSHHHHHHSSGLVPAGSHMGPTGTGESKCPLMVKVLDAVRGSPAIN |
| | L110M | VAVHVFRKAADDTWEPFASGKTSESGELHGLTTEEEFVEGIYKVEIDTKS |
| | TTR | YWKALGISPMHEHAEVVFTANDSGPRRYTIAAMLSPYSYSTTAVVTNPK |
| | | E |
| 77 | AA | GLNDIFEAQKIEWHE |
| | insertion | |
| 78 | Avi_mTTR- | MHHHHHHSGMSGLNDIFEAQKIEWHEGENLYFQGMGPTGTGESKCPL |
| | in vivo | MVKVLDAVRGSPAINVAVHVFRKAADDTWEPFASGKTSESGELHGLTTE |
| | biotinylated | EEFVEGIYKVEIDTKSYWKALGISPMHEHAEVVFTANDSGPRRYTIAAML |
| | construct | SPYSYSTTAVVTNPKE |
| 79 | gap | |
| 80 | F87M | GPTGTGESKCPLMVKVLDAVRGSPAINVAVHVFRKAADDTWEPFASGK |
| | L110M | TSESGELHGLTTEEEFVEGIYKVEIDTKSYWKALGISPMHEHAEVVFTAN |
| | TTR | DSGPRRYTIAAMLSPYSYSTTAVVTNPKE |
| 81 | Ab1MC | QVQLQQPGAELVRPGSSVKLSCKASGYTFTSYWMHWVKQRPIQGLEWI |
| | | GNIDPSDSETHYNQKFKDKATLTVDKSSSTAYMQLSSLTSEDSAVYYCA |
| | | RGRNYWYFDVWGTGTTVTVSSASTKGPSVFPLAPCSRSTSESTAALGC |
| | | LVKDYFPEPVTVSWNSGALTSGVHTFPAVLQSSGLYSLSSVVTVPSSSL |
| | | GTKTYTCNVDHKPSNTKVDKRVESKYGPPCPPCPAPEFLGGPSVFLFPP |
| | | KPKDTLMISRTPEVTCVVVDVSQEDPEVQFNWYVDGVEVHNAKTKPRE |
| | | EQFNSTYRVVSVLTVLHQDWLNGKEYKCKVSNKGLPSSIEKTISKAKGQ |
| | | PREPQVYTLPPSQEEMTKNQVSLTCLVKGFYPSDIAVEWESNGQPENN |
| | | YKTTPPVLDSDGSFFLYSRLTVDKSRWQEGNVFSCSVMHEALHNHYTQ |
| | | KSLSLSLGK |
| 82 | Ab2MC | EVKLEQSGGGLVQPGGSMKLSCVASGFTFSNYWMNWVRQSPEKGLE |
| | | WVAQIRLKSDNYATHYAESVKGRFTISRDDSKSSVYLQMNNLRAEDTGI |
| | | YYCTGVFTYWGQGTLVTVSAASTKGPSVFPLAPCSRSTSESTAALGCLV |
| | | KDYFPEPVTVSWNSGALTSGVHTFPAVLQSSGLYSLSSVVTVPSSSLGT |
| | | KTYTCNVDHKPSNTKVDKRVESKYGPPCPPCPAPEFLGGPSVFLFPPKP |
| | | KDTLMISRTPEVTCVVVDVSQEDPEVQFNWYVDGVEVHNAKTKPREEQ |
| | | FNSTYRVVSVLTVLHQDWLNGKEYKCKVSNKGLPSSIEKTISKAKGQPR |
| | | EPQVYTLPPSQEEMTKNQVSLTCLVKGFYPSDIAVEWESNGQPENNYKT |
| | | TPPVLDSDGSFFLYSRLTVDKSRWQEGNVFSCSVMHEALHNHYTQKSL |
| | | SLSLGK |
| 83 | Ab3MC | EVQLQQSGPVLVKPGASVKMSCKASGYTFTDYYMNWVKQSHGKSLEWI |
| | | GVINPYNGGTSYNQKFKGKATLTVDKSSSTAYMELNSLTSEDSAVYYCA |
| | | RDLTGTGGAMDYWGQGTSVTVSSASTKGPSVFPLAPCSRSTSESTAAL |
| | | GCLVKDYFPEPVTVSWNSGALTSGVHTFPAVLQSSGLYSLSSVVTVPSS |
| | | SLGTKTYTCNVDHKPSNTKVDKRVESKYGPPCPPCPAPEFLGGPSVFLF |
| | | PPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAK |
| | | GQPREPQVYTLPPSQEEMTKNQVSLTCLVKGFYPSDIAVEWESNGQPE |
| | | NNYKTTPPVLDSDGSFFLYSRLTVDKSRWQEGNVFSCSVMHEALHNHY |
| | | TQKSLSLSLGK |
| 84 | Ab5MC | EVQLQQSGAELVKPGASVKLSCTASGFNIKDYYMHWVKQRTEQGLEWI |
| | | GRIDPEDGETKYAPKFQGKATITADTSSNTAYLQLSSLTSEDTAVYYCAL |
| | | LWLRRGRGYWGQGTTLTVSSASTKGPSVFPLAPCSRSTSESTAALGCL |
| | | VKDYFPEPVTVSWNSGALTSGVHTFPAVLQSSGLYSLSSVVTVPSSSLG |
| | | TKTYTCNVDHKPSNTKVDKRVESKYGPPCPPCPAPEFLGGPSVFLFPPK |
| | | PKDTLMISRTPEVTCVVVDVSQEDPEVQFNWYVDGVEVHNAKTKPREE |
| | | QFNSTYRVVSVLTVLHQDWLNGKEYKCKVSNKGLPSSIEKTISKAKGQP |
| | | REPQVYTLPPSQEEMTKNQVSLTCLVKGFYPSDIAVEWESNGQPENNY |
| | | KTTPPVLDSDGSFFLYSRLTVDKSRWQEGNVFSCSVMHEALHNHYTQK |
| | | SLSLSLGK |
| 85 | Ab6MC | QVQLQQSGAELMKPGASVKLSCKATGYTFTGYWIEWVKQRPGHGLEWI |
| | | GEILPGSGSTNYNEKFKGKATFTADTSSNTAYMQLSSLTTEDSAIYYCAR |
| | | LRGSSYPWYFDYWGQGTTLTVSSASTKGPSVFPLAPCSRSTSESTAAL |
| | | GCLVKDYFPEPVTVSWNSGALTSGVHTFPAVLQSSGLYSLSSVVTVPSS |
| | | SLGTKTYTCNVDHKPSNTKVDKRVESKYGPPCPPCPAPEFLGGPSVFLF |
| | | PPKPKDTLMISRTPEVTCVVVDVSQEDPEVQFNWYVDGVEVHNAKTKP |
| | | REEQFNSTYRVVSVLTVLHQDWLNGKEYKCKVSNKGLPSSIEKTISKAK |
| | | GQPREPQVYTLPPSQEEMTKNQVSLTCLVKGFYPSDIAVEWESNGQPE |
| | | NNYKTTPPVLDSDGSFFLYSRLTVDKSRWQEGNVFSCSVMHEALHNHY |
| | | TQKSLSLSLGK |
| 86 | Ab7MC | EVQLQQSGAELVKPGASVKLSCTASGFNIKDYYMHWVKQRTEQGLEWI |
| | | GRIDPEDGETKYAPKFQGKATITADTSSNTAYLQLSSLTSEDTAVYYCAR |
| | | GKTPFAYWGQGTLVTVSAASTKGPSVFPLAPCSRSTSESTAALGCLVKD |
| | | YFPEPVTVSWNSGALTSGVHTFPAVLQSSGLYSLSSVVTVPSSSLGTKT |
| | | YTCNVDHKPSNTKVDKRVESKYGPPCPPCPAPEFLGGPSVFLFPPKPKD |
| | | TLMISRTPEVTCVVVDVSQEDPEVQFNWYVDGVEVHNAKTKPREEQFN |
| | | STYRVVSVLTVLHQDWLNGKEYKCKVSNKGLPSSIEKTISKAKGQPREP |
| | | QVYTLPPSQEEMTKNQVSLTCLVKGFYPSDIAVEWESNGQPENNYKTTP |
| | | PVLDSDGSFFLYSRLTVDKSRWQEGNVFSCSVMHEALHNHYTQKSLSL |
| | | SLGK |
| 87 | Mouse Ab1 | QVQLQQPGAELVRPGSSVKLSCKASGYTFTSYWMHWVKQRPIQGLEWI |
| | HC | GNIDPSDSETHYNQKFKDKATLTVDKSSSTAYMQLSSLTSEDSAVYYCA |
| | | RGRNYWYFDVWGTGTTVTVSS |
| 88 | Humanized | QVQLVQSGAEVKKPGASVKVSCKASGYTFTSYWMHWVRQAPGQGLE |
| | Ab1 HC 1 | WMGNIDPSDSETHYAQKFQGRVTMTRDTSTSTVYMELSSLRSEDTAVY |
| | | YCARGRNYWYFDVWGTGTMVTVSS |
| 89 | Humanized | QVQLVQSGAEVKKPGASVKVSCKASGYTFTSYWMHWVRQAPGQGLE |
| | Ab1 HC 2 | WMGNIDPSDSETHYAQKFQGRVTMTVDTSTSTVYMELSSLRSEDTAVY |
| | | YCARGRNYWYFDVWGTGTMVTVSS |
| 90 | Humanized | QVQLVQSGAEVKKPGASVKVSCKASGYTFTSYWMHWVRQAPGQGLE |
| | Ab1 HC 3 | WMGNIDPSDSETHYAQKFQGRVTMTVDKSTSTVYMELSSLRSEDTAVY |
| | | YCARGRNYWYFDVWGTGTMVTVSS |
| 91 | Humanized | QVQLVQSGAEVKKPGASVKVSCKASGYTFTSYWMHWVRQAPGQGLE |
| | Ab1 HC 4 | WIGNIDPSDSETHYAQKFQGRATLTVDKSTSTVYMELSSLRSEDTAVYY |
| | | CARGRNYWYFDVWGTGTMVTVSS |
| 92 | Mouse Ab1 | DIVLTQSPASLAVSLGQRATISCRASESVDNYGISFMNWFQQKPGQPPK |
| | LC | LLIYAASNQGSGVPARFSGSGSGTDFSLNIHPMEEDDTAMYFCQQSKEV |
| | | PWTFGGGTKLEIK |
| 93 | Humanized | DIQMTQSPSSLSASVGDRVTITCRASESVDNYGISFMNWYQQKPGKAPK |
| | Ab1 LC 1 | LLIYAASNQGSGVPSRFSGSGSGTDFTLTISSLQPEDFATYYCQQSKEVP |
| | | WTFGGGTKLEIK |
| 94 | Humanized | DIQMTQSPSSLSASVGDRVTITCRASESVDNYGISFMNWFQQKPGKAPK |
| | Ab1 LC 2 | LLIYAASNQGSGVPSRFSGSGSGTDFTLTISSLQPEDFATYFCQQSKEVP |
| | | WTFGGGTKLEIK |
| 95 | Humanized | DIQMTQSPSSLSASVGDRVTITCRASESVDNYGISFMNWFQQKPGKPPK |
| | Ab1 LC 3 | LLIYAASNQGSGVPSRFSGSGSGTDFTLTISSLQPEDFATYYCQQSKEVP |
| | | WTFGGGTKLEIK |
| 96 | Mouse Ab2 | EVKLEQSGGGLVQPGGSMKLSCVASGFTFSNYWMNWVRQSPEKGLE |
| | HC | WVAQIRLKSDNYATHYAESVKGRFTISRDDSKSSVYLQMNNLRAEDTG |
| | | YYCTGVFTYWGQGTLVTVSA |
| 97 | Humanized | EVQLVESGGGLVKPGGSLRLSCAASGFTFSNYWMNWVRQAPGKGLEW |
| | Ab2 HC-1 | VGQIRLKSDNYATHYAAPVKGRFTISRDDSKNTLYLQMNSLKTEDTAVYY |
| | | CTTVFTYWGQGTLVTVSS |
| 98 | Humanized | EVQLVESGGGLVKPGGSLRLSCAASGFTFSNYWMNWVRQAPGKGLEW |
| | Ab2 HC-2 | VGQIRLKSDNYATHYAAPVKGRFTISRDDSKNTLYLQMNSLKTEDTAVYY |
| | | CTGVFTYWGQGTLVTVSS |
| 99 | Humanized | EVQLVESGGGLVKPGGSLRLSCAASGFTFSNYWMNWVRQAPGKGLEW |
| | Ab2 HC-3 | VAQIRLKSDNYATHYAAPVKGRFTISRDDSKNTVYLQMNSLKTEDTAVYY |
| | | CTGVFTYWGQGTLVTVSS |
| 100 | Humanized | EVQLLESGGGLVQPGGSLRLSCAASGFTFSNYWMNWVRQAPGKGLEW |
| | Ab2 HC-4 | VSQIRLKSDNYATHYADSVKGRFTISRDNSKNTLYLQMNSLRAEDTAVYY |
| | | CAKVFTYWGQGTLVTVSS |
| 101 | Humanized | EVQLLESGGGLVQPGGSLRLSCAASGFTFSNYWMNWVRQAPGKGLEW |
| | Ab2 HC-5 | VSQIRLKSDNYATHYADSVKGRFTISRDNSKNTLYLQMNSLRAEDTAVYY |
| | | CTGVFTYWGQGTLVTVSS |
| 102 | Humanized | EVQLLESGGGLVQPGGSLRLSCAASGFTFSNYWMNWVRQAPGKGLEW |
| | Ab2 HC-6 | VAQIRLKSDNYATHYADSVKGRFTISRDDSKNTVYLQMNSLRAEDTAVY |
| | | YCTGVFTYWGQGTLVTVSS |
| 103 | Mouse Ab2 | NIVLTQSPASLAVSLGQRATISCRTSESVDSYGNSFMHWYQQKPGQPPK |
| | LC | LLIYLASNLESGVPARFSGSGSRTDFTLTIDPVEADDAATYYCQQNNEDP |
| | | MYTFGGGTKLEIK |
| 104 | Humanized | DIVMTQSPDSLAVSLGERATINCRTSESVDSYGNSFMHWYQQKPGQPP |
| | Ab2 LC-1 | KLLIYLASNLESGVPDRFSGSGSGTDFTLTISSLQAEDVAVYYCQQNNED |
| | | PMYTFGGGTKVEIK |
| 105 | Humanized | DIVMTQSPDSLAVSLGERATINCRTSESVDSYGNSFMHWYQQKPGQPP |
| | Ab2 LC-2 | KLLIYLASNLESGVPDRFSGSGSRTDFTLTISSLQAEDVAVYYCQQNNED |
| | | PMYTFGGGTKVEIK |
| 106 | Humanized | DIVLTQSPDSLAVSLGERATINCRTSESVDSYGNSFMHWYQQKPGQPPK |
| | Ab2 LC-3 | LLIYLASNLESGVPDRFSGSGSRTDFTLTISSLQAEDVAVYYCQQNNEDP |
| | | MYTFGGGTKVEIK |
| 107 | Mouse Ab3 | EVQLQQSGPVLVKPGASVKMSCKASGYTFTDYYMNWVKQSHGKSLEWI |
| | HC | GVINPYNGGTSYNQKFKGKATLTVDKSSSTAYMELNSLTSEDSAVYYCA |
| | | RDLTGTGGAMDYWGQGTSVTVSS |
| 108 | Humanized | QVQLVQSGAEVKKPGASVKVSCKASGYTFTDYYMNWVRQAPGQGLEW |
| | Ab3 HC-1 | MGVINPYNGGTSYAQKFQGRVTMTRDTSTSTVYMELSSLRSEDTAVYY |
| | | CARDLTGTGGAMDYWGQGTLVTVSS |
| 109 | Humanized | QVQLVQSGAEVKKPGASVKVSCKASGYTFTDYYMHWVRQAPGQGLEW |
| | Ab3 HC-2 | MGVINPYNGGTSYAQKFQGRVTMTVDKSTSTVYMELSSLRSEDTAVYY |
| | | CARDLTGTGGAMDYWGQGTLVTVSS |
| 110 | Humanized | QVQLVQSGAEVKKPGASVKVSCKASGYTFTDYYMHWVRQAPGQGLEW |
| | Ab3 HC-3 | IGVINPYNGGTSYAQKFQGRATLTVDKSTSTAYMELSSLRSEDTAVYYCA |
| | | RDLTGTGGAMDYWGQGTLVTVSS |
| 111 | Humanized | QVQLVQSGAEVKKPGASVKVSCKASGYTFTDYYMHWVRQAPGQSLEW |
| | Ab3 HC-4 | IGVINPYNGGTSYAQKFQGRATLTVDKSTSTAYMELSSLRSEDTAVYYCA |
| | | RDLTGTGGAMDYWGQGTLVTVSS |
| 112 | Mouse Ab3 | QIVLTQSPAILSASPGEKVTMTCRASSSVSYMHWYQQKPGSSPKPWIYA |
| | LC | TSNLASGVPARFSGSGSGTSYSLTISRVEAEDAATYYCQQWSSNPLTFG |
| | | AGTKLELK |
| 113 | Humanized | EIVLTQSPGTLSLSPGERATLSCRASSSVSYMHWYQQKPGQAPRLLIYA |
| | Ab3 LC-1 | TSNLASGIPDRFSGSGSGTDFTLTISSLEPEDFAVYYCQQWSSNPLTFG |
| | | GGTKVEIK |
| 114 | Humanized | EIVLTQSPGTLSLSPGERATLSCRASSSVSYMHWYQQKPGQAPRPWIYA |
| | Ab3 LC-2 | TSNLASGIPDRFSGSGSGTDFTLTISSLEPEDFAVYYCQQWSSNPLTFG |
| | | GGTKVEIK |
| 115 | Humanized | EIVLTQSPGTLSLSPGERATLSCRASSSVSYMHWYQQKPGQAPRPWIYA |
| | Ab3 LC-3 | TSNLASGIPDRFSGSGSGTDYTLTISSLEPEDFAVYYCQQWSSNPLTFG |
| | | GGTKVEIK |
| 116 | Humanized | EIVLTQSPGTLSLSPGERATLSCRASSSVSYMHWYQQKPGQSPRPWIYA |
| | Ab3 LC-4 | TSNLASGIPDRFSGSGSGTDYTLTISSLEPEDFAVYYCQQWSSNPLTFG |
| | | GGTKVEIK |
| 117 | Mouse Ab5 | EVQLQQSGAELVKPGASVKLSCTASGFNIKDYYMHWVKQRTEQGLEWI |
| | HC | GRIDPEDGETKYAPKFQGKATITADTSSNTAYLQLSSLTSEDTAVYYCAL |
| | | LWLRRGRGYWGQGTTLTVSS |
| 118 | Humanized | QVQLVQSGAEVKKPGSSVKVSCKASGFNIKDYYMHWVRQAPGQGLEW |
| | Ab5 HC-1 | MGRIDPEDGETKYAQKFQGRVTITADESTSTAYMELSSLRSEDTAVYYC |
| | | ARLWLRRGRGYWGQGTLVTVSS |
| 119 | Humanized | QVQLVQSGAEVKKPGSSVKVSCKASGFNIKDYYMHWVRQAPGQGLEW |
| | Ab5 HC-2 | MGRIDPEDGETKYAQKFQGRVTITADTSTSTAYMELSSLRSEDTAVYYC |
| | | ALLWLRRGRGYWGQGTLVTVSS |
| 120 | Humanized | QVQLVQSGAEVKKPGSSVKVSCKASGFNIKDYYMHWVRQAPGQGLEWI |
| | Ab5 HC-3 | GRIDPEDGETKYAQKFQGRATITADTSTSTAYMELSSLRSEDTAVYYCAL |
| | | LWLRRGRGYWGQGTLVTVSS |
| 121 | Mouse Ab5 | DIQMTQSPSSLSASLGERVSLTCRASQEISGYLSWLQQKPDGTIKRLIYA |
| | LC | ASTLDSGVPKRFSGSRSGSDYSLTISSLESEDFADYYCLQYASYPPTFG |
| | | AGTKLELK |
| 122 | Humanized | DIQMTQSPSSLSASVGDRVTITCRASQEISGYLSWYQQKPGKAPKLLIYA |
| | Ab5 LC-1 | ASTLDSGVPSRFSGSGSGTDFTLTISSLQPEDFATYYCLQYASYPPTFGG |
| | | GTKVEIK |
| 123 | Humanized | DIQMTQSPSSLSASVGDRVTITCRASQEISGYLSWLQQKPGKAPKRLIYA |
| | Ab5 LC-2 | ASTLDSGVPSRFSGSRSGTDFTLTISSLQPEDFATYYCLQYASYPPTFGG |
| | | GTKVEIK |
| 124 | Humanized | DIQMTQSPSSLSASVGDRVTITCRASQEISGYLSWLQQKPGKTIKRLIYAA |
| | Ab5 LC-3 | STLDSGVPSRFSGSRSGTDFTLTISSLQPEDFATYYCLQYASYPPTFGG |
| | | GTKVEIK |
| 125 | Humanized | DIQMTQSPSSLSASVGDRVTITCRASQEISGYLSWLQQKPGKTIKRLIYAA |
| | Ab5 LC-4 | STLDSGVPSRFSGSRSGTDYTLTISSLQPEDFATYYCLQYASYPPTFGG |
| | | GTKVEIK |
| 126 | Mouse Ab6 | QVQLQQSGAELMKPGASVKLSCKATGYTFTGYWIEWVKQRPGHGLEWI |
| | HC | GEILPGSGSTNYNEKFKGKATFTADTSSNTAYMQLSSLTTEDSAIYYCAR |
| | | LRGSSYPWYFDYWGQGTTLTVSS |
| 127 | Humanized | QVQLVQSGAEVKKPGSSVKVSCKASGYTFTGYWIEWVRQAPGQGLEW |
| | Ab6 HC-1 | MGEILPGSGSTNYAQKFQGRVTITADESTSTAYMELSSLRSEDTAVYYC |
| | | ARLRGSSYPWYFDYWGQGTLVTVSS |
| 128 | Humanized | QVQLVQSGAEVKKPGSSVKVSCKASGYTFTGYWIEWVRQAPGQGLEW |
| | Ab6 HC-2 | MGEILPGSGSTNYAQKFQGRVTFTADTSTSTAYMELSSLRSEDTAVYYC |
| | | ARLRGSSYPWYFDYWGQGTLVTVSS |
| 129 | Humanized | QVQLVQSGAEVKKPGSSVKVSCKASGYTFTGYWIEWVRQAPGQGLEWI |
| | Ab6 HC-3 | GEILPGSGSTNYAQKFQGRATFTADTSTSTAYMELSSLRSEDTAVYYCA |
| | | RLRGSSYPWYFDYWGQGTLVTVSS |
| 130 | Mouse Ab6 | DIQMTQSPASLSVSVGETVTITCRASENIYSNLAWYQQKQGKSPQLLVYA |
| | LC | ATNLADGVPSRFSGSGSGTQYSLKINSLQSEDFGSYYCQHFWGTPYTF |
| | | GGGTKLEIK |
| 131 | Humanized | DIQMTQSPSSLSASVGDRVTITCRASENIYSNLAWYQQKPGKAPKLLIYA |
| | Ab6 LC-1 | ATNLADGVPSRFSGSGSGTDFTLTISSLQPEDFATYYCQHFWGTPYTFG |
| | | GGTKVEIK |
| 132 | Humanized | DIQMTQSPSSLSASVGDRVTITCRASENIYSNLAWYQQKPGKAPKLLVYA |
| | Ab6 LC-2 | ATNLADGVPSRFSGSGSGTDYTLTISSLQPEDFATYYCQHFWGTPYTFG |
| | | GGTKVEIK |
| 133 | Humanized | DIQMTQSPSSLSASVGDRVTITCRASENIYSNLAWYQQKPGKSPKLLVYA |
| | Ab6 LC-3 | ATNLADGVPSRFSGSGSGTDYTLTISSLQPEDFATYYCQHFWGTPYTFG |
| | | GGTKVEIK |
| 134 | Mouse Ab7 | EVQLQQSGAELVKPGASVKLSCTASGFNIKDYYMHWVKQRTEQGLEWI |
| | HC | GRIDPEDGETKYAPKFQGKATITADTSSNTAYLQLSSLTSEDTAVYYCAR |
| | | GKTPFAYWGQGTLVTVSÅ |
| 135 | Humanized | QVQLVQSGAEVKKPGSSVKVSCKASGFNIKDYYMHWVRQAPGQGLEW |
| | Ab7 HC-1 | MGRIDPEDGETKYAQKFQGRVTITADESTSTAYMELSSLRSEDTAVYYC |
| | | ARGKTPFAYWGQGTLVTVSS |
| 136 | Humanized | QVQLVQSGAEVKKPGSSVKVSCKASGFNIKDYYMHWVRQAPGQGLEW |
| | Ab7 HC-2 | MGRIDPEDGETKYAQKFQGRVTITADTSTNTAYMELSSLRSEDTAVYYC |
| | | ARGKTPFAYWGQGTLVTVSS |
| 137 | Humanized | QVQLVQSGAEVKKPGSSVKVSCKASGFNIKDYYMHWVRQAPGQGLEWI |
| | Ab7 HC-3 | GRIDPEDGETKYAQKFQGKATITADTSSNTAYMELSSLRSEDTAVYYCA |
| | | RGKTPFAYWGQGTLVTVSS |
| 138 | Mouse Ab7 | DIQMTQSPSSLSASLGERVSLTCRASQEISGYLSWLQQKPDGTIKRLIYA |
| | LC | ASTLDFGVPKRFSGSRFGSDYSLTISSLESEDFADYYCLQYDSYPFTFGS |
| | | GTKLEIK |
| 139 | Humanized | DIQMTQSPSSLSASVGDRVTITCRASQEISGYLSWYQQKPGKAPKLLIYA |
| | Ab7 LC-1 | ASTLDFGVPSRFSGSGSGTDFTLTISSLQPEDFATYYCLQYDSYPFTFGG |
| | | GTKVEIK |
| 140 | Humanized | DIQMTQSPSSLSASVGDRVTITCRASQEISGYLSWLQQKPGKAPKRLIYA |
| | Ab7 LC-2 | ASTLDFGVPSRFSGSRSGTDFTLTISSLQPEDFATYYCLQYDSYPFTFGG |
| | | GTKVEIK |
| 141 | Humanized | DIQMTQSPSSLSASVGDRVTITCRASQEISGYLSWLQQKPGKAPKRLIYA |
| | Ab7 LC-3 | ASTLDFGVPSRFSGSRSGSDFTLTISSLQPEDFATYYCLQYDSYPFTFGG |
| | | GTKVEIK |
| 142 | Humanized | DIQMTQSPSSLSASVGDRVTITCRASQEISGYLSWLQQKPGKTIKRLIYAA |
| | Ab7 LC-4 | STLDFGVPSRFSGSRSGSDFTLTISSLQPEDFATYYCLQYDSYPFTFGG |
| | | GTKVEIK |
| 143 | F87M TTR | GPTGTGESKCPLMVKVLDAVRGSPAINVAVHVFRKAADDTWEPFASGK |
| | | TSESGELHGLTTEEEFVEGIYKVEIDTKSYWKALGISPMHEHAEVVFTAN |
| | | DSGPRRYTIAALLSPYSYSTTAVVTNPKE |
| 144 | L110M | GPTGTGESKCPLMVKVLDAVRGSPAINVAVHVFRKAADDTWEPFASGK |
| | TTR | TSESGELHGLTTEEEFVEGIYKVEIDTKSYWKALGISPFHEHAEVVFTAND |
| | | SGPRRYTIAAMLSPYSYSTTAVVTNPKE |
| 145 | V30M TTR | GPTGTGESKCPLMVKVLDAVRGSPAINVAMHVFRKAADDTWEPFASGK |
| | | TSESGELHGLTTEEEFVEGIYKVEIDTKSYWKALGISPFHEHAEVVFTAND |
| | | SGPRRYTIAALLSPYSYSTTAVVTNPKE |
| 146 | D38A TTR | GPTGTGESKCPLMVKVLDAVRGSPAINVAVHVFRKAAADTWEPFASGK |
| | | TSESGELHGLTTEEEFVEGIYKVEIDTKSYWKALGISPFHEHAEVVFTAND |
| | | SGPRRYTIAALLSPYSYSTTAVVTNPKE |
| 147 | V122I TTR | GPTGTGESKCPLMVKVLDAVRGSPAINVAVHVFRKAADDTWEPFASGK |
| | | TSESGELHGLTTEEEFVEGIYKVEIDTKSYWKALGISPFHEHAEVVFTAND |
| | | SGPRRYTIAALLSPYSYSTTAVITNPKE |
| 148 | S112I TTR | GPTGTGESKCPLMVKVLDAVRGSPAINVAVHVFRKAADDTWEPFASGK |
| | | TSESGELHGLTTEEEFVEGIYKVEIDTKSYWKALGISPFHEHAEVVFTAND |
| | | SGPRRYTIAALLIPYSYSTTAVVTNPKE |
| 149 | V30M | GPTGTGESKCPLMVKVLDAVRGSPAINVAMHVFRKAADDTWEPFASGK |
| | F87M | TSESGELHGLTTEEEFVEGIYKVEIDTKSYWKALGISPMHEHAEVVFTAN |
| | | DSGPRRYTIAAMLSPYSYSTTAVVTNPKE |

What is claimed is:

1. An isolated antibody that binds to human transthyretin (TTR) wherein the antibody:
(a) binds to human TTR with an EC₅₀ of less than about 200 nM as determined in a sandwich ELISA; and/or
(b) binds to human TTR with a K_{D} of less than about 20 nM as determined in a biolayer interferometry assay.

2. The antibody of claim 1 wherein the human TTR is in a monomeric form.

3. The antibody of claim 2 wherein the antibody binds monomeric TTR with an EC₅₀ of less than about 20 nM as determined in an indirect ELISA.

4. The antibody of any one of claims 1-3 wherein the antibody prevents aggregation of TTR.

5. The antibody of any one of claims 1-4 wherein the antibody binds a folding intermediate of TTR.

6. The antibody of claim 1 comprising:
a heavy chain variable domain (VH) comprising
(a) a CDR-H1 comprising the amino acid sequence of any one of SEQ ID NOs: 2-8;
(b) a CDR-H2 comprising the amino acid sequence of any one of SEQ ID NOs: 9-15; and
(c) a CDR-H3 comprising the amino acid sequence of any one of SEQ ID NOs: 16-22; and
a light chain variable domain (VL) comprising
(d) a CDR-L1 comprising the amino acid sequence of any one of SEQ ID NOs: 23-29;
(e) a CDR-L2 comprising the amino acid sequence of any one of SEQ ID NOs: 30-36; and
(f) a CDR-L3 comprising the amino acid sequence of any one of SEQ ID NOs: 37-43.

7. An antibody that binds to human transthyretin (TTR), the antibody comprising
a heavy chain variable domain (VH) comprising
(a) a CDR-H1 comprising the amino acid sequence of any one of SEQ ID NOs: 2-8;
(b) a CDR-H2 comprising the amino acid sequence of any one of SEQ ID NOs: 9-15; and
(c) a CDR-H3 comprising the amino acid sequence of any one of SEQ ID NOs: 16-22; and
a light chain variable domain (VL) comprising
(d) a CDR-L1 comprising the amino acid sequence of any one of SEQ ID NOs: 23-29;
(e) a CDR-L2 comprising the amino acid sequence of any one of SEQ ID NOs: 30-36; and
(f) a CDR-L3 comprising the amino acid sequence of any one of SEQ ID NOs: 37-43.

8. The antibody of any one of claims 1-4 comprising:
(a) a CDR-H1 comprising the amino acid sequence of SEQ ID NO: 2;
(b) a CDR-H2 comprising the amino acid sequence of SEQ ID NO: 9; and
(c) a CDR-H3 comprising the amino acid sequence of SEQ ID NO: 16; and
a light chain variable domain (VL) comprising
(d) a CDR-L2 comprising the amino acid sequence of SEQ ID NO: 23;
(e) a CDR-L2 comprising the amino acid sequence of SEQ ID NO: 30; and
(f) a CDR-L3 comprising the amino acid sequence of SEQ ID NO: 37.

9. The antibody of any one of claims 1-4 comprising:
a heavy chain variable domain (VH) comprising
(a) a CDR-H1 comprising the amino acid sequence of SEQ ID NO: 3;
(b) a CDR-H2 comprising the amino acid sequence of SEQ ID NO: 10; and
(c) a CDR-H3 comprising the amino acid sequence of SEQ ID NO: 17; and
a light chain variable domain (VL) comprising
(d) a CDR-L2 comprising the amino acid sequence of SEQ ID NO: 24;
(e) a CDR-L2 comprising the amino acid sequence of SEQ ID NO: 31; and
(f) a CDR-L3 comprising the amino acid sequence of SEQ ID NO: 38.

10. The antibody of any one of claims 1-4 comprising:
a heavy chain variable domain (VH) comprising
(a) a CDR-H1 comprising the amino acid sequence of SEQ ID NO: 4;
(b) a CDR-H2 comprising the amino acid sequence of SEQ ID NO: 11; and
(c) a CDR-H3 comprising the amino acid sequence of SEQ ID NO: 18; and
a light chain variable domain (VL) comprising
(d) a CDR-L2 comprising the amino acid sequence of SEQ ID NO: 25;
(e) a CDR-L2 comprising the amino acid sequence of SEQ ID NO: 32; and
(f) a CDR-L3 comprising the amino acid sequence of SEQ ID NO: 39.

11. The antibody of any one of claims 1-4 comprising:
a heavy chain variable domain (VH) comprising
(a) a CDR-H1 comprising the amino acid sequence of SEQ ID NO: 5;
(b) a CDR-H2 comprising the amino acid sequence of SEQ ID NO: 12; and
(c) a CDR-H3 comprising the amino acid sequence of SEQ ID NO: 19; and
a light chain variable domain (VL) comprising
(d) a CDR-L2 comprising the amino acid sequence of SEQ ID NO: 26;
(e) a CDR-L2 comprising the amino acid sequence of SEQ ID NO: 33; and
(f) a CDR-L3 comprising the amino acid sequence of SEQ ID NO: 40.

12. The antibody of any one of claims 1-4 comprising:
a heavy chain variable domain (VH) comprising
(a) a CDR-H1 comprising the amino acid sequence of SEQ ID NO: 6;
(b) a CDR-H2 comprising the amino acid sequence of SEQ ID NO: 13; and
(c) a CDR-H3 comprising the amino acid sequence of SEQ ID NO: 20; and
a light chain variable domain (VL) comprising
(d) a CDR-L2 comprising the amino acid sequence of SEQ ID NO: 27;
(e) a CDR-L2 comprising the amino acid sequence of SEQ ID NO: 34; and
(f) a CDR-L3 comprising the amino acid sequence of SEQ ID NO: 41.

13. The antibody of any one of claims 1-4 comprising:
a heavy chain variable domain (VH) comprising
(a) a CDR-H1 comprising the amino acid sequence of SEQ ID NO: 7;
(b) a CDR-H2 comprising the amino acid sequence of SEQ ID NO: 14; and
(c) a CDR-H3 comprising the amino acid sequence of SEQ ID NO: 21; and
a light chain variable domain (VL) comprising
(d) a CDR-L2 comprising the amino acid sequence of SEQ ID NO: 28;
(e) a CDR-L2 comprising the amino acid sequence of SEQ ID NO: 35; and
(f) a CDR-L3 comprising the amino acid sequence of SEQ ID NO: 42.

14. The antibody of any one of claims 1-4 comprising:
a heavy chain variable domain (VH) comprising
(a) a CDR-H1 comprising the amino acid sequence of SEQ ID NO: 8;
(b) a CDR-H2 comprising the amino acid sequence of SEQ ID NO: 15;
(c) a CDR-H3 comprising the amino acid sequence of SEQ ID NO: 22; and
a light chain variable domain (VL) comprising
(d) a CDR-L2 comprising the amino acid sequence of SEQ ID NO: 29;
(e) a CDR-L2 comprising the amino acid sequence of SEQ ID NO: 36; and
(f) a CDR-L3 comprising the amino acid sequence of SEQ ID NO: 43.

15. The antibody of any one of claims 1-14 which is a monoclonal antibody.

16. The antibody of any one of claims 1-15 which is a humanized or chimeric antibody.

17. The antibody of claim 16 wherein the VH domain comprises a sequence comprising at least 85% sequence identity to the sequence of any one of SEQ ID NOs: 46-49.

18. The antibody of claim 16 wherein the VH domain comprises the sequence of any one of SEQ ID NOs: 46-49.

19. The antibody of claim 16 wherein the VH domain comprises a sequence comprising at least 85% sequence identity to the sequence of any one of SEQ ID NOs: 55-60.

20. The antibody of claim 16 wherein the VH domain comprises the sequence of any one of SEQ ID NOs: 55-60.

21. The antibody of claim 16 wherein the VL domain comprises at least 85% sequence identity to the sequence of any one of SEQ ID NOs: 51-53.

22. The antibody of claim 16 wherein the VL domain comprises the sequence of any one of SEQ ID NOs: 51-53.

23. The antibody of claim 16 wherein the VL domain comprises a sequence comprising at least 85% sequence identity to the sequence of any one of SEQ ID NOs: 62-64.

24. The antibody of claim 16 wherein the VL domain comprises the sequence of any one of SEQ ID NOs: 62-64.

25. The antibody of any one of claims 1-24 which is an antibody fragment that binds to a monomeric form or a folding intermediate form of human TTR.

26. The antibody of any one of claims 1-25 that does not bind to a peptide having an amino acid sequence selected from any one of GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69), LSPYSY (SEQ ID NO: 70), SYSTTAVV (SEQ ID NO: 71), YTIAALLSPYSYSTTAVV (SEQ ID NO: 72), LGISPMHEHAE (SEQ ID NO: 73) and RRYTIAAMLSPYS (SEQ ID NO: 74).

27. The antibody of claim 26 that binds to a conformational epitope.

28. The antibody of any of claims 1-27 which is an IgG4 antibody.

29. The antibody of any one of claims 1-28 wherein the antibody binds human TTR with an EC₅₀ of less than about 150 nM, or less than about 100 nM, or less than about 50 nM, or less than about 20 nM, as determined in a sandwich ELISA.

30. The antibody of any one of claims 1-29, wherein the antibody binds human TTR with an EC₅₀ of less than about 20 nM, or less than about 5 nM, or less than about 2 nM, or less than about 1 nM, as determined using an Indirect ELISA.

31. An antibody that specifically binds human TTR and that competes for binding to human TTR with the antibody of any one of claims 1-30.

32. An isolated nucleic acid comprising a nucleotide sequence encoding the antibody of any of claims 1-31.

33. An isolated host cell comprising the nucleic acid of claim 32.

34. An isolated host cell that express the antibody of any one of claims 1-31.

35. A method of producing an antibody that binds to human TTR comprising culturing the host cell of claim 33 or claim 34 under conditions suitable for the expression of the antibody.

36. The method of claim 35, further comprising recovering the antibody from the host cell or host cell culture.

37. An antibody produced by the method of claim 35 or 36.

38. A composition comprising the antibody of any of claims 1-31 and 37.

39. The antibody of any one of claims 1-31 and 37, or the composition of claim 38 for use in treating, preventing, or diagnosing a disease associated with associated with TTR aggregation.

40. The antibody of any one of claims 1-31 and 37, or the composition of claim 38 for use in treating TTR amyloidosis.

41. The antibody of claim 40 wherein the TTR amyloidosis is transthyretin amyloidosis (ATTR) cardiomyopathy (ATTR-CM) or transthyretin amyloidosis (ATTR) polyneuropathy (ATTR-PN).

42. The antibody of any one of claims 1-31 and 37, or the composition of claim 38 for use in treating peripheral TTR amyloidosis, ocular amyloid angiopathy or cerebral amyloid angiopathy.

43. The antibody of any one of claims 1-31 and 37, or the composition of claim 38 for use in treating a disease selected from Familial Amyloid Polyneuropathy (AP), Familial Amyloid Cardiomyopathy (FAC), ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia.

44. A method of treating a subject having a disease associated with TTR amyloidosis, the method comprising administering to the subject an effective amount of the antibody of any one of claims 1-31 and 37, or the composition of claim 38.

45. The method of claim 44 wherein the disease is TTR amyloidosis.

46. A method of treating a subject having a disease selected from Familial Amyloid Polyneuropathy (AP), Familial Amyloid Cardiomyopathy (FAC), ATTR-PN and ATTR-CM, hATTR or wild-type ATTR (wtATTR), Senile Systemic Amyloidosis (SSA), systemic familial amyloidosis, leptomeningeal/Central Nervous System (CNS) amyloidosis including Alzheimer disease, TTR-related ocular amyloidosis, TTR-related renal amyloidosis, TTR-related hyperthyroxinemia, TTR-related ligament amyloidosis including carpal tunnel syndrome, rotator cuff tears and lumbar spinal stenosis, and preeclampsia, the method comprising administering to the subject an effective amount of the antibody of any one of claims 1-31 and 37, or the composition of claim 38.

47. The antibody of any one of claims 1-31 and 37 further comprising a detectable label.

48. A method of measuring monomeric transthyretin (TTR) in a sample, the method comprising contacting the sample with the antibody of any one of claims 1-31, 37, and 47, and measuring the amount of antibody bound to TTR in the sample.

49. The method according to claim 48, wherein the measuring comprises contacting the sample with a secondary antibody and measuring a complex formed thereby.

50. The method according to claim 49 that comprises using TTR monomer antibodies Ab3 and Ab5, with one of said antibodies used as a capture antibody and one of said antibodies used as a detection antibody.

51. A method of diagnosing a disease associated with transthyretin (TTR) amyloidosis, or monitoring the treatment of the disease with an anti-TTR therapy comprising assaying the level of monomeric TTR in a sample of a body fluid from a subject, wherein the presence or an elevated level of monomeric TTR in the sample of the subject compared to a control indicates the presence of a disease associated with TTR amyloidosis.

52. The method of claim 51 wherein the monomeric TTR binds to the antibody of any one of claims 1-31 and 37.

53. A method comprising:
contacting a sample from a subject with a sample of a bodily fluid from a subject having or suspected of having transthyretin (TTR) amyloidosis; and
detecting or measuring monomeric TTR in the sample.

54. The method according to claim 53, wherein the contacting step uses a contacting antibody selected from TTR monomer antibodies Ab3 and Ab5.

55. The method according to claim 54, wherein the detecting or measuring step comprises contacting a complex formed by the contacting antibody and the TTR with a detecting antibody selected from TTR monomer antibodies Ab3 and Ab5, wherein the contacting antibody is different from the detecting antibody.

56. The method according to any one of claims 53-55, further comprising initiating anti-TTR therapy based on detecting or measuring the monomeric TTR in the sample.

57. The method according to any one of claims 53-55, further comprising adjusting the dose of an anti-TTR therapy based on the measurement of the monomeric TTR in the sample.

58. The method according to claim 56 or 57, wherein the anti-TTR therapy comprises administering to the subject an antibody according to any one of claims 1-31 and 37 or a composition according to claim 38.

59. A composition comprising the antibody of any one of claims 1-31 and 37 wherein the composition (i) is a pharmaceutical composition and further comprises a pharmaceutically acceptable carrier, or (ii) a diagnostic composition.

60. The composition of claim 59 wherein the diagnostic composition further comprises reagents conventionally used in immuno-based or antibody-based diagnostic methods.

61. The composition of claim 59 wherein the diagnostic method measures a TTR monomeric form.

62. The composition of claim 59 wherein the diagnostic method uses the Quanterix platform.

63. The composition of claim 59 wherein the diagnostic method is a Western gel.

64. An isolated antibody which recognizes a conformational epitope on monomeric TTR prepared from F87M L110M TTR that is not present on tetrameric TTR and physiological, cellular full-length TTR.

65. An isolated antibody which recognizes a TTR mutation selected from any of V30M, D38A and V1221, wherein, the antibody specifically binds TTR comprising the amino acid sequence set forth in any of SEQ ID NOs: 145-147.

66. The antibody of claim 65 wherein the TTR mutation is D38A and/or V1221, wherein the antibody specifically binds TTR comprising the amino acid sequence set forth in SEQ ID NO: 146 and/or 147.

67. The antibody of claim 16 wherein the VH domain comprises at least 85% sequence identity with any of the sequences set forth in FIGS. 6A-6B, 7, 14, and 18-21 (SEQ ID NOS. 81-142).

68. The antibody of claim 16 wherein the VL domain comprises at least 85% sequence identity with any of the sequences set forth in FIGS. 6A-6B, 7, 14, and 18-21 (SEQ ID NOs. 81-142).

69. The antibody of any one of claims 1-31 and 37, wherein the antibody specifically binds to TTR comprising the amino acid sequence set forth in any one of SEQ ID NOs. 80 and 143-147 and 149-152.

70. The antibody of claim 69, wherein the antibody specifically binds to TTR comprising the amino acid sequence set forth in SEQ ID NO: 80.

71. A method of producing a cell that produces an antibody that specifically binds to a monomeric form of human transthyretin (TTR), the method comprising:
(a) immunizing a non-human animal with a monomeric form of TTR;
(b) screening blood or tissue from the animal for an antibody which specifically binds to the monomeric form of human TTR but does not bind to wild-type tetrameric TTR or to a dimeric or tetrameric form of TTR; and
(c) identifying and isolating a cell from the animal that produces the antibody that specifically binds to the monomeric form of human TTR but does not bind to wild-type tetrameric TTR or to a dimeric or tetrameric form of TTR.

72. The method of claim 71, wherein the antibody-producing cell is recovered by removal of spleen tissue, lymph nodes or bone marrow of the animal.

73. The method of any one of claims 71-72, wherein the antibody-producing cell is a B cell, a T cell, or a stem cell.

74. The method of any one of claims 71-73, wherein the animal is a mouse, a rat, a guinea pig, or a rabbit.

75. The method of any one of claims 71-74, further comprising immortalizing the isolated cell.

76. The method of claim 75, comprising producing a hybridoma cell by somatic fusion of the cell to a myeloma cell.

77. The method of any one of claims 71-76, further comprising culturing the antibody-producing cell.

78. The method of any one of claims 71-77, wherein the antibody specifically binds to TTR comprising the amino acid sequence set forth in any one of SEQ ID NOs: 80, 143, and 144.

79. The method of any one of claims 71-78, wherein the antibody does not bind to a peptide having an amino acid sequence consisting of a sequence selected from any one of GPTGTGESKCPL (SEQ ID NO: 65), MVKVLDAVRGSPA (SEQ ID NO: 66), PFHEHAEVVFTA (SEQ ID NO: 67), EHAEVVFTA (SEQ ID NO: 68), YTIAALLS (SEQ ID NO: 69), LSPYSY (SEQ ID NO: 70), SYSTTAVV (SEQ ID NO: 71), YTIAALLSPYSYSTTAVV (SEQ ID NO: 72), LGISPMHEHAE (SEQ ID NO: 73) and RRYTIAAMLSPYS (SEQ ID NO: 74).

80. The method of any one of claims 71-79, wherein the monomeric form of human TTR used to immunize the animal is any one of
TTR F87M L110M comprising the amino acid sequence set forth in SEQ ID NO: 80;
TTR F87M comprising the amino acid sequence set forth in SEQ ID NO: 143; and
TTR L110M comprising the amino acid sequence set forth in SEQ ID NO: 144.

81. An antibody produced by the method of any one of claims 71-80.

82. A method for producing a monoclonal antibody that specifically binds to a monomeric form of human transthyretin (TTR), said method comprising:
(a) introducing into a non-human animal at least one of any one of
TTR F87M L110M comprising the amino acid sequence set forth in SEQ ID NO: 80;
TTR F87M comprising the amino acid sequence set forth in SEQ ID NO: 143; and
TTR L110M comprising the amino acid sequence set forth in SEQ ID NO: 144;
(b) recovering antibody-producing cells from the animal and rendering these cells into a single cell suspension;
(c) generating an immortalized cell line from the single cell suspension;
(d) screening the supernatant of the immortalized cell line for the presence of an antibody having binding specifically for a monomeric form of human TTR; and
(e) selecting, as the monoclonal antibody, an antibody that specifically binds to the monomeric form of human TTR.

83. The method of claim 82, wherein said animal is a mouse, a rat, a guinea pig or a rabbit.

84. The method of claim 82 or 83, wherein the antibody-producing cells are B cells, T cells or stem cells.

85. The method of any one of claims 82-84, wherein the antibody-producing cells are recovered by removal of spleen tissue, lymph nodes or bone marrow of the animal.

86. The method of any one of claims 82-85, wherein the immortalized cell line is a hybridoma cell line produced by somatic fusion of the cells in the single cell suspension to myeloma cells.

87. An antibody produced by the method of any one of claims 82-86.

88. A method comprising:
(a) assaying a library of molecules for a molecule peptide or polypeptide that:
(i) binds one or more monomeric TTR peptides having amino acid an amino acid sequence selected from SEQ ID NOs: 80 and 143-147 and 149-152; and
(ii) exhibits substantially no binding affinity for a tetrameric human TTR protein having the amino acid sequence of SEQ ID NO: 153.
(b) identifying a molecule that exhibits the binding properties of (i) and (ii); and
(c) isolating the molecule identified in (b), or isolating a cell that expresses said molecules.

89. The method according to claim 88, further comprising assaying to identify a molecule that exhibits the binding properties of (i) and (ii) and that (iii) fails to bind to a peptide having an amino acid sequence consisting of SEQ ID NO: 65, 66, 67, 68, 69, 70, 71, 72, 73, or 74.

90. The method according to claim 88 or 89, wherein molecules comprise antibodies or antigen-binding fragments of antibodies.

91. The method according to claim 90, wherein the isolating step comprises isolating a cell that produces an antibody that exhibits the binding properties of (i) and (ii) and optionally (iii).

92. The method according to any one of claims 88-91 that comprises a step, prior to the assaying step, of immunizing a non-human mammal with the one or more monomeric TTR peptides, and the assaying comprises screening antibody molecules produced by the mammal.

93. The method according to any one of claims 88-92, further comprising:
determining amino acid sequence of at least the complementarity-determining regions (CDR) of the antibody;
generating humanized antibody sequences that includes said amino acid sequences of said CDRs; and
synthesizing nucleic acid that comprises nucleotide sequences that encode the humanized antibody.

94. The method according to claim 93, further comprising transfecting a cell with a nucleic acid that encodes the humanized antibody sequence, or with a nucleic acid that encodes an antigen-binding fragment of the humanized antibody sequence.

95. The method of claim 94, further comprising culturing the cell under conditions to express the antibody or the antigen-binding fragment.

96. The method according to claim 88 or 89, further comprising synthesizing copies of the molecule identified as having the binding properties of (i) and (ii) and, optionally (iii).

97. An isolated molecule produced by the process of claim 95 or 96.

98. A pharmaceutical composition comprising the isolated molecule of claim 97 and a pharmaceutically acceptable carrier.


SEQUENCE LISTING (153 sequences)

SEQ ID NO: 1 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 2 - protein, 8 aa, synthetic construct
  REGION 1..8: note = Synthetic
GYTFTSYW

SEQ ID NO: 3 - protein, 8 aa, synthetic construct
  REGION 1..8: note = Synthetic
GFTFSNYW

SEQ ID NO: 4 - protein, 8 aa, synthetic construct
  REGION 1..8: note = Synthetic
GYTFTDYY

SEQ ID NO: 5 - protein, 8 aa, synthetic construct
  REGION 1..8: note = Synthetic
GYTFTTYG

SEQ ID NO: 6 - protein, 8 aa, synthetic construct
  REGION 1..8: note = Synthetic
GFNIKDYY

SEQ ID NO: 7 - protein, 8 aa, synthetic construct
  REGION 1..8: note = Synthetic
GYTFTGYW

SEQ ID NO: 8 - protein, 8 aa, synthetic construct
  REGION 1..8: note = Synthetic
GFNIKDYY

SEQ ID NO: 9 - protein, 8 aa, synthetic construct
  REGION 1..8: note = Synthetic
IDPSDSET

SEQ ID NO: 10 - protein, 10 aa, synthetic construct
  REGION 1..10: note = Synthetic
IRLKSDNYAT

SEQ ID NO: 11 - protein, 8 aa, synthetic construct
  REGION 1..8: note = Synthetic
INPYNGGT

SEQ ID NO: 12 - protein, 8 aa, synthetic construct
  REGION 1..8: note = Synthetic
INTYSGVP

SEQ ID NO: 13 - protein, 8 aa, synthetic construct
  REGION 1..8: note = Synthetic
IDPEDGET

SEQ ID NO: 14 - protein, 8 aa, synthetic construct
  REGION 1..8: note = Synthetic
ILPGSGST

SEQ ID NO: 15 - protein, 8 aa, synthetic construct
  REGION 1..8: note = Synthetic
IDPEDGET

SEQ ID NO: 16 - protein, 11 aa, synthetic construct
  REGION 1..11: note = Synthetic
ARGRNYWYFDV

SEQ ID NO: 17 - protein, 6 aa, synthetic construct
  REGION 1..6: note = Synthetic
TGVFTY

SEQ ID NO: 18 - protein, 13 aa, synthetic construct
  REGION 1..13: note = Synthetic
ARDLTGTGGAMDY

SEQ ID NO: 19 - protein, 12 aa, synthetic construct
  REGION 1..12: note = Synthetic
AFYYDYQEWFAY

SEQ ID NO: 20 - protein, 11 aa, synthetic construct
  REGION 1..11: note = Synthetic
ALLWLRRGRGY

SEQ ID NO: 21 - protein, 14 aa, synthetic construct
  REGION 1..14: note = Synthetic
ARLRGSSYPWYFDY

SEQ ID NO: 22 - protein, 9 aa, synthetic construct
  REGION 1..9: note = Synthetic
ARGKTPFAY

SEQ ID NO: 23 - protein, 10 aa, synthetic construct
  REGION 1..10: note = Synthetic
ESVDNYGISF

SEQ ID NO: 24 - protein, 10 aa, synthetic construct
  REGION 1..10: note = Synthetic
ESVDSYGNSF

SEQ ID NO: 25 - protein, 5 aa, synthetic construct
  REGION 1..5: note = Synthetic
SSVSY

SEQ ID NO: 26 - protein, 6 aa, synthetic construct
  REGION 1..6: note = Synthetic
ENIYSN

SEQ ID NO: 27 - protein, 6 aa, synthetic construct
  REGION 1..6: note = Synthetic
QEISGY

SEQ ID NO: 28 - protein, 8 aa, synthetic construct
  REGION 1..8: note = Synthetic
GYTFTGYW

SEQ ID NO: 29 - protein, 6 aa, synthetic construct
  REGION 1..6: note = Synthetic
ENIYSN

SEQ ID NO: 30-36 - skipped (7 entries)

SEQ ID NO: 37 - protein, 9 aa, synthetic construct
  REGION 1..9: note = Synthetic
QQSKEVPWT

SEQ ID NO: 38 - protein, 10 aa, synthetic construct
  REGION 1..10: note = Synthetic
QQNNEDPMYT

SEQ ID NO: 39 - protein, 9 aa, synthetic construct
  REGION 1..9: note = Synthetic
QQWSSNPLT

SEQ ID NO: 40 - protein, 8 aa, synthetic construct
  REGION 1..8: note = Synthetic
QHFWGTPR

SEQ ID NO: 41 - protein, 9 aa, synthetic construct
  REGION 1..9: note = Synthetic
LQYASYPPT

SEQ ID NO: 42 - protein, 9 aa, synthetic construct
  REGION 1..9: note = Synthetic
QHFWGTPYT

SEQ ID NO: 43 - protein, 9 aa, synthetic construct
  REGION 1..9: note = Synthetic
LQYDSYPFT

SEQ ID NO: 44 - skipped

SEQ ID NO: 45 - protein, 118 aa, synthetic construct
  REGION 1..118: note = Synthetic
QVQLQQPGAELVRPGSSVKLSCKASGYTFTSYWMHWVKQRPIQGLEWIGNIDPSDSETHYNQKFKDKATLTVDKSSSTAYMQLSSLTSEDSAVYYCARGRNYWYFDVWGTGTTVTVSS

SEQ ID NO: 46 - protein, 118 aa, synthetic construct
  REGION 1..118: note = Synthetic
QVQLVQSGAEVKKPGASVKVSCKASGYTFTSYWMHWVRQAPGQGLEWMGNIDPSDSETHYAQKFQGRVTMTRDTSTSTVYMELSSLRSEDTAVYYCARGRNYWYFDVWGTGTMVTVSS

SEQ ID NO: 47 - protein, 118 aa, synthetic construct
  REGION 1..118: note = Synthetic
QVQLVQSGAEVKKPGASVKVSCKASGYTFTSYWMHWVRQAPGQGLEWMGNIDPSDSETHYAQKFQGRVTMTVDTSTSTVYMELSSLRSEDTAVYYCARGRNYWYFDVWGTGTMVTVSS

SEQ ID NO: 48 - protein, 118 aa, synthetic construct
  REGION 1..118: note = Synthetic
QVQLVQSGAEVKKPGASVKVSCKASGYTFTSYWMHWVRQAPGQGLEWMGNIDPSDSETHYAQKFQGRVTMTVDKSTSTVYMELSSLRSEDTAVYYCARGRNYWYFDVWGTGTMVTVSS

SEQ ID NO: 49 - protein, 118 aa, synthetic construct
  REGION 1..118: note = Synthetic
QVQLVQSGAEVKKPGASVKVSCKASGYTFTSYWMHWVRQAPGQGLEWIGNIDPSDSETHYAQKFQGRATLTVDKSTSTVYMELSSLRSEDTAVYYCARGRNYWYFDVWGTGTMVTVSS

SEQ ID NO: 50 - protein, 111 aa, synthetic construct
  REGION 1..111: note = Synthetic
DIVLTQSPASLAVSLGQRATISCRASESVDNYGISFMNWFQQKPGQPPKLLIYAASNQGSGVPARFSGSGSGTDFSLNIHPMEEDDTAMYFCQQSKEVPWTFGGGTKLEIK

SEQ ID NO: 51 - protein, 111 aa, synthetic construct
  REGION 1..111: note = Synthetic
DIQMTQSPSSLSASVGDRVTITCRASESVDNYGISFMNWYQQKPGKAPKLLIYAASNQGSGVPSRFSGSGSGTDFTLTISSLQPEDFATYYCQQSKEVPWTFGGGTKLEIK

SEQ ID NO: 52 - protein, 111 aa, synthetic construct
  REGION 1..111: note = Synthetic
DIQMTQSPSSLSASVGDRVTITCRASESVDNYGISFMNWFQQKPGKAPKLLIYAASNQGSGVPSRFSGSGSGTDFTLTISSLQPEDFATYFCQQSKEVPWTFGGGTKLEIK

SEQ ID NO: 53 - protein, 111 aa, synthetic construct
  REGION 1..111: note = Synthetic
DIQMTQSPSSLSASVGDRVTITCRASESVDNYGISFMNWFQQKPGKPPKLLIYAASNQGSGVPSRFSGSGSGTDFTLTISSLQPEDFATYYCQQSKEVPWTFGGGTKLEIK

SEQ ID NO: 54 - protein, 115 aa, synthetic construct
  REGION 1..115: note = Synthetic
EVKLEQSGGGLVQPGGSMKLSCVASGFTFSNYWMNWVRQSPEKGLEWVAQIRLKSDNYATHYAESVKGRFTISRDDSKSSVYLQMNNLRAEDTGIYYCTGVFTYWGQGTLVTVSA

SEQ ID NO: 55 - protein, 115 aa, synthetic construct
  REGION 1..115: note = Synthetic
EVQLVESGGGLVKPGGSLRLSCAASGFTFSNYWMNWVRQAPGKGLEWVGQIRLKSDNYATHYAAPVKGRFTISRDDSKNTLYLQMNSLKTEDTAVYYCTTVFTYWGQGTLVTVSS

SEQ ID NO: 56 - protein, 115 aa, synthetic construct
  REGION 1..115: note = Synthetic
EVQLVESGGGLVKPGGSLRLSCAASGFTFSNYWMNWVRQAPGKGLEWVGQIRLKSDNYATHYAAPVKGRFTISRDDSKNTLYLQMNSLKTEDTAVYYCTGVFTYWGQGTLVTVSS

SEQ ID NO: 57 - protein, 115 aa, synthetic construct
  REGION 1..115: note = Synthetic
EVQLVESGGGLVKPGGSLRLSCAASGFTFSNYWMNWVRQAPGKGLEWVAQIRLKSDNYATHYAAPVKGRFTISRDDSKNTVYLQMNSLKTEDTAVYYCTGVFTYWGQGTLVTVSS

SEQ ID NO: 58 - protein, 115 aa, synthetic construct
  REGION 1..115: note = Synthetic
EVQLLESGGGLVQPGGSLRLSCAASGFTFSNYWMNWVRQAPGKGLEWVSQIRLKSDNYATHYADSVKGRFTISRDNSKNTLYLQMNSLRAEDTAVYYCAKVFTYWGQGTLVTVSS

SEQ ID NO: 59 - protein, 115 aa, synthetic construct
  REGION 1..115: note = Synthetic
EVQLLESGGGLVQPGGSLRLSCAASGFTFSNYWMNWVRQAPGKGLEWVSQIRLKSDNYATHYADSVKGRFTISRDNSKNTLYLQMNSLRAEDTAVYYCTGVFTYWGQGTLVTVSS

SEQ ID NO: 60 - protein, 115 aa, synthetic construct
  REGION 1..115: note = Synthetic
EVQLLESGGGLVQPGGSLRLSCAASGFTFSNYWMNWVRQAPGKGLEWVAQIRLKSDNYATHYADSVKGRFTISRDDSKNTVYLQMNSLRAEDTAVYYCTGVFTYWGQGTLVTVSS

SEQ ID NO: 61 - protein, 112 aa, synthetic construct
  REGION 1..112: note = Synthetic
NIVLTQSPASLAVSLGQRATISCRTSESVDSYGNSFMHWYQQKPGQPPKLLIYLASNLESGVPARFSGSGSRTDFTLTIDPVEADDAATYYCQQNNEDPMYTFGGGTKLEIK

SEQ ID NO: 62 - protein, 112 aa, synthetic construct
  REGION 1..112: note = Synthetic
DIVMTQSPDSLAVSLGERATINCRTSESVDSYGNSFMHWYQQKPGQPPKLLIYLASNLESGVPDRFSGSGSGTDFTLTISSLQAEDVAVYYCQQNNEDPMYTFGGGTKVEIK

SEQ ID NO: 63 - protein, 112 aa, Homo sapiens
DIVMTQSPDSLAVSLGERATINCRTSESVDSYGNSFMHWYQQKPGQPPKLLIYLASNLESGVPDRFSGSGSRTDFTLTISSLQAEDVAVYYCQQNNEDPMYTFGGGTKVEIK

SEQ ID NO: 64 - protein, 112 aa, synthetic construct
  REGION 1..112: note = Synthetic
DIVLTQSPDSLAVSLGERATINCRTSESVDSYGNSFMHWYQQKPGQPPKLLIYLASNLESGVPDRFSGSGSRTDFTLTISSLQAEDVAVYYCQQNNEDPMYTFGGGTKVEIK

SEQ ID NO: 65 - protein, 12 aa, synthetic construct
  REGION 1..12: note = Synthetic
GPTGTGESKCPL

SEQ ID NO: 66 - protein, 13 aa, synthetic construct
  REGION 1..13: note = Synthetic
MVKVLDAVRGSPA

SEQ ID NO: 67 - protein, 12 aa, synthetic construct
  REGION 1..12: note = Synthetic
PFHEHAEVVFTA

SEQ ID NO: 68 - protein, 9 aa, synthetic construct
  REGION 1..9: note = Synthetic
EHAEVVFTA

SEQ ID NO: 69 - protein, 8 aa, synthetic construct
  REGION 1..8: note = Synthetic
YTIAALLS

SEQ ID NO: 70 - protein, 6 aa, synthetic construct
  REGION 1..6: note = Synthetic
LSPYSY

SEQ ID NO: 71 - protein, 8 aa, synthetic construct
  REGION 1..8: note = Synthetic
SYSTTAVV

SEQ ID NO: 72 - protein, 18 aa, synthetic construct
  REGION 1..18: note = Synthetic
YTIAALLSPYSYSTTAVV

SEQ ID NO: 73 - protein, 11 aa, synthetic construct
  REGION 1..11: note = Synthetic
LGISPMHEHAE

SEQ ID NO: 74 - protein, 13 aa, synthetic construct
  REGION 1..13: note = Synthetic
RRYTIAAMLSPYS

SEQ ID NO: 75 - skipped

SEQ ID NO: 76 - protein, 148 aa, synthetic construct
  REGION 1..148: note = Synthetic
MGSSHHHHHHSSGLVPAGSHMGPTGTGESKCPLMVKVLDAVRGSPAINVAVHVFRKAADDTWEPFASGKTSESGELHGLTTEEEFVEGIYKVEIDTKSYWKALGISPMHEHAEVVFTANDSGPRRYTIAAMLSPYSYSTTAVVTNPKE

SEQ ID NO: 77 - protein, 15 aa, synthetic construct
  REGION 1..15: note = Synthetic
GLNDIFEAQKIEWHE

SEQ ID NO: 78 - protein, 162 aa, synthetic construct
  REGION 1..162: note = Synthetic
MHHHHHHSGMSGLNDIFEAQKIEWHEGENLYFQGMGPTGTGESKCPLMVKVLDAVRGSPAINVAVHVFRKAADDTWEPFASGKTSESGELHGLTTEEEFVEGIYKVEIDTKSYWKALGISPMHEHAEVVFTANDSGPRRYTIAAMLSPYSYSTTAVVTNPKE

SEQ ID NO: 79 - skipped

SEQ ID NO: 80 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPMHEHAEVVFTANDSGPRRYTIAAMLSPYSYSTTAVVTNPKE

SEQ ID NO: 81 - protein, 445 aa, synthetic construct
  REGION 1..445: note = Synthetic
QVQLQQPGAELVRPGSSVKLSCKASGYTFTSYWMHWVKQRPIQGLEWIGNIDPSDSETHYNQKFKDKATLTVDKSSSTAYMQLSSLTSEDSAVYYCARGRNYWYFDVWGTGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 442 aa, synthetic construct
  REGION 1..442: note = Synthetic
EVKLEQSGGGLVQPGGSMKLSCVASGFTFSNYWMNWVRQSPEKGLEWVAQIRLKSDNYATHYAESVKGRFTISRDDSKSSVYLQMNNLRAEDTGIYYCTGVFTYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 83 - protein, 447 aa, synthetic construct
  REGION 1..447: note = Synthetic
EVQLQQSGPVLVKPGASVKMSCKASGYTFTDYYMNWVKQSHGKSLEWIGVINPYNGGTSYNQKFKGKATLTVDKSSSTAYMELNSLTSEDSAVYYCARDLTGTGGAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 84 - protein, 445 aa, synthetic construct
  REGION 1..445: note = Synthetic
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCALLWLRRGRGYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 85 - protein, 448 aa, synthetic construct
  REGION 1..448: note = Synthetic
QVQLQQSGAELMKPGASVKLSCKATGYTFTGYWIEWVKQRPGHGLEWIGEILPGSGSTNYNEKFKGKATFTADTSSNTAYMQLSSLTTEDSAIYYCARLRGSSYPWYFDYWGQGTTLTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 86 - protein, 443 aa, synthetic construct
  REGION 1..443: note = Synthetic
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCARGKTPFAYWGQGTLVTVSAASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 87 - protein, 118 aa, Mus musculus; identical to SEQ ID NO: 45

SEQ ID NO: 88 - protein, 118 aa, Homo sapiens; identical to SEQ ID NO: 46

SEQ ID NO: 89 - protein, 118 aa, Homo sapiens; identical to SEQ ID NO: 47

SEQ ID NO: 90 - protein, 118 aa, Homo sapiens; identical to SEQ ID NO: 48

SEQ ID NO: 91 - protein, 118 aa, Homo sapiens; identical to SEQ ID NO: 49

SEQ ID NO: 92 - protein, 111 aa, Mus musculus; identical to SEQ ID NO: 50

SEQ ID NO: 93 - protein, 111 aa, Homo sapiens; identical to SEQ ID NO: 51

SEQ ID NO: 94 - protein, 111 aa, Homo sapiens; identical to SEQ ID NO: 52

SEQ ID NO: 95 - protein, 111 aa, Homo sapiens; identical to SEQ ID NO: 53

SEQ ID NO: 96 - protein, 115 aa, Mus musculus; identical to SEQ ID NO: 54

SEQ ID NO: 97 - protein, 115 aa, Homo sapiens; identical to SEQ ID NO: 55

SEQ ID NO: 98 - protein, 115 aa, Homo sapiens; identical to SEQ ID NO: 56

SEQ ID NO: 99 - protein, 115 aa, Homo sapiens; identical to SEQ ID NO: 57

SEQ ID NO: 100 - protein, 115 aa, Homo sapiens; identical to SEQ ID NO: 58

SEQ ID NO: 101 - protein, 115 aa, Homo sapiens; identical to SEQ ID NO: 59

SEQ ID NO: 102 - protein, 115 aa, Homo sapiens; identical to SEQ ID NO: 60

SEQ ID NO: 103 - protein, 112 aa, Mus musculus; identical to SEQ ID NO: 61

SEQ ID NO: 104 - protein, 112 aa, Homo sapiens; identical to SEQ ID NO: 62

SEQ ID NO: 105 - protein, 112 aa, Homo sapiens; identical to SEQ ID NO: 63

SEQ ID NO: 106 - protein, 112 aa, Homo sapiens; identical to SEQ ID NO: 64

SEQ ID NO: 107 - protein, 120 aa, Mus musculus
EVQLQQSGPVLVKPGASVKMSCKASGYTFTDYYMNWVKQSHGKSLEWIGVINPYNGGTSYNQKFKGKATLTVDKSSSTAYMELNSLTSEDSAVYYCARDLTGTGGAMDYWGQGTSVTVSS

SEQ ID NO: 108 - protein, 120 aa, Homo sapiens
QVQLVQSGAEVKKPGASVKVSCKASGYTFTDYYMNWVRQAPGQGLEWMGVINPYNGGTSYAQKFQGRVTMTRDTSTSTVYMELSSLRSEDTAVYYCARDLTGTGGAMDYWGQGTLVTVSS

SEQ ID NO: 109 - protein, 120 aa, Homo sapiens
QVQLVQSGAEVKKPGASVKVSCKASGYTFTDYYMHWVRQAPGQGLEWMGVINPYNGGTSYAQKFQGRVTMTVDKSTSTVYMELSSLRSEDTAVYYCARDLTGTGGAMDYWGQGTLVTVSS

SEQ ID NO: 110 - protein, 120 aa, Homo sapiens
QVQLVQSGAEVKKPGASVKVSCKASGYTFTDYYMHWVRQAPGQGLEWIGVINPYNGGTSYAQKFQGRATLTVDKSTSTAYMELSSLRSEDTAVYYCARDLTGTGGAMDYWGQGTLVTVSS

SEQ ID NO: 111 - protein, 120 aa, Homo sapiens
QVQLVQSGAEVKKPGASVKVSCKASGYTFTDYYMHWVRQAPGQSLEWIGVINPYNGGTSYAQKFQGRATLTVDKSTSTAYMELSSLRSEDTAVYYCARDLTGTGGAMDYWGQGTLVTVSS

SEQ ID NO: 112 - protein, 106 aa, Mus musculus
QIVLTQSPAILSASPGEKVTMTCRASSSVSYMHWYQQKPGSSPKPWIYATSNLASGVPARFSGSGSGTSYSLTISRVEAEDAATYYCQQWSSNPLTFGAGTKLELK

SEQ ID NO: 113 - protein, 106 aa, Homo sapiens
EIVLTQSPGTLSLSPGERATLSCRASSSVSYMHWYQQKPGQAPRLLIYATSNLASGIPDRFSGSGSGTDFTLTISSLEPEDFAVYYCQQWSSNPLTFGGGTKVEIK

SEQ ID NO: 114 - protein, 106 aa, Homo sapiens
EIVLTQSPGTLSLSPGERATLSCRASSSVSYMHWYQQKPGQAPRPWIYATSNLASGIPDRFSGSGSGTDFTLTISSLEPEDFAVYYCQQWSSNPLTFGGGTKVEIK

SEQ ID NO: 115 - protein, 106 aa, Homo sapiens
EIVLTQSPGTLSLSPGERATLSCRASSSVSYMHWYQQKPGQAPRPWIYATSNLASGIPDRFSGSGSGTDYTLTISSLEPEDFAVYYCQQWSSNPLTFGGGTKVEIK

SEQ ID NO: 116 - protein, 106 aa, Homo sapiens
EIVLTQSPGTLSLSPGERATLSCRASSSVSYMHWYQQKPGQSPRPWIYATSNLASGIPDRFSGSGSGTDYTLTISSLEPEDFAVYYCQQWSSNPLTFGGGTKVEIK

SEQ ID NO: 117 - protein, 118 aa, Mus musculus
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCALLWLRRGRGYWGQGTTLTVSS

SEQ ID NO: 118 - protein, 118 aa, Homo sapiens
QVQLVQSGAEVKKPGSSVKVSCKASGFNIKDYYMHWVRQAPGQGLEWMGRIDPEDGETKYAQKFQGRVTITADESTSTAYMELSSLRSEDTAVYYCARLWLRRGRGYWGQGTLVTVSS

SEQ ID NO: 119 - protein, 118 aa, Homo sapiens
QVQLVQSGAEVKKPGSSVKVSCKASGFNIKDYYMHWVRQAPGQGLEWMGRIDPEDGETKYAQKFQGRVTITADTSTSTAYMELSSLRSEDTAVYYCALLWLRRGRGYWGQGTLVTVSS

SEQ ID NO: 120 - protein, 118 aa, Homo sapiens
QVQLVQSGAEVKKPGSSVKVSCKASGFNIKDYYMHWVRQAPGQGLEWIGRIDPEDGETKYAQKFQGRATITADTSTSTAYMELSSLRSEDTAVYYCALLWLRRGRGYWGQGTLVTVSS

SEQ ID NO: 121 - protein, 107 aa, Mus musculus
DIQMTQSPSSLSASLGERVSLTCRASQEISGYLSWLQQKPDGTIKRLIYAASTLDSGVPKRFSGSRSGSDYSLTISSLESEDFADYYCLQYASYPPTFGAGTKLELK

SEQ ID NO: 122 - protein, 107 aa, Homo sapiens
DIQMTQSPSSLSASVGDRVTITCRASQEISGYLSWYQQKPGKAPKLLIYAASTLDSGVPSRFSGSGSGTDFTLTISSLQPEDFATYYCLQYASYPPTFGGGTKVEIK

SEQ ID NO: 123 - protein, 107 aa, Homo sapiens
DIQMTQSPSSLSASVGDRVTITCRASQEISGYLSWLQQKPGKAPKRLIYAASTLDSGVPSRFSGSRSGTDFTLTISSLQPEDFATYYCLQYASYPPTFGGGTKVEIK

SEQ ID NO: 124 - protein, 107 aa, Homo sapiens
DIQMTQSPSSLSASVGDRVTITCRASQEISGYLSWLQQKPGKTIKRLIYAASTLDSGVPSRFSGSRSGTDFTLTISSLQPEDFATYYCLQYASYPPTFGGGTKVEIK

SEQ ID NO: 125 - protein, 107 aa, Homo sapiens
DIQMTQSPSSLSASVGDRVTITCRASQEISGYLSWLQQKPGKTIKRLIYAASTLDSGVPSRFSGSRSGTDYTLTISSLQPEDFATYYCLQYASYPPTFGGGTKVEIK

SEQ ID NO: 126 - protein, 121 aa, Mus musculus
QVQLQQSGAELMKPGASVKLSCKATGYTFTGYWIEWVKQRPGHGLEWIGEILPGSGSTNYNEKFKGKATFTADTSSNTAYMQLSSLTTEDSAIYYCARLRGSSYPWYFDYWGQGTTLTVSS

SEQ ID NO: 127 - protein, 121 aa, Homo sapiens
QVQLVQSGAEVKKPGSSVKVSCKASGYTFTGYWIEWVRQAPGQGLEWMGEILPGSGSTNYAQKFQGRVTITADESTSTAYMELSSLRSEDTAVYYCARLRGSSYPWYFDYWGQGTLVTVSS

SEQ ID NO: 128 - protein, 121 aa, Homo sapiens
QVQLVQSGAEVKKPGSSVKVSCKASGYTFTGYWIEWVRQAPGQGLEWMGEILPGSGSTNYAQKFQGRVTFTADTSTSTAYMELSSLRSEDTAVYYCARLRGSSYPWYFDYWGQGTLVTVSS

SEQ ID NO: 129 - protein, 121 aa, Homo sapiens
QVQLVQSGAEVKKPGSSVKVSCKASGYTFTGYWIEWVRQAPGQGLEWIGEILPGSGSTNYAQKFQGRATFTADTSTSTAYMELSSLRSEDTAVYYCARLRGSSYPWYFDYWGQGTLVTVSS

SEQ ID NO: 130 - protein, 107 aa, Mus musculus
DIQMTQSPASLSVSVGETVTITCRASENIYSNLAWYQQKQGKSPQLLVYAATNLADGVPSRFSGSGSGTQYSLKINSLQSEDFGSYYCQHFWGTPYTFGGGTKLEIK

SEQ ID NO: 131 - protein, 107 aa, Homo sapiens
DIQMTQSPSSLSASVGDRVTITCRASENIYSNLAWYQQKPGKAPKLLIYAATNLADGVPSRFSGSGSGTDFTLTISSLQPEDFATYYCQHFWGTPYTFGGGTKVEIK

SEQ ID NO: 132 - protein, 107 aa, Homo sapiens
DIQMTQSPSSLSASVGDRVTITCRASENIYSNLAWYQQKPGKAPKLLVYAATNLADGVPSRFSGSGSGTDYTLTISSLQPEDFATYYCQHFWGTPYTFGGGTKVEIK

SEQ ID NO: 133 - protein, 107 aa, Homo sapiens
DIQMTQSPSSLSASVGDRVTITCRASENIYSNLAWYQQKPGKSPKLLVYAATNLADGVPSRFSGSGSGTDYTLTISSLQPEDFATYYCQHFWGTPYTFGGGTKVEIK

SEQ ID NO: 134 - protein, 116 aa, Mus musculus
EVQLQQSGAELVKPGASVKLSCTASGFNIKDYYMHWVKQRTEQGLEWIGRIDPEDGETKYAPKFQGKATITADTSSNTAYLQLSSLTSEDTAVYYCARGKTPFAYWGQGTLVTVSA

SEQ ID NO: 135 - protein, 116 aa, Homo sapiens
QVQLVQSGAEVKKPGSSVKVSCKASGFNIKDYYMHWVRQAPGQGLEWMGRIDPEDGETKYAQKFQGRVTITADESTSTAYMELSSLRSEDTAVYYCARGKTPFAYWGQGTLVTVSS

SEQ ID NO: 136 - protein, 116 aa, Homo sapiens
QVQLVQSGAEVKKPGSSVKVSCKASGFNIKDYYMHWVRQAPGQGLEWMGRIDPEDGETKYAQKFQGRVTITADTSTNTAYMELSSLRSEDTAVYYCARGKTPFAYWGQGTLVTVSS

SEQ ID NO: 137 - protein, 116 aa, Homo sapiens
QVQLVQSGAEVKKPGSSVKVSCKASGFNIKDYYMHWVRQAPGQGLEWIGRIDPEDGETKYAQKFQGKATITADTSSNTAYMELSSLRSEDTAVYYCARGKTPFAYWGQGTLVTVSS

SEQ ID NO: 138 - protein, 107 aa, Mus musculus
DIQMTQSPSSLSASLGERVSLTCRASQEISGYLSWLQQKPDGTIKRLIYAASTLDFGVPKRFSGSRFGSDYSLTISSLESEDFADYYCLQYDSYPFTFGSGTKLEIK

SEQ ID NO: 139 - protein, 107 aa, synthetic construct
  REGION 1..107: note = Synthetic
DIQMTQSPSSLSASVGDRVTITCRASQEISGYLSWYQQKPGKAPKLLIYAASTLDFGVPSRFSGSGSGTDFTLTISSLQPEDFATYYCLQYDSYPFTFGGGTKVEIK

SEQ ID NO: 140 - protein, 107 aa, Homo sapiens
DIQMTQSPSSLSASVGDRVTITCRASQEISGYLSWLQQKPGKAPKRLIYAASTLDFGVPSRFSGSRSGTDFTLTISSLQPEDFATYYCLQYDSYPFTFGGGTKVEIK

SEQ ID NO: 141 - protein, 107 aa, Homo sapiens
DIQMTQSPSSLSASVGDRVTITCRASQEISGYLSWLQQKPGKAPKRLIYAASTLDFGVPSRFSGSRSGSDFTLTISSLQPEDFATYYCLQYDSYPFTFGGGTKVEIK

SEQ ID NO: 142 - protein, 107 aa, Homo sapiens
DIQMTQSPSSLSASVGDRVTITCRASQEISGYLSWLQQKPGKTIKRLIYAASTLDFGVPSRFSGSRSGSDFTLTISSLQPEDFATYYCLQYDSYPFTFGGGTKVEIK

SEQ ID NO: 143 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPMHEHAEVVFTANDSGPRRYTIAALLSPYSYSTTAVVTNPKE

SEQ ID NO: 144 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAAMLSPYSYSTTAVVTNPKE

SEQ ID NO: 145 - protein, 127 aa, synthetic construct
  REGION 1..127: note = Synthetic
GPTGTGESKCPLMVKVLDAVRGSPAINVAMHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 146 - protein, 127 aa, synthetic construct
  REGION 1..127: note = Synthetic
GPTGTGESKCPLMVKVLDAVRGSPAINVAVHVFRKAAADTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 147 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVITNPKE

SEQ ID NO: 148 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLIPYSYSTTAVVTNPKE

SEQ ID NO: 149 - protein, 127 aa, synthetic construct
  REGION 1..127: note = Synthetic
GPTGTGESKCPLMVKVLDAVRGSPAINVAMHVFRKAADDTWEPFASGKTSESGELHGLTTEEEFVEGIYKVEIDTKSYWKALGISPMHEHAEVVFTANDSGPRRYTIAAMLSPYSYSTTAVVTNPKE

SEQ ID NO: 150 - protein, 127 aa, synthetic construct
  REGION 1..127: note = Synthetic
GPTGTGESKCPLMVKVLDAVRGSPAINVAVHVFRKAAADTWEPFASGKTSESGELHGLTTEEEFVEGIYKVEIDTKSYWKALGISPMHEHAEVVFTANDSGPRRYTIAAMLSPYSYSTTAVVTNPKE

SEQ ID NO: 151 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPMHEHAEVVFTANDSGPRRYTIAAMLSPYSYSTTAVITNPKE

SEQ ID NO: 152 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPMHEHAEVVFTANDSGPRRYTIAAMLIPYSYSTTAVVTNPKE

SEQ ID NO: 153 - protein, 127 aa, synthetic construct
  REGION 1..127: note = Synthetic
GPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTMAVVTNPKE